(19) United States
(12) Patent Application Publication
(10) Pub. No.: US 2021/0147381 A1
(43) Pub. Date: May 20, 2021

(54) 1,2,3,4-SUBSTITUTED QUINOLINE COMPOUNDS AS SIP MODULATORS

(71) Applicant: AbbVie Deutschland GmbH & Co. KG, Wiesbaden (DE)

(72) Inventors: Wilhelm AMBERG, Ludwigshafen (DE); Hervé GENESTE, Ludwigshafen (DE); Wilfried HORNBERGER, Ludwigshafen (DE); Udo LANGE, Ludwigshafen (DE); Mario MEZLER, Ludwigshafen (DE); Michael OCHSE, Ludwigshafen (DE); Frank OELLIEN, Ludwigshafen (DE); Sean C. TURNER, Ludwigshafen (DE); Jeroen VAN BERGEIJK, Ludwigshafen (DE); Elizabeth VAN DER KAM, Ludwigshafen (DE)

(21) Appl. No.: 16/630,813

(22) PCT Filed: Jul. 16, 2018
(86) PCT No.: PCT/EP2018/069196

§ 371 Date: Jan. 13, 2020

(30) Foreign Application Priority Data
Jul. 17, 2017 (US) 62533316

(51) Int. Cl. C07D 401/06; C07D 217/02; C07D 409/06

(52) U.S. Cl. CPC C07D 401/06; C07D 409/06; C07D 217/02

(57) ABSTRACT

The invention relates to 1,2,3,4-substituted quinoline compounds as S1P modulators, pharmaceutical compositions comprising such compounds, and uses thereof in the treatment, alleviation or prevention of diseases or disorders mediated by an S1P receptor.

The invention relates to 1,2,3,4-substituted hydroquinoline compounds with affinity to S1P receptors, pharmaceutical compositions comprising such compounds, the use of such compounds in the treatment, alleviation or prevention of diseases and conditions in which an S1P receptor is involved or in which modulation of the endogenous S1P signaling system via an S1P receptor is involved and the preparation of a medicament for treating, alleviating or preventing such diseases and conditions.

### BACKGROUND OF THE INVENTION

Sphingosine-1-phosphate (S1P) is part of the sphingolipid class of molecules. S1P is a bioactive sphingolipid that mediates a wide variety of cellular responses, such as proliferation, autophagy, blockade of apoptosis, cell differentiation, blockade of cell senescence, cytoskeletal organization and migration, adherence- and tight junction assembly, and morphogenesis. Moreover, S1P is a modulator of APP processing via BACE1 regulation as well as lipid raft formation and can interact with ABC transporters thereby modulating cellular in- and efflux. S1P can bind with members of the endothelial cell differentiation gene family (EDG receptors) of plasma membrane-localized G protein-coupled receptors. To date, five members of this family have been identified as S1P receptors in different cell types, S1P1 (EDG-1), S1P2 (EDG-5), S1P3 (EDG-3), S1P4 (EDG-6) and S1P5 (EDG-8). S1P can produce cytoskeletal re-arrangements in many cell types to regulate immune cell trafficking, vascular homeostasis and cell communication in the central nervous system (CNS) and in peripheral organ systems. The above mentioned actions of S1P are mediated by interaction with its receptors. Therefore, S1P receptors are therapeutic targets for the treatment of, for example, neoplastic diseases, diseases of the central and peripheral nervous system, autoimmune disorders and tissue rejection in transplantation.

It is known that SP is secreted by vascular endothelium and is present in blood at concentrations of 200-900 nanomolar and is bound by albumin and other plasma proteins. This provides both a stable reservoir in extracellular fluids and efficient delivery to high-affinity cell-surface receptors. S1P binds with low nanomolar affinity to the five receptors S1P1-5. In addition, platelets also contain S1P and may be locally released to cause e.g. vasoconstriction. The receptor subtypes S1P1, S1P2 and S1P3 are widely expressed and represent dominant receptors in the cardiovascular system. Further, S1P1 is also a receptor on lymphocytes. S1P4 receptors are almost exclusively in the haematopoietic and lymphoid system. S1P5 is primarily (though not exclusively) expressed in central nervous system (CNS; brain and spinal cord). Other tissues with S1P5 expression are skin and spleen. Moreover, S1P5 is expressed on NK cells. Early study showed that the CNS expression in mice appeared restricted to oligodendrocytes, while in men and rats expression was more diverse. Recent evidence has shown a broader distribution in all species: S1P5 expression is shown at the level of astrocytes, endothelial cells, glial cells, oligodendrocytes and to a lesser extent neurons.

The present invention relates to modulators of the S1P5 receptor, in particular agonists, and preferably to agonists with selectivity over S1P1, S1P3 and/or S1P4 receptors, in view of unwanted cardiovascular and/or peripheral immune-modulatory effects. It has now been found that S1P5 agonists can be used in the treatment of cognitive disorders, in particular age-related cognitive decline. Moreover, evidence has shown an impact on amyloid B (protein) processing, ABC transporter expression, blood-brain-barrier integrity, neuro-inflammatory processes, and (sphingo)lipid content in the CNS.

The latter is of high relevance as an altered sphingolipid metabolism is strongly implicated in several neurodegenerative and cognitive diseases. A comparison of CNS gene expression profiles of normal and Alzheimer's Disease (AD) patients indicated that genes responsible for S1P degradation were strongly upregulated, including the phosphatidic acid phosphatase PPAP2A and S1P lyase genes, while genes for ceramide production (apoptotic sphingolipid) were upregulated (Katsel et al, 2007, Neurochem Res, 32, 845-856). These gene expression data are predictive of actual changes in enzyme and lipid levels in the brain and cerebrospinal fluid (CSF): compared to normal subjects, AD brain are characterized by higher levels of ceramide and cholesterol as well as decreased levels of S1P. These changes also correlate with disease severity of the patients and are related to levels of Amyloid 6 and Tau, two hallmarks of Alzheimer's Disease (Cutler et al, 2004, PNAS, 101, 2070-2075; He et al, 2010, Neurobiol. Aging, 31, 398-408; Koal et al, 2015, J. Alz Disease, 44, 1193-1201). The same changes have been reported in brain tissues (and CSF) from patients suffering HIV dementia, Amyotrophic Lateral Sclerosis (ALS), Parkinson's Disease, Parkison's Disease with Lewy Bodies, Multiple Sclerosis, Huntington's Disease, and several sphingolipdidosis disorders (Lysosomal Storage Disorders) such as Niemann Pick Disease and Gauchers (Cutler et al, 2002, Ann Neurol, 52, 448-457; Haughey et al, 2004, Ann Neurol, 55, 257-267; Cutler et al, 2010, Neurol, 63, 636-630; Mielke et al, 2013, PLOS ONE, 8; Bras et al, 2008, FEBS Journal, 275, 5767-5773; Vidaurre et al, 2014, Brain, 137, 2271-2286; Fan et al, 2013, J Lipid Research, 54, 2800-2814). Modulating the activity of the S1P5 receptor in the central nervous system may be a therapeutic method for such neurodegenerative or cognitive disorders by shifting the ceramide/S1P balance towards S1P effects and away from ceramide-mediated cell death.

Soluble β-amyloid (Aβ) oligomers are considered the proximate effectors of synaptic injury and neuronal death occurring in AD. Aβ induces increased ceramide levels and oxidative stress in neuronal cultures, leading to apoptosis and cell death. S1P is a potent neuroprotective factor against this AB-induced damage, consistent with its role as ceramide's counterpart (Cutler et al, 2004, PNAS, 101, 2070-2075, Malaplate-Armand, 2006, Neurobiol. Dis, 23, 178-189). Aβ is also pro-inflammatory, inducing the migration of monocytes to sites of injury, and the S1P1, S1P3, S1P4, S1P5 agonist FTY720/Fingolimod inhibits such migration. Aβ is known to induce expression of S1P2 and S1P5, but not of S1P1, S1P3 and S1P4 (Kaneider et al, 2004, FASEB). The actions of FTY720/FIngolimod and those expressed by monocytes suggests these effects are mediated by the S1P5 receptor. The same applies to more recent findings that FTY720/Fingolimod is able to modulate AB-induced memory deficits (Fukumoto et al, 2014, Beh Brain Res, 268, 88-93).

Additional studies suggest a role for S1P in modulating pain signals. In example, S1P modulates action potentials in capsaicin-sensitive sensory neurons (Zhang et al, 2006, J Physiol, 575, 101-113) and S1P levels are known to be decreased in CSF in acute and inflammatory pain models (Coste et al, 2008, J Biol Chem, 283, 32442-32451). The S1P1, S1P3, S1P4, S1P5 receptor agonist FTY720/Fingolimod is indeed able to reduce nociceptive behavior in neuropathic pain models (Coste et al, 2008, 12, 995-1004), while the selective S1P1 agonist SEW2817 fails to have an effect. Given the high CNS expression of S1P5 and lack of effects of SP agonism, the effects can be contributed to effects on the S1P5 receptor.

In summary, potent and selective agents that are agonists of the S1P5 receptor will be beneficial for the treatment of cognitive disorders, neurodegenerative disorders and pain. In particular, S1P5-selective ligands would be beneficial for these diseases by not engaging the SP, S1P3 and/or S1P4 receptor ensuring a lack of peripheral immune suppression and cardiovascular side-effects.

Furthermore, it would be desirable to provide an S1P5 modulator that has full or nearly full agonistic properties towards the S1P5 receptor. A full agonist could be advantageous in indications where partial activation of S1P5 is insufficient.

WO 2011/017561 describes S1P agonists containing a fused cyclic core wherein optionally one the rings is a heterocycle. 1,2,3,4-substituted quinoline is mentioned among the possible cyclic cores. The 1,2,3,4-substituted quinoline core in WO 2011/017561 does not comprise a spiro-substituted cyclopropyl group to the piperidine ring of the quinoline core. The compounds therefore structurally different from the compounds of the present invention.

Currently, there is still a need for new, potent S1P receptor modulators, in particular selective S1P5 receptor modulators.

### SUMMARY OF THE INVENTION

It is an object of the present invention to provide S1P5 receptor modulators, in particular agonists, preferably to agonists with selectivity over S1P, S1P3 and/or S1P4 receptors to avoid unwanted cardiovascular and/or immunomodulatory effects. Furthermore, the S1P5 receptor modulator is preferably a full or a nearly full agonist towards the S1P5 receptor. It is a further objection of the invention to provide a method for treatment or alleviation of a variety of CNS disorders, such as cognitive disorders, in particular age-related cognitive decline. The invention therefore provides a compound based on a 1,2,3,4-substituted quinoline core comprising a cyclopropane ring that is spiro-substituted at the 7-position of the quinoline ring structure. More specifically, the invention provides a compound of formula (I):

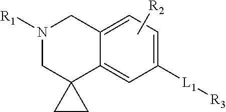

or a pharmaceutically acceptable salt, solvate, tautomer, stereoisomer or N-oxide thereof, wherein
R1 is selected from the group consisting of
—(C1-6)alkylene-R4 wherein one or more carbon atoms in the alkylene group, each independently, are optionally substituted with (CH₂)₂ to form a cyclopropyl moiety or with (CH₂)₃ to form a cyclobutyl moiety,
—(C3-6)cycloalkylene-R4
—(C1-3)alkylene-(C3-6)cycloalkylene-R4,
—(C3-6)cycloalkylene-(C1-3)alkylene-R4,
—(C1-3)alkylene-(C3-6)cycloalkylene-(C1-3)alkylene-R4,
wherein one or more carbon atoms in the alkylene and cycloalkylene groups, each independently, are optionally substituted with one or more halogen atoms; and
wherein R4 is selected from the group consisting of —COOH and —COO(C1-4)alkyl;
   R2 is hydrogen or R2 is one or more substituents independently selected from the group consisting of a halogen atom, (C1-4)alkyl optionally substituted with one or more halogen atoms, (C3-4)cycloalkyl optionally substituted with one or more halogen atoms, (C1-4)alkoxy optionally substituted with one or more halogen atoms, (C3-4)cycloalkoxy optionally substituted with one or more halogen atoms, and hydroxyl;
   L1 is selected from the group consisting of —C≡C—, —O—CH₂—, —CH₂—O—, —CH₂—CH₂—, —CH═CH—, —CF₂—CH₂—, —CH₂—CF₂—, —CHF—CH₂—, —CH₂—CHF—, —S—CH₂—, —CH₂—S—, —O— and —S—;
   R3 is a 5- to 6-membered ring, wherein the 5- to 6-membered ring is optionally substituted with one or more substituents, each independently selected from the group consisting of:
a halogen atom,
(C1-4)alkyl optionally substituted with one or more fluoro atoms,
(C3-6)cycloalkyl optionally substituted with a substituent selected from the group consisting of (C1-4)alkyl and a halogen atom, and
—(C1-3)alkylene-(C3-6)cycloalkyl optionally substituted with a substituent selected from the group consisting of (C1-4)alkyl and a halogen atom, and
(C1-4)alkoxy optionally substituted with one or more fluoro atoms,
(C3-6)cycloalkoxy optionally substituted with a substituent selected from the group consisting of (C1-4)alkyl and a halogen atom.
—S—(C1-4)alkyl,
—SF5,
*—(CH₂)_{q}—** wherein * and ** are attached to two adjacent C atoms in the 5- to 6-membered ring and q is an integer of 3 to 4,
*—CH₂—O—CH₂—CH₂—**, *—O—CH₂—CH₂—CH₂—**, *—CH₂—O—CH₂—O—** and *—O—CH₂—CH₂—O—**, wherein * and ** are attached to two adjacent C atoms in the 5- to 6-membered ring
*—O—CH₂—O—** or *—O—CH₂—CH₂—**, wherein * and ** are attached to two adjacent C atoms in the 5- to 6-membered ring.

In a further aspect the invention provides a pharmaceutical composition comprising a compound according to the invention or a pharmaceutically acceptable salt, solvate, tautomer, stereoisomer or N-oxide thereof and at least one pharmaceutically acceptable auxiliary.

In a still further aspect the invention provides a method of treatment, alleviation or prevention of a disease or condition in which an S1P receptor is involved or in which modulation of the endogenous S1P signaling system via an S1P receptor is involved, preferably S1P5, comprising administering to a patient in need thereof a compound according to the invention or a pharmaceutically acceptable salt, solvate, tautomer, stereoisomer or N-oxide thereof.

In a still further aspect the invention provides a use of a compound according to the invention or a pharmaceutically acceptable salt, solvate, tautomer, stereoisomer or N-oxide thereof for the manufacture of a medicament for the treatment, alleviation or prevention of a disease or condition in which an S1P receptor is involved or in which modulation of the endogenous S1P signaling system via an S1P receptor is involved, preferably S1P5 receptor.

In a still further aspect the invention provides a compound according to the invention or a pharmaceutically acceptable salt, solvate, tautomer, stereoisomer or N-oxide thereof for use in therapy.

In a still further aspect the invention provides a compound according to the invention or a pharmaceutically acceptable salt, solvate, tautomer, stereoisomer or N-oxide thereof for use in the treatment, alleviation or prevention of a disease or condition in which an S1P receptor is involved or in which modulation of the endogenous S1P signaling system via an S1P receptor is involved, preferably S1P5.

### DETAILED DESCRIPTION OF THE INVENTION

The compounds of the invention are modulators of the S1P receptor, in particular of the S1P5 receptor. More specifically, the compounds of the invention are S1P5 receptor agonists. The compound of the invention are preferably capable of acting as a full agonist towards a S1P5 receptor. The compounds of the invention and their pharmaceutically acceptable salts, solvates, tautomers, stereoisomers and N-oxides are in particular suitable for agonizing S1P5 in a subject suffering from a disorder in which modulation of S1P5 activity and the subsequent ceramide/S1P axis is beneficial. Administration of such compound to a subject is preferably such that S1P5 activity in the subject is altered and treatment is achieved. The compounds of the present invention are particularly suitable to treat, alleviate or prevent diseases and conditions in which an S1P receptor is involved or in which modulation of the endogenous S1P signaling system via an S1P receptor is involved. In particular the compounds of the present invention are suitable to treat, alleviate or prevent a disorder or condition selected from the group consisting of Alzheimer's Disease (AD) and associated dementia's, amyloid B-associated disorders, Mild Cognitive Impairment (MCI), Parkinson's Disease (PD), Lewy Body Dementia (LBD), Progressive Supranuclear Palsy (PSP), Cerebral Palsy (CP), Amyotrophic Lateral Sclerosis (ALS), Frontal Temporal Lobe Dementia (FTLD), multiple sclerosis, Huntington's Disease, neurological symptoms of sphingolipidosis disorders, a lysosomal storage disorder including Tay Sachs Disease, Sandhoff Disease, Fabry's Disease, Krabbe Disease, Gaucher's Disease, Niemann Pick A, B or C, and Batten's Disease, stroke, HIV-associated Dementia (HAD), HIV-associate Neurocognitive Disorder (HAND), HIV-associated neuropathy, schizophrenia, cognitive deficits in Schizophrenia, an attention deficit disorder including Anxiety Attention Deficit Disorder and Attention Deficit Hyperactivity Disorder (ADHD), a bipolar disorder, Obsessive-Compulsive Behavior, pain including neuropathic, back pain and pain-associated with multiple sclerosis, spinal cord injury, Parkinson's Disease, epilepsy, diabetes and cancer, cancer-induced peripheral neuropathy (CIPN), depression, treatment-resistant depression, Creutzfeld-Jakob Disease and other Prion-related Disorders, Down's Syndrome, autism, age-related cognitive decline or memory impairment, cognitive deficits associated with diabetes, dementia, dementia associated with Down's Syndrome, cognitive deficits in psychiatric disorders, dementia associated with Lewy Body pathology, diminished CNS function associated with traumatic brain injury, Pick's Disease, spinal cord injury, a demyelinating disorder, a disorder of basal ganglia and AIDS-associated dementia. Given the neuro-inflammatory actions of S1P receptors, and S1P5 in specific, as well as the peripheral localization of S1P5 in skin tissue and a role in endothelial function and NK cells, the compounds of the invention are further suitable to treat, alleviate or prevent a disease with a neuro-inflammatory component, in particular a disease or condition selected from the group consisting of Psoriasis type 1 and type 2, atopic dermatitis, dermatitis scleroderma, insulin-dependent diabetes mellitus, ulcerative colitis, atherosclerosis, sepsis syndrome, septic shock, Dengue hemorrhagic fever, Dengue, atopic allergy, HIV/AIDS, barrier-integrity associated lung diseases, leukemia, contact dermatitis, encephalomyelitis, Epstein Barr virus infection and other virus infections requiring cell-cell fusion.

In formula (I), R1 is selected from the group consisting of
—(C1-6)alkylene-R4 wherein one or more carbon atoms in the alkylene group, each independently, are optionally substituted with (CH₂)₂ to form a cyclopropyl moiety or with (CH₂)₃ to form a cyclobutyl moiety,
—(C3-6)cycloalkylene-R4,
—(C1-3)alkylene-(C3-6)cycloalkylene-R4,
—(C1-3)alkylene-(C3-6)cycloalkylene-(C1-3)alkylene-R4,
—(C3-6)cycloalkylene-(C1-3)alkylene-R4,
   wherein one or more carbon atoms in the alkylene and cycloalkylene groups, each independently, are optionally substituted with one or more halogen atoms; and wherein R4 is selected from the group consisting of —COOH and —COO(C1-4)alkyl.

R4 is preferably —COOH.

R1 may be selected from the group consisting of
—(C1-6)alkylene-R6,
—(C1-3)alkylene-R6, wherein one carbon atom in the alkylene group is substituted with (CH₂)₂ to form a cyclopropyl moiety or with (CH₂)₃ to form a cyclobutyl moiety,
—(C3-6)cycloalkylene-R6,
—(C1-3)alkylene-(C3-6)cycloalkylene-R4,
—(C3-6)cycloalkylene-(C1-3)alkylene-R6,
   wherein R6 is selected from the group consisting of —COOH and —COO(C1-4)alkyl, and wherein R6 is preferably —COOH.

Preferably, R1 is selected from the group consisting of: —CH₂—COOH, —(CH₂)₂—COOH, —(CH₂)₃—COOH, —(CH₂)₄—COOH, —(CH₂)₅—COOH, —CH(CH₃)—COOH, C(CH₃)₂—COOH, —CHCH₃—CH₂—COOH, —CH₂—CHCH₃—COOH, —CH₂—C(CH₃)₂—COOH, —C(CH₃)₂—CH₂—COOH, —CH₂—CHCH₃—CH₂—COOH, —CH₂—C(CH₃)₂—CH₂—COOH, —(CH₂)₂—CHCH₃—COOH, —(CH₂)₂—C(CH₃)₂—COOH, —C(CH₃)₂—(CH₂)₂—COOH, —CHCH₃—(CH₂)₂—COOH,

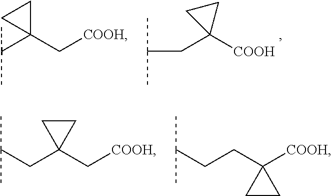

3-carboxycyclobutyl, 3-(carboxymethyl)cyclobutyl, (3-carboxycyclobutyl)methyl, 3-carboxy-3-methylcyclobutyl, 3-carboxycyclobutyl (C1-4)alkyl ester, 3-(carboxymethyl)cyclobutyl (C1-4)alkyl ester, (3-carboxycyclobutyl)methyl, 3-carboxy-3-methylcyclobutyl (C1-4)alkyl ester, —CH₂—COO(C1-4)alkyl, —(CH₂)₂—COO(C1-4)alkyl, —(CH₂)₃—COO(C1-4)alkyl, —CHCH₃—COO(C1-4)alkyl, —C(CH₃)₂—COO(C1-4)alkyl, —CHCH₃—CH₂—CH₂—COO(C1-4)alkyl, —CH₂—CHCH₃—CH₂—COO(C1-4)alkyl, —CH₂—CH₂—CHCH₃—COO(C1-4)alkyl, —C(CH₃)₂—CH₂—CH₂—COO(C1-4)alkyl, —CH₂—C(CH₃)₂—CH₂—COO(C1-4)alkyl, —CH₂—CH₂—C(CH₃)₂—COO(C1-4)alkyl, wherein for each of these R1 groups one or more carbon atoms, each independently, is optionally substituted with one or more F or Cl atoms. Preferably, F atoms are used for such substitutions.

More preferably, R1 is selected from the group consisting of —CH₂—COOH, —(CH₂)₂—COOH, —(CH₂)₃—COOH, —(CH₂)₄—COOH, —(CH₂)₅—COOH, —CH(CH₃)—COOH, —CHCH₃—CH₂—COOH, —CH₂—CHCH₃—COOH, —CH₂—C(CH₃)₂—COOH, —C(CH₃)₂—CH₂—COOH, —CH₂—CHCH₃—CH₂—COOH, —CH₂—C(CH₃)₂—CH₂—COOH, —(CH₂)₂—CHCH₃—COOH, —(CH₂)₂—C(CH₃)₂—COOH, —C(CH₃)₂—(CH₂)₂—COOH, —CHCH₃—(CH₂)₂—COOH,

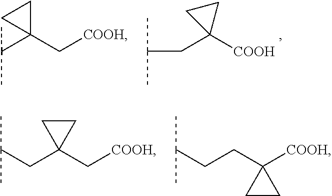

3-carboxycyclobutyl, 3-(carboxymethyl)cyclobutyl, (3-carboxycyclobutyl)methyl, 3-carboxy-3-methylcyclobutyl, 3-carboxycyclobutyl (C1-4)alkyl ester, 3-(carboxymethyl)cyclobutyl (C1-4)alkyl ester, (3-carboxycyclobutyl)methyl, 3-carboxy-3-methylcyclobutyl (C1-4)alkyl ester, —CH₂—COO(C1-4)alkyl, —(CH₂)₂—COO(C1-4)alkyl, —(CH₂)₃—COO(C1-4)alkyl, —CH₂—CHCH₃—CH₂—COO(C1-4)alkyl, —C(CH₃)₂—CH₂—CH₂—COO(C1-4)alkyl, —CH₂—C(CH₃)₂—CH₂—COO(C1-4)alkyl, —CH₂—CH₂—C(CH₃)₂—COO(C1-4)alkyl, —CH₂—CF₂—CH₂—COOH, —CH₂—CHF—CH₂—COOH, —CH₂—CF(CH₃)—CH₂—COOH. Optionally, R1 can be selected from the above group, wherein for each of these R1 groups one or more carbon atoms, each independently, is optionally substituted with one or more F or Cl atoms.

Even more preferably, R1 is selected from the group consisting of —CH₂—COOH, —CHCH₃—COOH, —(CH₂)₂—COOH, —(CH₂)₃—COOH, —CH₂—CHCH₃—COOH, —CHCH₃—CH₂—COOH, —CH₂—CHCH₃—CH₂—COOH, —CH₂—C(CH₃)₂—CH₂—COOH, —(CH₂)₂—CHCH₃—COOH, —CHCH₃—(CH₂)₂—COOH, —(CH₂)₂—C(CH₃)₂—COOH,

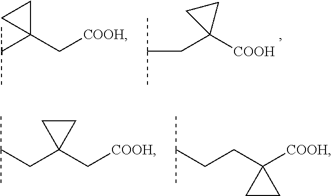

3-carboxycyclobutyl, 3-(carboxymethyl)cyclobutyl, —CH₂—CF₂—CH₂—COOH, —CH₂—CHF—CH₂—COOH, and —CH₂—CF(CH₃)—CH₂—COOH.

Even more preferably, R1 is selected from the group consisting of —CH₂—COOH, —CH₂—CH₂—COOH, —CH₂—CH₂—CH₂—COOH, —CH₂—CH(CH₃)—CH₂—COOH, —CH₂—CHF—CH₂—COOH, —CH₂—CF₂—CH₂—COOH, —CH₂—CF(CH₃)—CH₂—COOH and 3-carboxycyclobutyl.

In another preferred embodiment R1 is selected from —(C1-4)alkylene-COOH and -1,3-cyclobutylene-COOH. In particular, most preferred is when R1 is selected from the group consisting of —(CH₂)—COOH, —(CH₂)₂—COOH, —(CH₂)₃—COOH, —CH₂—CHCH₃—CH₂—COOH and -1,3-cyclobutylene-COOH.

In one embodiment, R1 is selected from the group consisting of —CH₂—COOH, —(CH₂)₂—COOH, —(CH₂)₃—COOH, —CH₂—CHCH₃—CH₂—COOH and -1,3-cyclobutylene-COOH.

In one embodiment, R1 is -1,3-cyclobutylene-COOH.

In one embodiment, R1 is selected from the group consisting of —CH₂—CF₂—CH₂—COOH, —CH₂—CHF—CH₂—COOH, and —CH₂—CF(CH₃)—CH₂—COOH.

R2 is hydrogen or R2 is one or more substituents independently selected from the group consisting of a halogen atom, (C1-4)alkyl optionally substituted with one or more halogen atoms, (C3-4)cycloalkyl optionally substituted with one or more halogen atoms, (C1-4)alkoxy optionally substituted with one or more halogen atoms, (C3-4)cycloalkoxy optionally substituted with one or more halogen atoms, and hydroxyl.

Preferably, R2 is selected from the group consisting of fluoro, chloro, methyl optionally substituted with one or more halogen atoms, ethyl optionally substituted with one or more halogen atoms, propyl optionally substituted with one or more halogen atoms, cyclopropyl optionally substituted with one or more halogent atoms and cyclobutyl optionally substituted with one or more halogen atoms. In case of a substitution with a halogen atom, preferably fluor or chloro atoms are used, even more preferably fluoro atoms.

Even more preferably, R2 is hydrogen or R2 is one substituent selected from the group consisting of —F, —Cl, —CH₃, —CF₃, —CH₂—CH₃, —CH₂—CH₂—CH₃, —CH(CH₃)—CH₃, —CH(CF₃)—CH₃, cyclopropyl, cyclobutyl.

In an embodiment, R2 is hydrogen. This means that no R2 group is substituted to the quinoline core.

L1 is selected from the group consisting of —C≡C—, —O—CH₂—, —CH₂—O—, —CH₂—CH₂—, —CH═CH—, —CF₂—CH₂—, —CH₂—CF₂—, —CHF—CH₂—, —CH₂—CHF—, —S—CH₂—, —CH₂—S—, —O— and —S—

More preferably, L1 is selected from the group consisting of —C≡C—, —O—CH₂—, —CH₂—O—, —CH₂—CH₂—, —CH═CH—, —CF₂—CH₂—, —CH₂—CF₂—, —CHF—CH₂—, —CH₂—CHF—, —S—CH₂— and —CH₂—S—;

Even more preferably, L1 is selected from the group consisting of —C≡C—, —O—CH₂— and —O—CH₂—.

Even more preferably, L1 is selected from the group consisting of —C≡C— and O—CH₂— with C being attached to R3. Even more preferably, L1 is —C≡C—.

R3 is a 5- to 6-membered ring, i.e. a 5-membered ring or a 6-membered ring. The ring may optionally be substituted as described below. R3 may be a saturated or an unsaturated ring. Preferably, R3 is an unsaturated ring, more preferably an aromatic ring. Further, R3 may be a heterocyclic ring. R3 may comprise 0, 1, 2 or 3 heteroatoms in its ring structure (i.e. not including any substituents on the ring). Preferably, the heteroatoms are independently selected from O, N and S, more preferably from N and S, and even more preferably is N. In case of a heterocyclic ring, R3 preferably comprises one heteroatom.

The 5- to 6-membered ring may be selected from the group consisting of unsaturated 6-membered rings, unsaturated 5-membered rings, saturated 6-membered rings and saturated 5-membered rings.

Preferably, the 5- to 6-membered ring is selected from the group consisting of phenyl, unsaturated 5-membered rings with a single heteroatom, unsaturated 5-membered rings with two heteroatoms, unsaturated 6-membered rings with a single heteroatom and unsaturated 6-membered rings with two heteroatoms. R3 may also be a saturated 5- to 6-membered ring.

More in particular, the 5- to 6-membered ring is selected from the group consisting of:
phenyl, optionally substituted with phenyl;
an unsaturated 5-membered ring with a single heteroatom selected from the group consisting of thiophenyl, furan and pyrrolyl;
an unsaturated 5-membered ring with two heteroatoms selected from the group consisting of pyrazolyl, imidazolyl, oxazole, thiazolyl and isothiazolyl;
an unsaturated 6-membered ring with a single heteroatom selected from the group consisting of pyridinyl;
an unsaturated 6-membered ring with two heteroatoms selected from pyrimidinyl, pyrazinyl and pyridazinyl;
a saturated 5-6 membered ring selected from the group consisting of cyclopentyl, cyclohexyl, tetrahydropyranyl and tetrayhydrofuranyl.

Preferably, R3 is a 5- to 6-membered ring selected from the group consisting of phenyl, pyridinyl, thiophenyl, pyrazolyl, thiazolyl, pyrimidinyl, pyrazinyl, pyridazinyl, cyclopentyl and cyclohexyl

The 5- to 6-membered ring may be substituted with 0, 1, 2 or 3 substituents. Preferably, the 5- to 6-membered ring is substituted with at least one substituent. More preferably, the 5- to 6-membered ring is substituted with one or two substituents. The substituents may be the same or different from each other. The substituents are preferably independently selected from the group of preferred R3 substituents or the group of particularly preferred R3 substituents described above.

In case the 5- to 6-membered ring is an unsaturated ring, the unsaturated ring is preferably selected from the group of phenyl, pyridinyl, thiophenyl, pyrazolyl, thiazolyl, pyrimidinyl, pyrazinyl and pyridazinyl. Even more preferably, the 5- to 6-membered ring is selected from the group consisting of phenyl, pyridinyl and thiophenyl. The substituents are preferably independently selected from the group of preferred R3 substituents or the group of particularly preferred R3 substituents described below.

In case the 5- to 6-membered ring is a saturated ring, the saturated ring is preferably selected from the group consisting of cycloalkyls consisting of cyclopentyl and cyclohexyl. The substituents are preferably independently selected from the group of preferred R3 substituents or the group of particularly preferred R3 substituents described below.

In preferred compounds, the 5- to 6-membered ring of R3 is an unsaturated 6-membered ring, for example phenyl or pyridinyl, or when the 5- to 6-membered ring is an unsaturated 5-membered ring, for example thiophenyl.

The 5- to 6-membered ring of R3 is optionally substituted with one or more substituents, each independently selected from the group consisting of:
phenyl, provided that the 5- to 6-membered ring is also phenyl
a halogen atom,
(C1-4)alkyl optionally substituted with one or more fluoro atoms,
(C3-6)cycloalkyl optionally substituted with a substituent selected from the group consisting of (C1-4)alkyl and a halogen atom, and
—(C1-3)alkylene-(C3-6)cycloalkyl optionally substituted with a substituent selected from the group consisting of (C1-4)alkyl and a halogen atom, and
(C1-4)alkoxy optionally substituted with one or more fluoro atoms,
(C3-6)cycloalkoxy optionally substituted with a substituent selected from the group consisting of (C1-4)alkyl and a halogen atom.
—S—(C1-4)alkyl,
—SF5,
*—(CH₂)_{q}—** wherein * and ** are attached to two adjacent C atoms in the 5- to 6-membered ring and q is an integer of 3 to 4,
*—CH₂—O—CH₂—CH₂—**, *—O—CH₂—CH₂—CH₂—**, *—CH₂O—CH₂—O—** or *—O—CH₂—CH₂—O—**wherein * and ** are attached to two adjacent C atoms in the 5- to 6-membered ring
*—O—CH₂—O—** or *—O—CH₂—CH₂—**, wherein * and ** are attached to two adjacent C atoms in the 5- to 6-membered ring.

The halogen atom may be chloro or fluoro.

The (C1-4)alkyl optionally substituted with one or more fluoro atoms may be a (C1-4)alkyl selected from the group consisting of CHs, —CH₂—CH₃, —CHCH₃—CHs, —C(CH₃)₂—CH₃, —(CH₂)₂—CH₃, —CH₂—CHCH₃—CH₃, —CHCH₃—CH₂—CH₃, —CH₂—C(CH₃)₂—CH₃, which (C1-4)alkyl may be substituted with one or more fluoro atoms. —C(CH₃)₂—CH₂—CH₃ group, such as —CF₃.

The (C3-6)cycloalkyl may be a ring selected from the group consisting of cyclopropyl, cyclobutyl and cyclopentyl.

The (C1-4)alkoxy may be selected from —O—CH₃, —O—CH₂—CH₃, —O—CHCH₃—CH₃, —O—(CH₂)₂—CH₃, —O—CH₂—CHCH₃—CH₃, —O—CHCH₃—CH₂—CH₃ and —O—C(CH₃)₃.

The (C3-6)cycloalkoxy may be selected from the group consisting of —O— cyclopentyl, O-cyclobutyl, O-cyclopropyl.

The —(C1-3)alkylene-(C3-6)cycloalkyl may be selected from —(CH₂)ₚ-cyclopropyl, —(CH₂)ₚ-cyclobutyl and —(CH₂)ₚ-cyclopentyl, wherein p is an integer from 0 to 3.

Preferably, the 5- to 6-membered ring of R3 is optionally substituted with one or more substituents that are independently selected from the group of preferred R3 substituents consisting of:
—F, —Cl,
(C1-4)alkyl optionally substituted with one or more fluoro atoms,
—CF₃, —O—CF₃
(C3-5)cycloalkyl optionally substituted with a substituent selected from the group consisting of (C1-4)alkyl, fluoro and chloro,
(C1-4)alkoxy optionally substituted with one or more fluoro atoms,
(C3-6)cycloalkoxy optionally substituted with a substituent selected from the group consisting of (C1-4)alkyl, fluoro and chloro,
—S—CH₃, —S—CH₂—CH₃, —S—CHCH₃—CHs
*—(CH₂)_{q}—** wherein * and ** are attached to two adjacent C atoms in the 5- to 6-membered ring and q is an integer of 3 to 4,
*—CH₂—O—CH₂—CH₂—**, *—O—CH₂—CH₂—CH₂—**, *—CH₂—O—CH₂—O—** or *—O—CH₂—CH₂—O—**wherein * and ** are attached to two adjacent C atoms in the unsaturated ring
*—O—CH₂—O—** or *—O—CH₂—CH₂—**, wherein * and ** are attached to two adjacent C atoms in the unsaturated ring.
   Additionally, R3 may also be substituted with one of the the above substituents, wherein one or more carbon atoms in the substituents are independently substituted with F or Cl.

Even more preferably, the 5- to 6-membered ring of R3 is optionally substituted with one or more substituents that are independently selected from the group of particularly preferred R3 substituents consisting of
—F, —Cl,
—CH₃, —CH₂—CH₃, —CHCH₃—CH₃, —C(CH₃)₂—CH₃, —(CH₂)₂—CH₃, —CH₂—CHCH₃—CH₃, —CHCH₃—CH₂—CH₃, —CH₂—C(CH₃)₂—CH₃, —C(CH₃)₂—CH₂—CH₃,
—CF₃, —O—CF₃
-cyclopropyl, -cyclybutyl, -cyclopentyl,
—(CH₂)ₚ-cyclopropyl, —(CH₂)ₚ-cyclobutyl and —(CH₂)ₚ-cyclopentyl, wherein p is an integer from 0 to 3,
—O—CH₃, —O—CH₂—CH₃, —O—CHCH₃—CH₃, —O—(CH₂)₂—CH₃, —O—CH₂—CHCH₃—CH₃, —O—CHCH₃—CH₂—CH₃, —O—C(CH₃)₃,
—O-cyclopentyl, O-cyclobutyl, O-cyclopropyl.

In one embodiment, R3 is indanyl. Indanyl is a phenyl ring substituted with *—(CH₂)₃—** wherein * and ** are attached to two adjacent C atoms in the phenyl.

Particularly preferred combinations of the parameters R1, R2, R3 and L1 are as follows.

Preferably, L1 is selected from the group consisting of —C≡C—, —O—CH₂— and —CH₂—O—; while R3 is a 5- or 6-membered ring selected from the group consisting of phenyl, pyridinyl, thiophenyl, pyrazolyl, thiazolyl, pyrimidinyl, pyrazinyl, pyridazinyl, cyclopentyl and cyclohexyl. The 5- or 6-membered ring ring may optionally be substituted with 1, 2 or 3 substituents. The possible substitutents are described in detail above.

In one embodiment, L1 is selected from the group consisting of —C≡C—, —O—CH₂— and —CH₂—O—; while R3 is an unsaturated 5- or 6-membered ring preferably selected from the group consisting of phenyl, pyridinyl, thiophenyl, pyrazolyl, thiazolyl, pyrimidinyl, pyrazinyl, pyridazinyl; for example selected from the group consisting of phenyl, pyridinyl and thiophenyl. The unsaturated ring may optionally be substituted with 1, 2 or 3 substituents. The possible substitutents are described in detail above.

In one embodiment, L1 is selected from the group consisting of —C≡C—, —O—CH₂— and —CH₂—O—; while R3 is a saturated 5- or 6-membered ring preferably selected from cycloalkyl and cyclohexyl. The saturated ring may optionally be substituted with 1, 2 or 3 substituents. The possible substitutents are described in detail above.

Even more preferably, L1 is —C≡C—; while R3 is a 5- or 6-membered ring selected from the group consisting of phenyl, pyridinyl, thiophenyl, pyrazolyl, thiazolyl, pyrimidinyl, pyrazinyl, pyridazinyl, cyclopentyl and cyclohexyl. The 5- or 6-membered ring ring may optionally be substituted with 1, 2 or 3 substituents. The possible substitutents are described in detail above.

In the preferred combinations and preferred embodiments described above for L1/R3, R2 is preferably absent or selected from the group consisting of —F, —Cl, —CH₃, —CF₃, —CH₂—CH₃, —CH₂—CH₂—CH₃, —CH(CH₃)—CH₃, —CH(CF₃)—CH₃, cyclopropyl, cyclobutyl; and is even more preferably absent. In this case, R1 is preferably one of the preferred R1 groups described above.

The compound according to the invention may further be a compound according to formula (I) or a pharmaceutically acceptable salt, solvate, tautomer, stereoisomer or N-oxide thereof, wherein

R1 is selected from the group consisting of
—(C1-6)alkylene-R6,
—(C1-3)alkylene-R6, wherein one carbon atom in the alkylene group is substituted with (CH₂)₂ to form a cyclopropyl moiety or with (CH₂)₃ to form a cyclobutyl moiety,
—(C3-6)cycloalkylene-R6,
—(C1-3)alkylene-(C3-6)cycloalkylene-R4,
—(C3-6)cycloalkylene-(C1-3)alkylene-R6, wherein R6 is selected from the group consisting of —COOH and —COO(C1-4)alkyl, and wherein R6 is preferably —COOH;

R2 is hydrogen or R2 is selected from the group consisting of fluoro, chloro, methyl optionally substituted with one or more halogen atoms, ethyl optionally substituted with one or more halogen atoms, propyl optionally substituted with one or more halogen atoms, cyclopropyl optionally substituted with one or more halogent atoms and cyclobutyl optionally substituted with one or more halogen atoms; wherein R2 is preferably hydrogen or R2 is one substituent selected from the group consisting of —F, —Cl, —CH₃, —CF₃, —CH₂—CH₃, —CH₂—CH₂—CH₃, —CH(CH₃)—CH₃, —CH(CF₃)—CH₃, cyclopropyl, cyclobutyl;

L1 is selected from the group consisting of —C≡C—, —O—CH₂— and —CH₂—O— and is preferably —C≡C—;

R3 is a 5- or 6-membered ring selected from the group consisting of phenyl, pyridinyl, thiophenyl, pyrazolyl, thiazolyl, pyrimidinyl, pyrazinyl, pyridazinyl, cyclopentyl and cyclohexyl, wherein the ring is optionally substituted with 1, 2 or 3 substituents, wherein the substituent is selected from the group consisting of preferred substitutents or particularly preferred substituents described above.

The preferred groups for R1 described above also apply to this particular embodiment.

Preferred compounds further include:
2-(6′-((2-chloro-6-ethylbenzyl)oxy)-1′H-spiro[cyclopropane-1,4′-isoquinolin]-2′(3′H)-yl)acetic acid;
3-(6′-((2,3-difluorophenyl)ethynyl)-1′H-spiro[cyclopropane-1,4′-isoquinolin]-2′(3′H)-yl)propanoic acid;
3-(6′-((2,6-dichlorophenyl)ethynyl)-1′H-spiro[cyclopropane-1,4′-isoquinolin]-2′(3′H)-yl)propanoic acid;
3-(6′-((4-ethoxyphenyl)ethynyl)-1′H-spiro[cyclopropane-1,4′-isoquinolin]-2′(3′H)-yl)propanoic acid;
3-(6′-((4-isopropylphenyl)ethynyl)-1′H-spiro[cyclopropane-1,4′-isoquinolin]-2′(3′H)-yl)propanoic acid;
3-(6′-((5-isopropoxypyridin-2-yl)ethynyl)-1′H-spiro[cyclopropane-1,4′-isoquinolin]-2′(3′H)-yl)cyclobutanecarboxylic acid;
3-(6′-(thiophen-3-ylethynyl)-1′H-spiro[cyclopropane-1,4′-isoquinolin]-2′(3′H)-yl)propanoic acid;
3-methyl-4-(6′-(thiophen-3-ylethynyl)-1′H-spiro[cyclopropane-1,4′-isoquinolin]-2′(3′H)-yl)butanoic acid;
4-(6′-((2,6-dichlorobenzyl)oxy)-1′H-spiro[cyclopropane-1,4′-isoquinolin]-2′(3′H)-yl)butanoic acid;
4-(6′-((2-(trifluoromethoxy)benzyl)oxy)-1′H-spiro[cyclopropane-1,4′-isoquinolin]-2′(3′H)-yl)butanoic acid;
4-(6′-((2-chloro-6-ethylbenzyl)oxy)-1′H-spiro[cyclopropane-1,4′-isoquinolin]-2′(3′H)-yl)butanoic acid;
4-(6′-((2-methylbenzyl)oxy)-1′H-spiro[cyclopropane-1,4′-isoquinolin]-2′(3′H)-yl)butanoic acid;
4-(6′-((3-fluoro-4-(trifluoromethoxy)phenyl)ethynyl)-1′H-spiro[cyclopropane-1,4′-isoquinolin]-2′(3′H)-yl)-3-methylbutanoic acid;
4-(6′-((3-fluoro-4-(trifluoromethoxy)phenyl)ethynyl)-1′H-spiro[cyclopropane-1,4′-isoquinolin]-2′(3′H)-yl)butanoic acid;
4-(6′-((3-fluorobenzyl)oxy)-1′H-spiro[cyclopropane-1,4′-isoquinolin]-2′(3′H)-yl)butanoic acid;
4-(6′-((4-(cyclopentyloxy)phenyl)ethynyl)-1′H-spiro[cyclopropane-1,4′-isoquinolin]-2′(3′H)-yl)butanoic acid;
4-(6′-((4-(trifluoromethoxy)phenyl)ethynyl)-1′H-spiro[cyclopropane-1,4′-isoquinolin]-2′(3′H)-yl)butanoic acid;
4-(6′-((4-ethoxy-3-fluorophenyl)ethynyl)-1′H-spiro[cyclopropane-1,4′-isoquinolin]-2′(3′H)-yl)-3-methylbutanoic acid;
4-(6′-((4-isopropoxyphenyl)ethynyl)-1′H-spiro[cyclopropane-1,4′-isoquinolin]-2′(3′H)-yl)-3-methylbutanoic acid;
4-(6′-((4-isopropoxyphenyl)ethynyl)-1′H-spiro[cyclopropane-1,4′-isoquinolin]-2′(3′H)-yl)butanoic acid;
4-(6′-((4-isopropylphenyl)ethynyl)-1′H-spiro[cyclopropane-1,4′-isoquinolin]-2′(3′H)-yl)-3-methylbutanoic acid;
4-(6′-((6-ethoxy-4-methylpyridin-3-yl)ethynyl)-1′H-spiro[cyclopropane-1,4′-isoquinolin]-2′(3′H)-yl)-3-methylbutanoic acid;
4-(6′-((6-isopropoxypyridin-3-yl)ethynyl)-1′H-spiro[cyclopropane-1,4′-isoquinolin]-2′(3′H)-yl)-3-methylbutanoic acid; and
4-(6′-((6-isopropoxypyridin-3-yl)ethynyl)-1′H-spiro[cyclopropane-1,4′-isoquinolin]-2′(3′H)-yl)butanoic acid.

Without wishing to be bound by any theory, the inventors believe that the spiro-linked cyclopropanyl moiety may have a positive effect on the compound's agonistic potency and efficacy towards S1P5 receptors, as measured e.g. by the EC50 and Emax in cells expressing the human S1P5 receptors.

Further particularly preferred compounds of the invention are depicted in table 1.

Particularly preferred compounds depicted in table 1 are compounds having a EC50 for the S1P5 receptor of 100 nM or less, as shown in table 1, i.e. compounds having an S1P5 EC50 range of A, B or E in table 1. Further particularly preferred compounds depicted in table 1 are compounds having a EC50 for the S1P5 receptor of 10 nM or less, as shown in table 1, i.e. compounds having an S1P5 EC50 range of A in table 1. Such compounds further preferably have an EC50 for at least one of the S1P1 receptor, the S1P3 receptor and the S1P4 receptor of more than 1 μM as shown in table 1, preferably an EC50 of more than 1 μM as shown in table 1 for the S1P1 receptor and/or the S1P3 receptor.

Furthermore, particularly preferred compounds depicted in table 1 are compounds having a hS1P5 Emax of at least 100.

Hence, in a particularly preferred embodiment are provided compounds depicted in table 1 having an S1P5 EC50 of 100 nM or less (indicated with range A, B or E in table 1) and EC50 of more than 1 μM for at least one of the S1P1 receptor, the S1P3 receptor and the S1P4 receptor, preferably an EC50 of more than 1 μM as shown in table 1 for the S1P1 receptor and/or the S1P3 receptor. In a further particularly preferred embodiment are provided compounds depicted in table 1 having an S1P5 EC50 of 10 nM or less (indicated with range A in table 1) and EC50 of more than 1 μM for at least one of the S1P1 receptor, the S1P3 receptor and the S1P4 receptor, preferably an EC50 of more than 1 μM as shown in table 1 for the S1P1 receptor and/or the S1P3 receptor. Even more preferably, such compounds also have a hS1P5 Emax of at least 100.

Furthermore, in another particularly preferred embodiment are provided compounds depicted in table 1 having a hS1P5 Emax of at least 100 and an S1P5 EC50 of 100 nM or less (indicated with range A, B or E in table 1), preferably in combination with an EC50 of more than 1 μM for at least one of the S1P1 receptor, the S1P3 receptor and the S1P4 receptor, more preferably an EC50 of more than 1 μM as shown in table 1 for the S1P1 receptor and/or the S1P3 receptor.

As used herein, the term “a halogen atom” refers to fluoro, chloro, bromo, or iodo. Preferred halogen atoms are fluoro and chloro.

As used herein, the term “(Cx-y)alkyl” refers to a branched or unbranched alkyl group having x-y carbon atoms. For instance, (C1-4)alkyl means a branched or unbranched alkyl group having 1-4 carbon atoms, for example methyl, ethyl, propyl, isopropyl and butyl. Similarly, the term “(C1-2) alkyl” refers to an alkyl group having 1 or 2 carbon atoms. Preferred alkyl groups are methyl and ethyl.

As used herein, the term (Cx-y)alkoxy refers to an alkoxy group having x-y carbon atoms, wherein the alkyl moiety is as defined above. For instance, the term (C1-4)alkoxy means an alkoxy group having 1-4 carbon atoms. Preferred alkoxy groups are methoxy and ethoxy.

As used herein, the term “(Cx-y)alkylene” refers to a branched or unbranched saturated alkylene group having x-y carbon atoms. For instance, the term “(C1-4)alkylene” means a saturated alkylene group having 1-4 carbon atoms, for example methylene, (CH₂)₃—CHCH₃—, —C(CH₃)₂—, —CHCH₃CH₂—. As another example, the term “(C1-6)alkylene means a saturated alkylene group having 1-6 carbon atoms”. In the definition of R1 as —(C1-6)alkylene-R4, one or more carbon atoms in the alkylene group may independently be substituted with (CH₂)₂ to form a cyclopropyl moiety, for instance to form an R1 group

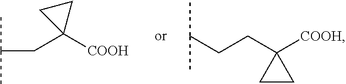

or with (CH₂)₃ to form a cyclobutyl moiety.

As used herein a dashed line in a partial structure, such as

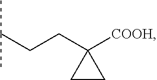

means that the partial structure is attached to the remainder of the structure at the site of the dashed line. For instance, if R1 is

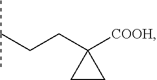

the compound of formula (I) is

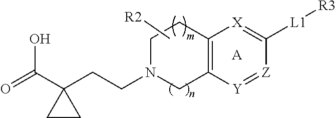

As used herein the term “(Cx-y)alkenyl” means a branched or unbranched alkenyl group having x-y carbon atoms, wherein the double bond may be present at various positions in the group. Examples are ethenyl, propenyl, 1-butenyl, 2-butenyl. For instance, the term “(C2-4)alkenyl” means a branched or unbranched alkenyl group having 2-4 carbon atoms.

As used herein, the term “(Cx-y)alkynyl” refers to a branched or unbranched alkynyl group having x-y carbon atoms, wherein the triple bond may be present at different positions in the group, for example ethynyl, propanyl, 1-butynyl, 2-butynyl. For instance, the term “(C2-4)alkynyl” refers to a branched or unbranched alkynyl group having 2-4 carbon atoms.

As used herein the term “(Cx-y)cycloalkyl” refers to a cyclic alkyl group having x-y carbon atoms. For instance, the term “(C3-6)cycloalkyl” refers to a cyclic alkyl group having 3-6 carbon atoms, i.e. cyclopropyl, cyclobutyl, cyclopentyl or cyclohexyl.

As used herein the term “(Cx-y)cycloalkenyl” means a cyclic alkenyl group having x-y carbon atoms. For instance, the term “(C4-6)cycloalkenyl” means a cyclic alkenyl group having 4-6 carbon atoms and comprising one or two double bonds, for example cyclohexenyl. Preferably a cycloalkenyl as used herein has one carbon-carbon double bond, e.g. cyclobutene, cyclopentene, cyclohexene and cycloheptene.

As used herein the term “(Cx-y)cycloalkylene” means a saturated cyclic group having x-y carbon atoms. For instance, the term “(C3-7)cycloalkylene” means a saturated cyclic group having 3-7 carbon atoms, e.g. cyclobutylene, cyclopentylene, cyclohexylene and cycloheptane.

As used herein, the term “5- or 6-membered ring” means a cyclic group having a ring that consists of 5 or 6 members (excluding any substituents on the ring, such as hydrogen, halogen, etc.). The members may all be carbon atoms. Alternatively, the ring may be heterocyclic, in which case the members may in addition to carbon atoms also comprise one or more members of oxygen, nitrogen or sulfur atoms. The ring may be saturated or unsaturated. The ring may be aromatic or non-aromatic. Preferred 5- to 6-membered rings may contain up to two heteroatoms, preferably O, S or N. The 5- to 6-membered ring may be selected from the group consisting of unsaturated 6-membered rings optionally comprising 1-3 heteroatoms, unsaturated 5-membered rings optionally comprising 1-3 heteroatoms, saturated 6-membered rings optionally comprising 1-3 heteroatoms and saturated 5-membered rings optionally comprising 1-3 heteroatoms. Examples of 5- to 6-membered rings are tetrahydrofuran, furan, 1,3-dioxalane, tetrahydrothiphene, thiphene, oxazole, isoxazole, isothiazole, thiazole, oxathiolane, oxadiazole, thiadiazole, piperidine, pyridine, piperazine, pyridazine, pyrimidine, pyrazine, triazine, tetrahydropyran, pyran, 1,4-dioxane, 1,4-dioxine, thiane, thiopyran, dithiane, trithiane, morpholine, oxazine, thiomorpholine and thiazine. Preferred aromatic 5- to 6-membered rings are phenyl, thiophenyl, furan and pyrrolyl, pyrazolyl, imidazolyl, oxazole, thiazolyl, isothiazolyl, pyridinyl, pyrimidinyl, pyrazinyl and pyridazinyl. Preferred saturated 6-membered rings are cyclopentyl, cyclohexyl, tetrahydropyranyl and tetrayhydrofuranyl.

With respect to substituents, the term “optionally substituted” indicates a group may be unsubstituted or substituted with the indicated number and type of the substituent(s).

The term “independently substituted” means that if a group that is substituted with more than one substituent, these substituents may be the same or different from each other. Similarly, if multiple atoms have one substituent selected from a group of substituents, the term “independently substituted” means that each atom has a substituent that may be the same or different from the substituent of the other atom(s).

The compounds of the present invention may contain one or more asymmetric centers and can thus occur as racemates and racemic mixtures, single enantiomers, diastereomeric mixtures and individual diastereomers. Additional asymmetric centers may be present depending upon the nature of the various substituents on the compound. Each such asymmetric center will independently produce two optical isomers and it is intended that all of the possible optical isomers and diastereomers in mixtures and as pure or partially purified compounds are included within the ambit of this invention. The present invention encompasses all such isomeric forms of these compounds. The independent syntheses of these diastereomers or their chromatographic separations may be achieved with any method known in the art, for instance as described in the Examples. The absolute stereochemistry of a compound may be determined by the x-ray crystallography of crystalline products or crystalline intermediates which are derivatized, if necessary, with a reagent containing an asymmetric center of known absolute configuration. If desired, racemic mixtures of the compounds may be separated so that the individual enantiomers are isolated. The separation can be carried out by methods well known in the art, such as chiral HPLC or SFC (Supercritical Fluid Chromatography) techniques. In the Examples, two suitable SFC methods are described.

Salts of compounds according to the invention are also provided. Such salts include, but are not limited to, acid addition salts and base addition salts. The term “pharmaceutically acceptable salt” as used herein refers to those salts retain the pharmacological activity of the compounds and that are, within the scope of sound medical judgment, suitable for use in humans or animals without undue toxicity, irritation, allergic response, and the like, and are commensurate with a reasonable benefit/risk ratio. Pharmaceutically acceptable salts are well-known in the art. They can be prepared in situ when isolating and purifying the compounds of the invention, or separately by reacting them with pharmaceutically acceptable non-toxic bases or acids, including inorganic or organic bases and inorganic or organic acids, for instance by reacting the free acid or free base forms of the product with one or more equivalents of the appropriate acid or base in a solvent or medium in which the salt is insoluble, or in a solvent such as water or an organic solvent which is then removed in vacuo or by freeze-drying, or by exchanging the cations of an existing salt for another cation on a suitable ion exchange resin. Examples of pharmaceutically acceptable acids and bases include organic and inorganic acids such as acetic acid, propionic acid, lactic acid, glycolic acid, oxalic acid, pyruvic acid, succinic acid, maleic acid, malonic acid, trifluoroacetic acid, cinnamic acid, sulfuric acid, hydrochloric acid, hydrobromic acid, nitric acid, perchloric acid and phosphoric acid, and bases such as ethylamine, methylamine, dimethylamine, triethylamine, isopropylamine, diisopropylamine, and other mono-, di- and trialkylamines, and arylamines.

Compounds may exist as polymorphs and as such are intended to be included in the present invention. In addition, compounds may form solvates with water (i.e., hydrates) or common organic solvents, and such solvates are also intended to be encompassed within the scope of this invention. Hydrates are a preferred type of solvate.

Isotopically-labeled compounds of formula (I) or pharmaceutically acceptable salts thereof, including compounds of formula (I) isotopically-labeled to be detectable by PET or SPECT, also fall within the scope of the invention. The same applies to compounds of formula (I) labeled with [13C]—, [14C]—, [3H]—, [18F]—, [125I]— or other isotopically enriched atoms, suitable for receptor binding or metabolism studies.

The compounds of the invention may be prepared by methods known in the art and to a skilled person. Suitable methods to prepare the compounds are described in the experimental section of this description.

Compounds according to the invention are useful in counteracting diseases or disorders mediated by an S1P receptor, preferably S1P5. They are preferably mixed with pharmaceutically suitable auxiliaries, e.g. as described in the standard reference “Remington, The Science and Practice of Pharmacy” (21st edition, Lippincott Williams & Wilkins, 2005, see especially Part 5: Pharmaceutical Manufacturing). The compounds together with pharmaceutically suitable auxiliaries may be compressed into solid dosage units, such as pills or tablets, or be processed into capsules or suppositories. By means of pharmaceutically suitable liquids the compounds can also be applied in the form of a solution, suspension or emulsion.

Provided is therefore a pharmaceutical composition comprising a compound according to the invention or a pharmaceutically acceptable salt, solvate, tautomer, stereoisomer or N-oxide thereof, and at least one pharmaceutically acceptable carrier, diluent and/or excipient. By “pharmaceutically acceptable” it is meant that the carrier, diluent or excipient must be compatible with the other ingredients of the formulation and not deleterious to the recipient thereof. In general, any pharmaceutically suitable additive which does not interfere with the function of the active compounds can be used. A pharmaceutical composition according to the invention is preferably suitable for human use.

Examples of suitable carriers comprise a solution, lactose, starch, cellulose derivatives and the like, or mixtures thereof. In a preferred embodiment said suitable carrier is a solution, for example saline. For making dosage units, e.g. tablets, the use of conventional additives such as fillers, colorants, polymeric binders and the like, is contemplated. Examples of excipients which can be incorporated in tablets, capsules and the like are the following: a binder such as gum tragacanth, acacia, corn starch or gelatin; an excipient such as microcrystalline cellulose; a disintegrating agent such as corn starch, pregelatinized starch, alginic acid and the like; a lubricant such as magnesium stearate; a sweetening agent such as sucrose, lactose or saccharin; a flavoring agent such as peppermint, oil of wintergreen or cherry. When the dosage unit form is a capsule, it may contain, in addition to materials of the above type, a liquid carrier such as fatty oil. Various other materials may be present as coatings or to otherwise modify the physical form of the dosage unit. For instance, tablets may be coated with shellac, sugar or both. A syrup or elixir may contain the active compound, sucrose as a sweetening agent, methyl and propyl parabens as preservatives, a dye and a flavoring such as cherry or orange flavor. Compositions for intravenous administration may for example be solutions of the compounds of the invention in sterile isotonic aqueous buffer. Where necessary, the intravenous compositions may include for instance solubilizing agents, stabilizing agents and/or a local anesthetic to ease the pain at the site of the injection.

The compounds of the invention may be administered enterally or parenterally. The exact dose and regimen of these compounds and compositions thereof will be dependent on the biological activity of the compound per se, the age, weight and sex of the individual, the needs of the individual subject to whom the medicament is administered, the degree of affliction or need and the judgment of the medical practitioner. In general, parenteral administration requires lower dosages than other methods of administration which are more dependent upon adsorption. However, the dosages for humans are preferably 0.001-10 mg per kg body weight. In general, enteral and parenteral dosages will be in the range of 0.1 to 1,000 mg per day of total active ingredients.

In an embodiment of the invention, a pharmaceutical kit or kit of parts is provided comprising one or more containers filled with one or more pharmaceutical compositions of the invention and optionally one or more pharmaceutically acceptable excipients as described herein. Associated with such container(s) can be various written materials such as instructions for use, or a notice in the form prescribed by a governmental agency regulating the manufacture, use or sale of pharmaceuticals products, which notice reflects approval by the agency of manufacture, use, or sale for human or veterinary administration. Preferably, a pharmaceutical kit or kit of parts comprises instructions for use.

The compounds of the invention are modulators of the S1P receptor, in particular of the S1P5 receptor. More specifically, the compounds of the invention are S1P5 receptor agonists. The compounds are useful in the treatment, alleviation and/or prevention of diseases or disorders mediated by an S1P receptor, preferably S1P5. The compounds of the present invention are particularly suitable to treat, alleviate or prevent diseases and conditions in which an S1P receptor is involved or in which modulation of the endogenous S1P signaling system via an S1P receptor is involved, preferably S1P5.

Provided is therefore a method of treatment, alleviation or prevention of a disease or condition in which an S1P receptor is involved or in which modulation of the endogenous S1P signaling system via an S1P receptor is involved, preferably S1P5, comprising administering to a patient in need thereof a compound according to the invention or a pharmaceutically acceptable salt, solvate, tautomer, stereoisomer or N-oxide thereof. Said patient is preferably a human patient.

Further provided is a use of a compound according to the invention or a pharmaceutically acceptable salt, solvate, tautomer, stereoisomer or N-oxide thereof for the manufacture of a medicament for the treatment, alleviation or prevention of a disease or condition in which an S1P receptor is involved or in which modulation of the endogenous S1P signaling system via an S1P receptor is involved, preferably S1P5 receptor.

Further provided is a compound according to the invention, or a pharmaceutically acceptable salt, solvate, tautomer, stereoisomer or N-oxide thereof for use in therapy, preferably for use as a medicament.

Further provided is a compound according to the invention or a pharmaceutically acceptable salt, solvate, tautomer, stereoisomer or N-oxide thereof, or a pharmaceutical composition comprising such compound, or a pharmaceutically acceptable salt, solvate, tautomer, stereoisomer or N-oxide thereof, for use in the treatment, alleviation or prevention of a disease or condition in which an S1P receptor is involved or in which modulation of the endogenous S1P signaling system via an S1P receptor is involved, preferably S1P5.

Said diseases or condition is preferably selected from the group consisting of Alzheimer's Disease (AD) and associated dementia's, amyloid B-associated disorders, Mild Cognitive Impairment (MCI), Parkinson's Disease (PD), Lewy Body Dementia (LBD), Progressive Supranuclear Palsy (PSP), Cerebral Palsy (CP), Amyotrophic Lateral Sclerosis (ALS), Frontal Temporal Lobe Dementia (FTLD), multiple sclerosis, Huntington's Disease, neurological symptoms of sphingolipidosis disorders, a lysosomal storage disorder including Tay Sachs Disease, Sandhoff Disease, Fabry's Disease, Krabbe Disease, Gaucher's Disease, Niemann Pick A, B or C, and Batten's Disease, stroke, HIV-associated Dementia (HAD), HIV-associate Neurocognitive Disorder (HAND), HIV-associated neuropathy, schizophrenia, cognitive deficits in Schizophrenia, an attention deficit disorder including Anxiety Attention Deficit Disorder and Attention Deficit Hyperactivity Disorder (ADHD), a bipolar disorder, Obsessive-Compulsive Behavior, pain including neuropathic, back pain and pain-associated with multiple sclerosis, spinal cord injury, Parkinson's Disease, epilepsy, diabetes and cancer, cancer-induced peripheral neuropathy (CIPN), depression, treatment-resistant depression, Creutzfeld-Jakob Disease and other Prion-related Disorders, Down's Syndrome, autism, age-related cognitive decline or memory impairment, cognitive deficits associated with diabetes, dementia, dementia associated with Down's Syndrome, cognitive deficits in psychiatric disorders, dementia associated with Lewy Body pathology, diminished CNS function associated with traumatic brain injury, Pick's Disease, spinal cord injury, a demyelinating disorder, a disorder of basal ganglia, AIDS-associated dementia, Psoriasis type 1 and type 2, atopic dermatitis, dermatitis scleroderma, insulin-dependent diabetes mellitus, ulcerative colitis, atherosclerosis, sepsis syndrome, septic shock, Dengue hemorrhagic fever, Dengue, atopic allergy, HIV/AIDS, barrier-integrity associated lung diseases, leukemia, contact dermatitis, encephalomyelitis, Epstein Barr virus infection and other virus infections requiring cell-cell fusion.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described in this document.

Features may be described herein as part of the same or separate aspects or embodiments of the present invention for the purpose of clarity and a concise description. It will be appreciated by the skilled person that the scope of the invention may include embodiments having combinations of all or some of the features described herein as part of the same or separate embodiments.

References described herein are incorporated by reference. Neither these, nor any other documents or citations to any references, are admitted to be prior art documents or citations.

The invention will be explained in more detail in the following, non-limiting examples.

### EXAMPLES

### Abbreviations

AcOH acetic acid
ACN acetonitrile
BOC group tert-butyloxycarbonyl protecting group
DBU diazabicyclo[5.4.0]undec-7-ene
DCM dichloromethane
DIPEA N,N-Diisopropylethylamine
DMA N,N-dimethylacetamide
DMF N,N-dimethylformamide
DMSO dimethyl sulfoxide
EA ethyl acetate
Eq molar equivalent
Et₃N triethylamine
EtOAc ethyl acetate
EtOH ethanol
KOH potassium hydroxyde
KOtBu potassium tert-butoxide
MeOH methanol
MTBE/MTB ether methyl tert-butyl ether
NaOH sodium hydroxide
NMP N-methyl-2-pyrrolidinone
Pd—C palladium-on-carbon
PE petroleum ether
RT room temperature
TLC thin layer chromatography
TFA trifluoroacetic acid
THF tetrahydrofuran
TOTU O-[(ethoxycarbonyl)cyanomethylenamino]-N,N,N′,N′-tetramethyluroniumtetrafluoroborate
v/v volume/volume
XPHOS 2-Dicyclohexylphosphino-2′,4′,6′-triisopropylbiphenyl

### 1. Methods

NMR Spectroscopy and Mass Spectrometry data were obtained by the following methods.

### Nuclear Magnetic Resonance (NMR)

The compounds were either characterized via proton-NMR in d6-dimethylsulfoxide, d-chloroform, d-methanol or d-pyridine on a 400 MHz (Bruker) or 500 MHz NMR instrument (Bruker Avance 500 MHz with 5 mm BBFo-z-Grd) or a 600 MHz (Bruker Avance 600 MHz with 5 mm Cryoprobe CPTCI (1H-13C/15N z-Grd), and/or by mass spectrometry.

The magnetic nuclear resonance spectral properties (NMR) refer to the chemical shifts (δ) expressed in parts per million (ppm). The relative area of the shifts in the 1H-NMR spectrum corresponds to the number of hydrogen atoms for a particular functional type in the molecule. The nature of the shift, as regards multiplicity, is indicated as singlet (s), broad singlet (s. br.), doublet (d), broad doublet (d br.), triplet (t), broad triplet (t br.), quartet (q), quintet (quint.) and multiplet (m).

### Liquid Chromatography-Mass Spectrometry (LC-MS)

Analytical samples were run on Agilent 1200 series instruments controlled by Agilent ChemStation Software. The system consists of an injector, a column compartment for 2 columns, a binary solvent pump, an UV-detector and a quadrupole mass spectrometer (Agilent 6100 series, ESI-ionization). If not stated otherwise, the mobile phase consisted of water and acetonitrile, both acidified with 0.1% formic acid. Separation was performed on YMC Meteoric Core C18 columns with 50 mm in length, 2.1 mm in diameter and packed with 2.7 μm material. Elution was done at 50° C. with a linear gradient ramping from 5 to 100% organic solvent over 1.8 min at a constant flow rate of 1 mL/min.

### High-Performance Liquid Chromatography (HPLC)

For compounds 12 to compound 74 the crude products were purified by preparative HPLC using one of the TFA or AA method described below.

HPLC was run on The HPLC instrument used was an Agilent 1100 Series. The columns used were Waters Sunfire C8 5 μM 150×30 mm or Phenomenex Luna AXIA column.

### Trifluoro Acetic Acid Method (TFA Method):

A gradient of acetonitrile (A) and 0.1% trifluoroacetic acid in water (B) was used, at a flow rate of 50 mL/min (0-0.5 min 5% A, 0.5-8.5 min linear gradient 05-100% A, 8.7-10.7 min 100% A, 10.7-11 min linear gradient 100-05% A).

### Ammonium Acetate Method (AA Method):

A gradient of acetonitrile (A) and 10 mM ammonium acetate in water (B) was used, at a flow rate of 50 mL/min (0-0.5 min 5% A, 0.5-8.5 min linear gradient 05-100% A, 8.7-10.7 min 100% A, 10.7-11 min linear gradient 100-05% A).

Samples were injected in 1.5 mL DMSO:MeOH (1:1). An Agilent 1100 Series Purification system was used, consisting of the following modules: Agilent 1100 Series LC/MSD SL mass spectrometer with API-electrospray source; two Agilent 1100 Series preparative pumps; Agilent 1100 Series isocratic pump; Agilent 1100 Series diode array detector with preparative (0.3 mm) flow cell; Agilent active-splitter, IFC-PAL fraction collector/autosampler. The make-up pump for the mass spectrometer used 3:1 methanol:water with 0.1% formic acid at a flow rate of 1 mL/min. Fraction collection was automatically triggered when the extracted ion chromatogram (EIC) for the target mass exceeded the threshold specified in the method. The system was controlled using Agilent Chemstation (Rev B.10.03), Agilent A2Prep, and Leap FractPal software, with custom Chemstation macros for data export.

### 2. General Synthesis Methods

Suitable methods to synthesize compounds of the invention are described below.

### Synthesis of Compound X

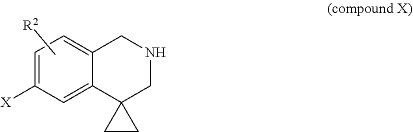

Different synthesis routes to prepare R2-Substituted 2′,3′-dihydro-1′H-spiro[cyclopropane-1,4′-isoquinoline] (intermediate compound X) are known in the art. Three possible synthesis routes are depicted in Scheme 1.

Synthesis route a) in Scheme 1 shows a synthesis route analogue to procedures described in US20120225857. Starting with R2-substituted 6-bromo-2,3-dihydro-1H-inden-1-ones, followed by reduction of the corresponding 2′,3′-dihydro-1′H-spiro[cyclopropane-1,4′-isoquinolin]-1′-ones (e.g. with borane tetrahydrofuran complex) could give the corresponding R2-substituted 2′,3′-dihydro-1′H-spiro[cyclopropane-1,4′-isoquinolines].

Synthesis route b) in Scheme 1 shows alkylation of R2-substituted alkyl 2-(cyanomethyl)benzoate with e.g. 1-bromo-2-chloroethane in the presence of base (e.g. NaH, c.f. WO2016100161), followed by reduction of the cyano moiety (e.g. using cobalt (II) chloride and sodium borohydride, c.f. US20150361067) and subsequent reduction of the corresponding 2′,3′-dihydro-1′H-spiro[cyclopropane-1,4′-isoquinolin]-1′-one (e.g. with borane tetrahydrofuran complex).

Synthesis route c) in Scheme 1 shows alkylation of R2-substituted 2-phenylacetonitriles with e.g. 1-bromo-2-chloroethane in the presence of base (e.g. NaH, c.f. WO2016100161), followed by reduction of the cyano moiety (e.g. using cobalt (II) chloride and sodium borohydride, c.f. US20150361067 or lithium aluminium hydride). Reaction of the primary amine with methyl carbonochloridate in the presence of base (e.g. triethyl amine) followed by cyclization using polyphospheric acid and elevated temperatures (c.f. WO2013079452) could give the corresponding R-2 substituted 2′,3′-dihydro-1′H-spiro[cyclopropane-1,4′-isoquinolin]-1′-ones which could be reduced (e.g. with borane tetrahydrofuran complex) to the corresponding R2-substituted 2′,3′-dihydro-1′H-spiro[cyclopropane-1,4′-isoquinolines].

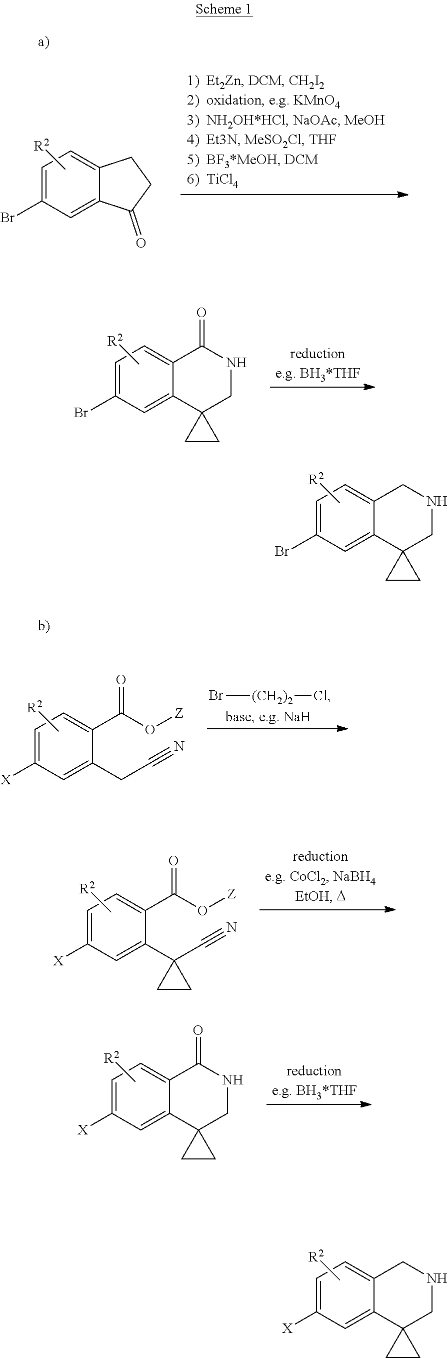

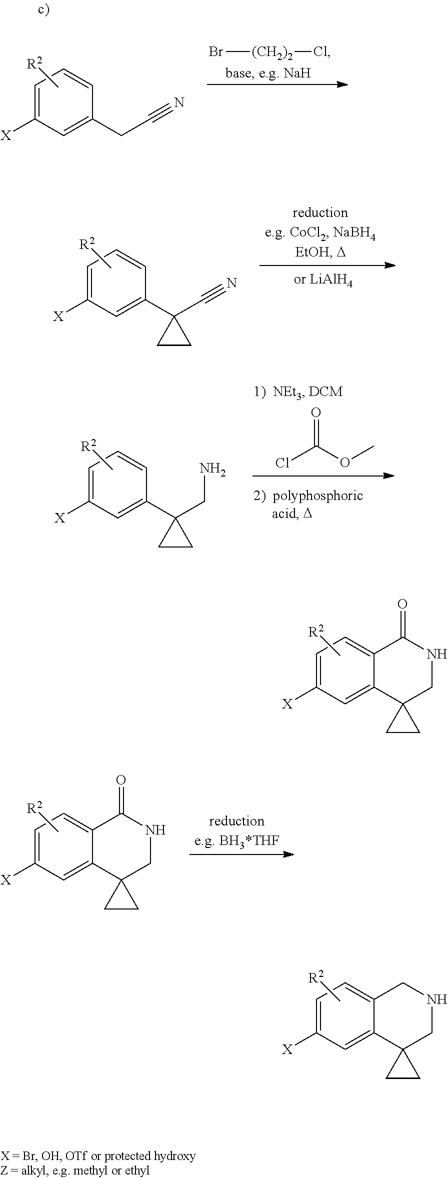

### Introduction R1 Group (Intermediate Compounds I-II and IV-V and VII-IX)

Synthetic routes for the introduction of the R1 group are shown in scheme 2. R1-moieties depicted in scheme 2 above can be introduced starting from the corresponding R2-substituted tetrahydroisoquinolines (e.g. 2′,3′-dihydro-1′H-spiro[cyclopropane-1,4′-isoquinolin]-6′-ol, 6′-bromo-2′,3′-dihydro-1′H-spiro[cyclopropane-1,4′-isoquinoline] or 2′,3′-dihydro-1′H-spiro[cyclopropane-1,4′-isoquinolin]-6′-yl trifluoromethanesulfonate) using alkylating or reductive amination conditions. When the R1-moiety contains an ester the corresponding acid can be obtained by saponification under basic (e.g. NaOH) or acidic (e.g. TFA) conditions. Scheme 2 shows a number of routes starting from R2-substituted 6′-bromo-2′,3′-dihydro-1′H-spiro[cyclopropane-1,4′-isoquinoline]. It is clear to a person skilled in the art that these routes are suitable to introduce alternative substituents in these tetrahydroisoquinolines using the appropriate reagent(s).

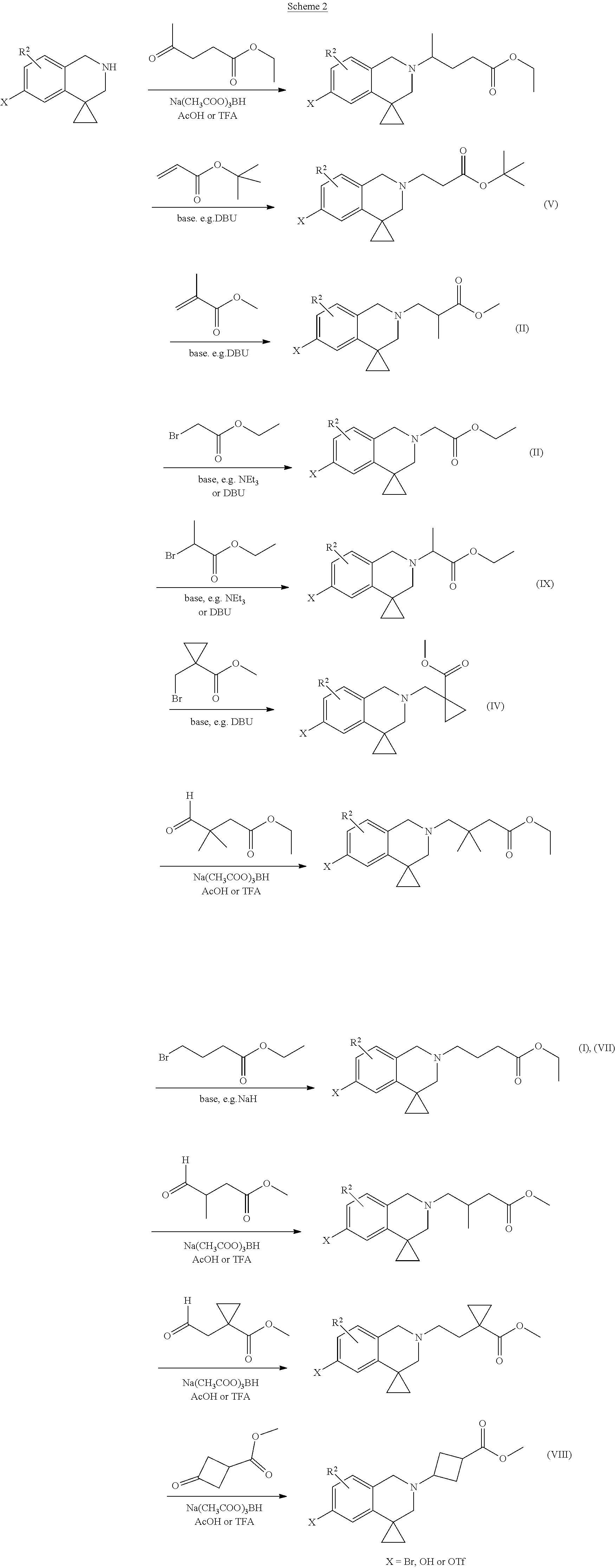

### Trifluoromethanesulfonate Intermediates (Intermediate Compound III)

R1-substituted and R2-substituted 2′,3′-dihydro-1′H-spiro[cyclopropane-1,4′-isoquinolin]-6′-ols can be converted to the corresponding R1-substituted and R2-substituted 2′,3′-dihydro-1′H-spiro[cyclopropane-1,4′-isoquinolin]-6′-yl trifluoromethanesulfonates using trifluoro acetic acid and a base (e.g. pyridine). Analogously R2-substituted 2′,3′-dihydro-1′H-spiro[cyclopropane-1,4′-isoquinolin]-6′-yl trifluoromethanesulfonates can be prepared starting from N-protected (e.g. BOC protected) R2-substituted 2′,3′-dihydro-1′H-spiro[cyclopropane-1,4′-isoquinolin]-6′-ols using using trifluoro acetic acid and a base (e.g. pyridine). Deprotection (e.g. treatment with acid like TFA or HCl when the protective group is acid labile e.g. BOC) can give the corresponding R2-substituted 2′,3′-dihydro-1′H-spiro[cyclopropane-1,4′-isoquinolin]-6′-yl trifluoromethanesulfonates.

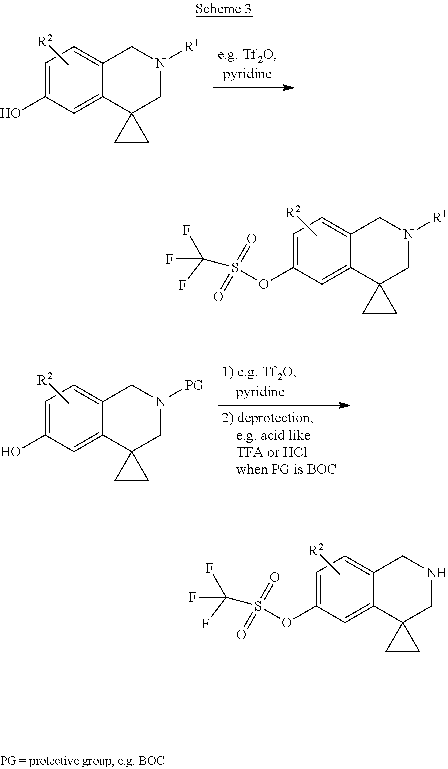

### Introduction of the R3-L1 Group

Synthetic routes for introducing the R3-L1 group are shown in Scheme 4. R3-L1-moieties can be introduced starting from the corresponding R1-substituted and R2-substituted 6′-bromo-2′,3′-dihydro-1′H-spiro[cyclopropane-1,4′-isoquinoline] or R1-substituted and R2-substituted 2′,3′-dihydro-1′H-spiro[cyclopropane-1,4′-isoquinolin]-6′-yl trifluoromethanesulfonate by Sonogashira reaction with an substituted alkyne or by the same protocol followed by hydrogenation which can yield the corresponding ethenyl derivative as shown in scheme 4. R3-L1-moieties that contain a phenyl ether can be obtained under alkylating conditions starting from the corresponding R1-substituted and R2-substituted 2′,3′-dihydro-1′H-spiro[cyclopropane-1,4′-isoquinolin]-6′-ol using the corresponding alkylhalide precursors or under Mitsunobu conditions using the corresponding alkyl alcohols.

When the R1-moiety contains an ester the corresponding acid can be obtained by saponification under basic (e.g. NaOH) or acidic (e.g. TFA) conditions.

In the following section the synthesis of compounds according to the invention below, the introduction of alternative linkers L1 and R3-L1-moieties is described. It is clear to a skilled person that the routes described for specific compounds can be used to introduce the same linker L1 and R3-L1-moieties in other compounds according to the invention.

Fluorinated intermediates required to prepare compounds with fluorinated R1-moieties can be prepared using methods described in the literature (e.g.: a) Pier Alexandre Champagne, Justine Desroches, Jean-Denys Hamel, Mathilde Vandamme, and Jean-François PaquinChem. Rev. 2015, 115, 9073-9174; b) Jimmy Wu, Tetrahedron Letters 55 (2014) 4289-4294 and references cited therein).

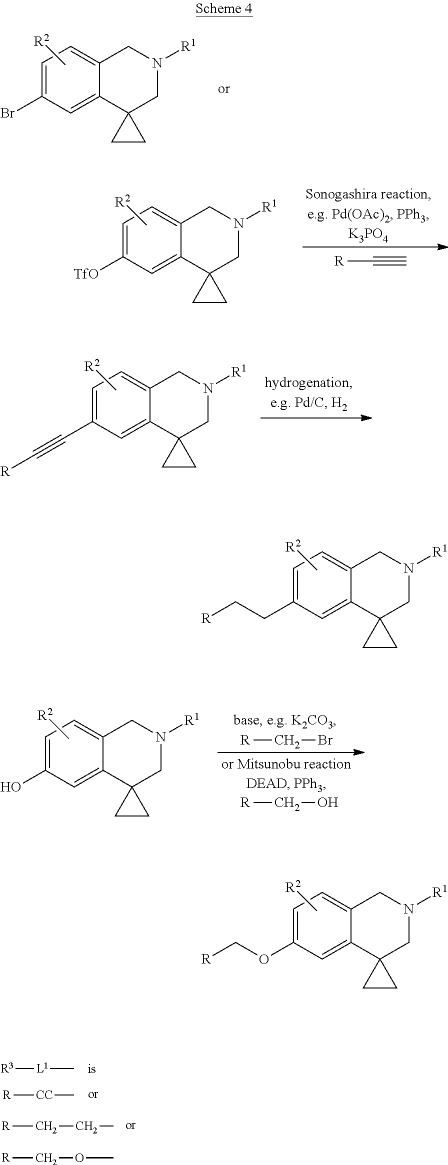

### 3. Synthesis of Intermediates

### General Method for Synthesis of Intermediate Compound I

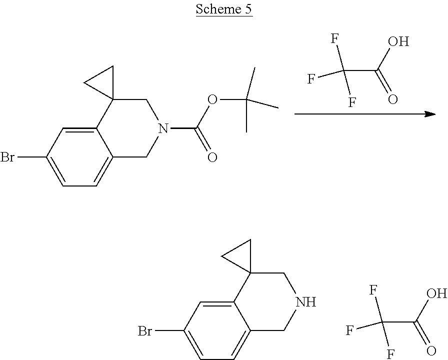

Intermediate compound X was first obtained by removing the protective group from 6′-Bromo-2′,3′-dihydro-1′H-spiro[cyclopropane-1,4′-isoquinoline] with 2,2,2-trifluoroacetate. In a 100 mL round-bottomed flask was tert-butyl 6′-bromo-2′,3′-dihydro-1′H-spiro[cyclopropane-1,4′-isoquinoline]-2′-carboxylate (2 g, 5.91 mmol) was dissolved in dichloromethane (25 mL) to give a colorless solution.

Trifluoroacetic acid (5 mL, 64.9 mmol) was added. The reaction mixture was stirred at room temperature overnight. The reaction mixture was evaporated. The residue was treated with 20 mL methyl-tert-butylether at room temperature. A white solid appears. The slurry was stirred at room temperature for 30 min, filtered and dried at 40° C. under vacuum.

Yield: 1.9 g (5.40 mmol, 91%, white solid)

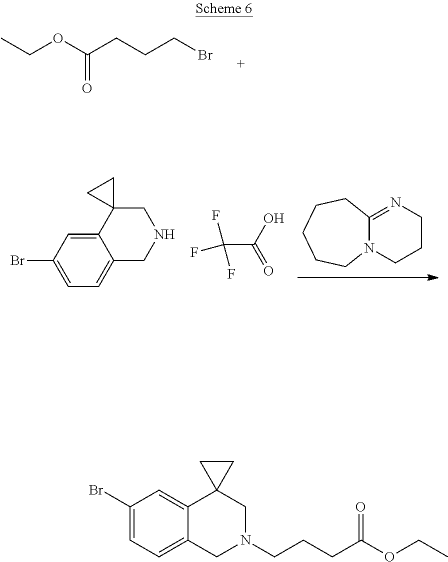

Ethyl 4-(6′-bromo-1′H-spiro[cyclopropane-1,4′-isoquinolin]-2′(3′H)-yl)butanoate (intermediate compound I) was prepared as follows.

In a 50 mL round-bottomed flask was 6′-bromo-2′,3′-dihydro-1′H-spiro[cyclopropane-1,4′-isoquinoline] 2,2,2-trifluoroacetate (850 mg, 2.414 mmol) was dissolved in DMF (15 mL) to give a colorless solution. DBU (1.0 mL, 6.63 mmol) and ethyl 4-bromobutyrate (1 mL, 6.99 mmol) was added. The reaction mixture was stirred at room temperature overnight. Additional DBU (0.5 mL, 3.32 mmol) and ethyl 4-bromobutyrate (0.5 mL, 3.5 mmol) were added and the mixture was stirred at room temperature for further 2 days.

The solvent was evaporated, the residue was dissolved in dichloromethane and washed with sat. NH₄Cl-solution. After phase separation with a Chromabond-PTS cartridge the organic layer was evaporated. The residue was purified by flash-chromatography (40 g, 0-5% methanol in dichloromethane).

Yield: 488 mg (2.058 mmol, 85%, yellow oil)

### General Method for Synthesis of Intermediate Compound II

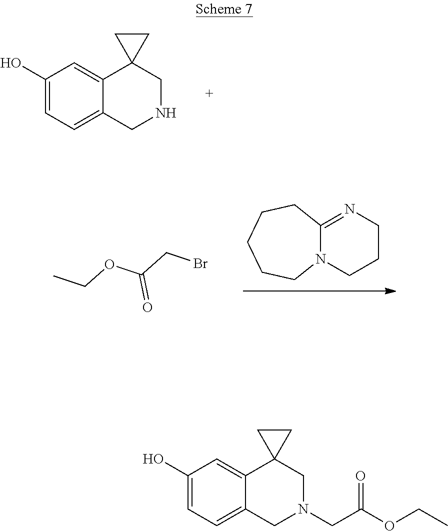

Ethyl 2-(6′-hydroxy-1′H-spiro[cyclopropane-1,4′-isoquinolin]-2′(3′H)-yl)acetate (intermediate compound II) was prepared as follows.

In a 100 mL round-bottomed flask 2′,3′-dihydro-1′H-spiro[cyclopropane-1,4′-isoquinolin]-6′-ol (2 g, 11.41 mmol) was dissolved in DMF (50 mL) to give a yellow solution. DBU (2.5 mL, 16.59 mmol) and ethyl bromoacetate (1.5 mL, 13.53 mmol) were added (exothermic reaction, the temperature rose to 30° C.). The reaction mixtures was stirred at room temperature overnight.

The solvent was evaporated, the residue was dissolved in dichloromethane and washed twice with sat. NH₄Cl-solution and washed once with saturated sodium chloride solution. The organic layer was dried over MgSO₄, filtered and evaporated. The residue was purified by flash-chromatography (40 g, 0-10% methanol in dichloromethane). The product was dried under vacuum at 40° C.

Yield: 1.53 g (white solid)

### General Method for Synthesis of Intermediate Compound III

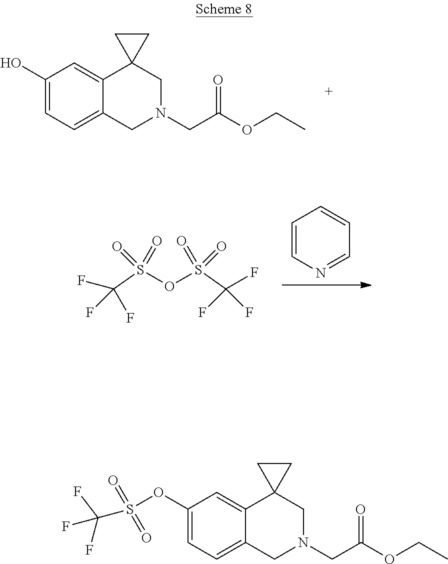

Ethyl 2-(6′-(((trifluoromethyl)sulfonyl)oxy)-1′H-spiro[cyclopropane-1,4′-isoquinolin]-2′(3′H)-yl)acetate (intermediate compound III) was prepared as follows.

In a 100 mL 3-neck round-bottomed flask ethyl 2-(6′-hydroxy-1′H-spiro[cyclopropane-1,4′-isoquinolin]-2′(3′H)-yl)acetate (500 mg, 1.913 mmol) was suspended in dichloromethane (15 mL). Pyridine (0.4 mL, 4.95 mmol) was added. The mixture was cooled to 0° C. and at this temperature trifluoromethaneslufonic acid anhydride (2 mL, 2.000 mmol) was added dropwise. The reaction mixture was stirred at room temperature overnight.

The reaction mixture was diluted with dichloromethane, washed twice with sat. NH₄Cl-solution and washed once with saturated sodium chloride solution. The organic layer was dried over MgSO₄, filtered and evaporated. The residue was purified by flash-chromatography (12 g, 0-10% methanol in dichloromethane).

Yield: 547 mg (yellow oil)

### General Method for Synthesis of Intermediate Compound IV

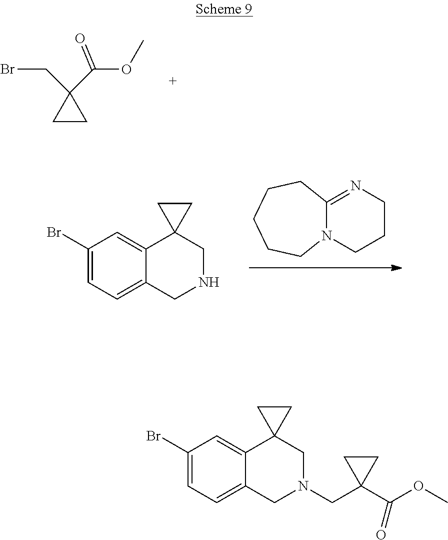

Methyl 1-((6′-bromo-1′H-spiro[cyclopropane-1,4′-isoquinolin]-2′(3′H)-yl)methyl)cyclopropanecarboxylate (intermediate compound IV) was prepared according to scheme 9.

In a 50 mL round-bottomed flask 6′-bromo-2′,3′-dihydro-1′H-spiro[cyclopropane-1,4′-isoquinoline] (1 g, 4.20 mmol) was treated with DMF (10 mL) to give a white suspension. DBU (1.5 mL, 9.95 mmol) was added (clear solution) and after 5 min methyl 1-(bromomethyl)cyclopropanecarboxylate (1 g, 5.18 mmol) was added. The reaction mixture was stirred at room temperature overnight.

The solvent was evaporated, the residue was dissolved in dichloromethane, washed twice with sat. NH₄Cl-solution, dried over MgSO₄, filtered and evaporated. The residue was purified by flash-chromatography (24 g, 0-10% methanol in dichloromethane, 35 mL/min)

Yield: 800 mg (orange oil)

### General Method for Synthesis of Intermediate Compound V

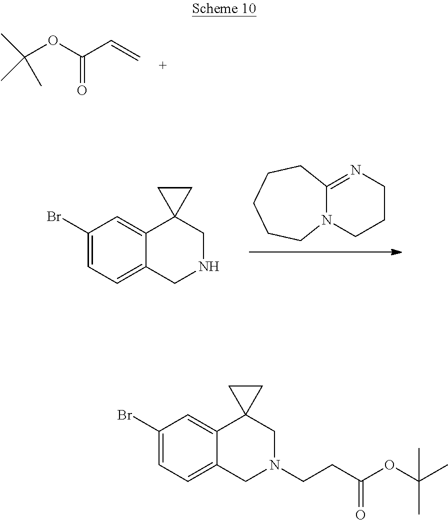

Tert-butyl 3-(6′-bromo-1′H-spiro[cyclopropane-1,4′-isoquinolin]-2′(3′H)-yl)propanoate (intermediate compound V) was prepared according to scheme 10. 6′-bromo-2′,3′-dihydro-1′H-spiro[cyclopropane-1,4′-isoquinoline] (1.9 g, 7.98 mmol) was dissolved in methanol (100 mL) and DBU (0.972 g, 6.38 mmol) and tert.butylacrylate (3.07 g, 23.94 mmol) were added under stirring at room temperature. Stirring was continued for 4 h. The solvent was evaporated, the residue was dissolved in dichloromethane, washed sequentially with 10% aqueous ammonium chloride solution and water. The solution of the crude product was dried (MgSO₄), filtered and the solvent was evaporated. The crude product was purified by flash chromatography (dichloromethane/methanol 98:2).

Yield: 2.4 g (6.55 mmol, 82%, light yellow oil).

### General Method for Synthesis of Intermediate Compound VI

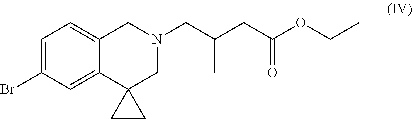 [SMILES: CCOC(=O)CC(C)CN1Cc2ccc(Br)cc2C2(CC2)C1]

Ethyl 4-(6′-bromo-1′H-spiro[cyclopropane-1,4′-isoquinolin]-2′(3′H)-yl)-3-methylbutanoate (intermediate compound VI) was prepared according to scheme 3.

In a 250 mL round-bottomed flask 6′-bromo-2′,3′-dihydro-1′H-spiro[cyclopropane-1,4′-isoquinoline] 2,2,2-trifluoroacetate (500 mg, 1.420 mmol) was dissolved in tetrahydrofuran (100 mL) and ethyl 3-methyl-4-oxobutanoate (250 mg, 1.734 mmol) and triethylamine (0.178 mL, 1.278 mmol) were added to give a colorless solution. The reaction mixture was stirred for 3 h at room temperature. sodium triacetoxyborohydride (451 mg, 2.130 mmol) was added in small portions. The reaction mixture was stirred at room temperature overnight.

The reaction mixture was diluted with dichloromethane and water. The reaction mixture was stirred for 10 min at room temperature. After phase separation the organic layer was washed with saturated sodium chlorid solution, dried (MgSO₄), filtered and evaporated. The residue was purified by flash chromatography (silica 12 g, 0-15% methanol in dichloromethane).

Yield: 430 mg (1.174 mmol, 83%, orange oil).

### General Method for Synthesis of Intermediate Compound VII

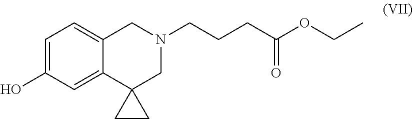 [SMILES: CCOC(=O)CCCN1Cc2ccc(O)cc2C2(CC2)C1]

Ethyl 4-(6′-hydroxy-1′H-spiro[cyclopropane-1,4′-isoquinolin]-2′(3′H)-yl)butanoate (compound VII) was prepared as follows.

In a 100 mL round-bottomed flask 2′,3′-dihydro-1′H-spiro[cyclopropane-1,4′-isoquinolin]-6′-ol (2 g, 11.41 mmol) was dissolved in DMF (30 mL) to give a colorless solution. DBU (2.5 mL, 16.59 mmol) and ethyl 4-bromobutyrate (2 mL, 13.98 mmol) were added. The reaction mixtures was stirred at room temperature overnight.

The reaction mixture was evaporated, the residue was dissolved in dichloromethane and washed twice with saturated ammonium chloride solution and once with saturated sodium chloride solution. The organic layer was dried (MgSO₄), filtered and evaporated. The residue was purified flash chromatography (silica 40 g, 0-10% methanol in dichloromethane).

Yield: 2.18 g (7.53 mmol, 66%, yellow solid).

### General Method for Synthesis of Intermediate Compound VIII

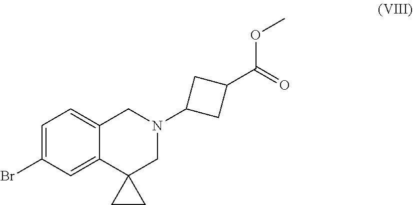 [SMILES: COC(=O)C1CC(N2Cc3ccc(Br)cc3C3(CC3)C2)C1]

Methyl 3-(6′-bromo-1′H-spiro[cyclopropane-1,4′-isoquinolin]-2′(3′H)-yl)cyclobutane-1-carboxylate was prepared as follows.

In a 50 mL round-bottomed flask 6′-bromo-2′,3′-dihydro-1′H-spiro[cyclopropane-1,4′-isoquinoline] (1 g, 4.20 mmol) was dissolved in tetrahydrofuran (25 mL) and methyl 3-oxocyclobutanecarboxylate (0.538 g, 4.20 mmol) was added to give a colorless solution. The reaction mixture was stirred at room temperature for 1 h. sodium triacetoxyborohydride (1.8 g, 8.49 mmol) was added. The reaction mixture was stirred at room temperature overnight.

The reaction mixture was diluted with dichloromethane and water. After phase separation with a the organic layer was evaporated. The residue was purified by flash chromatography (silica 12 g, 0-20% methanol in dichloromethane). The oil was dissolved in ethylacetate and washed twice with saturated sodium bicarbonate solution, once with saturated sodium chloride solution, dried over (MgSO₄), filtered and evaporated.

Yield: 1.15 g (3.28 mmol, 78%. orange oil).

### General Method for Synthesis of Intermediate Compound IX

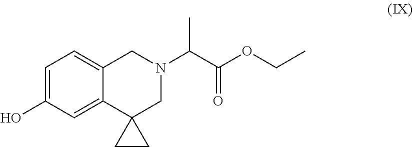 [SMILES: CCOC(=O)C(C)N1Cc2ccc(O)cc2C2(CC2)C1]

Ethyl 2-(6′-hydroxy-1′H-spiro[cyclopropane-1,4′-isoquinolin]-2′(3′H)-yl)propanoate (compound IX) was prepared as follows.

In a 100 mL round-bottomed flask 2′,3′-dihydro-1′H-spiro[cyclopropane-1,4′-isoquinolin]-6′-ol (2 g, 11.41 mmol) was dissolved in DMF (30 mL) to give a colorless solution. DBU (2.5 mL, 16159 mmol) and ethyl 2-bromopropionate (2 mL, 15.40 mmol) were added. The reaction mixture was stirred at room temperature overnight.

The reaction mixture was evaporated, the residue was dissolved in dichloromethane, washed twice with saturated ammonium chloride solution and once with saturated sodium chloride solution. The organic layer was dried (MgSO₄), filtered and evaporated. The residue was purified by flash chromatography (silica 40 g, 0-10% methanol in dichloromethane).

Yield: 1.68 g (6.1 mmol, 54%, yellow oil).

### 4. Synthesis of Compounds According to the Invention

### 2′-(3-carboxypropyl)-6′-((4-isopropylphenyl)ethynyl)-2′,3′-dihydro-1′H-spiro[cyclopropane-1,4′-isoquinolin]-2′-ium 2,2,2-trifluoroacetate (Compound 1)

Ethyl 4-(6′-((4-isopropylphenyl)ethynyl)-1′H-spiro[cyclopropane-1,4′-isoquinolin]-2′(3′H)-yl)butanoate (compound 1) was prepared by reacting Intermediate compound I with 4′-isopropylphenyl acetylene according to scheme 11.

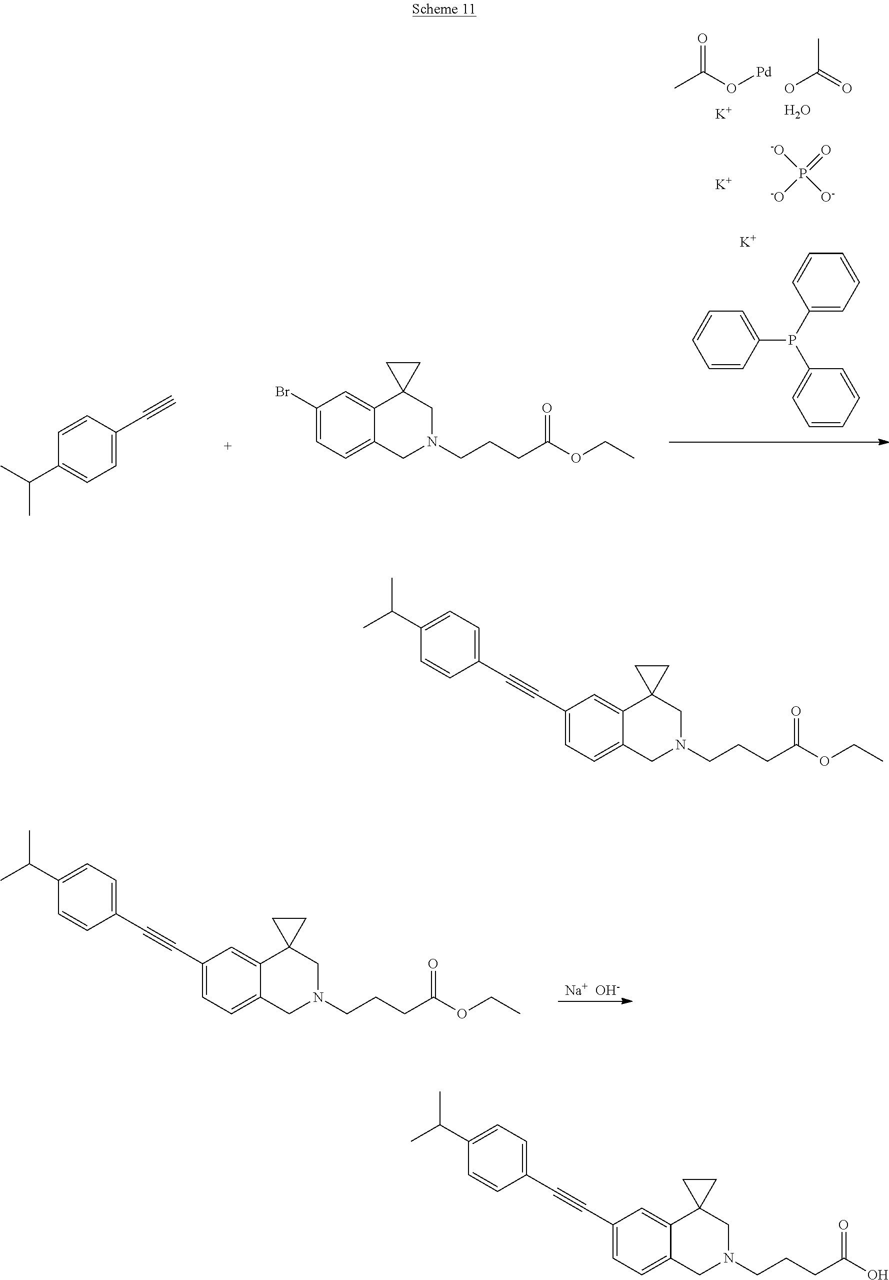

In a Schlenck flask were triphenylphosphine (14.89 mg, 0.057 mmol), palladium(II) acetate (3.82 mg, 0.017 mmol) and potassium phosphate tribasic monohydrate (78 mg, 0.341 mmol) was dried for 30 min under Argon. In a second flask were 4′-isopropylphenyl acetylene (49.1 mg, 0.341 mmol) and ethyl 4-(6′-bromo-1′H-spiro[cyclopropane-1,4′-isoquinolin]-2′(3′H)-yl)butanoate (100 mg, 0.284 mmol) was dissolved in DMSO (2 mL). This solution was transferred into the first Schlenck flask via syringe and heated to 80° C. for 1 h.

The reaction mixture was diluted with dichloromethane and washed twice with water and once with saturated sodium chloride solution. The organic layer was passed through a Chromabond PTS-cartridge and evaporated. The residue was purified by flash-chromatography (12 g, 0-10% methanol in dichloromethane) Yield: 103 mg (0.248 mmol, 87%, brown oil, contains a rest of DMSO).

In a 50 mL round-bottomed flask was ethyl 4-(6′-((4-isopropylphenyl)ethynyl)-1′H-spiro[cyclopropane-1,4′-isoquinolin]-2′(3′H)-yl)butanoate (103 mg, 0.248 mmol) was dissolved in mixture of tetrahydrofuran (2 mL) and methanol (2 mL) to give a colorless solution. 1M aqueous NaOH (0.5 mL, 1.000 mmol) was added. The reaction mixture was stirred at room temperature overnight.

The reaction mixture was evaporated to yield a brown oil (contains DMSO and salts).

The crude product was purified by preparative HPLC (WATERS XBRIDGE C18 OBD, acetonitrile, water, 0.1% trifluoro acetic acid). The purified product was stirred in methyl-tert-butyl ether (1 mL) until it formed a precipitate. The product was filtered and dried overnight under vacuum Yield: 41 mg (0.106 mmol, 43%, white solid).

Calculated mass (C₂₆H₂₉NO₂) 387.2; found mass: 388 (M+H⁺).

¹H NMR (600 MHz, DMSO-d₆) δ 12.37 (s, 1H), 10.07 (s, 1H), 7.49-7.44 (m, 2H), 7.39 (dd, J=7.9, 1.6 Hz, 1H), 7.33-7.29 (m, 2H), 7.26 (d, J=8.1 Hz, 1H), 7.04 (s, 1H), 4.67 (s, 1H), 4.48 (s, 1H), 3.56 (s, 1H), 3.21 (s, 3H), 2.92 (p, J=6.9 Hz, 1H), 2.37 (d, J=7.2 Hz, 2H), 1.96 (s, 2H), 1.46 (s, 1H), 1.21 (d, J=6.9 Hz, 7H). 28 protons.

The following compounds were prepared in the same way as compound 1.

### 4-(6′-((6-isopropoxypyridin-3-yl)ethynyl)-1′H-spiro[cyclopropane-1,4′-isoquinolin]-2′(3′H)-yl)butanoic acid (Compound 2)

Calculated mass (C₂₅H₂₈N₂O₃): 404.2; found mass: 405 (M+H⁺).

¹H NMR (600 MHz, Methanol-d4) δ 8.30-8.25 (m, 1H), 7.74 (dd, J=8.6, 2.4 Hz, 1H), 7.36 (dd, J=8.0, 1.6 Hz, 1H), 7.22 (d, J=8.0 Hz, 1H), 7.02 (d, J=1.6 Hz, 1H), 6.73 (dd, J=8.6, 0.8 Hz, 1H), 5.28 (hept, J=6.2 Hz, 1H), 4.54 (s, 2H), 3.40 (s, 2H), 3.28 (d, J=7.4 Hz, 2H), 2.50 (t, J=6.6 Hz, 2H), 2.06 (p, J=6.9 Hz, 2H), 1.33 (d, J=6.2 Hz, 6H), 1.28 (s, 2H), 1.24-1.18 (m, 2H). 27 protons.

### 2′-(3-carboxypropyl)-6′-((4-ethoxyphenyl)ethynyl)-2′,3′-dihydro-1′H-spiro[cyclopropane-1,4′-isoquinolin]-2′-ium 2,2,2-trifluoroacetate (Compound 3)

Calculated mass (C₂₅H₂₇NO₃): 389.2; found mass: 390 (M+H⁺).

¹H NMR (600 MHz, DMSO-d6) δ 12.37 (s, 1H), 10.08 (s, 1H), 7.49-7.44 (m, 2H), 7.37 (dd, J=7.9, 1.6 Hz, 1H), 7.25 (d, J=8.0 Hz, 1H), 7.02 (d, J=1.9 Hz, 1H), 7.00-6.94 (m, 2H), 4.56 (d, J=108.1 Hz, 3H), 4.06 (d, J=7.0 Hz, 2H), 3.54 (s, 1H), 3.20 (s, 3H), 2.37 (d, J=7.2 Hz, 2H), 1.95 (p, J=7.5 Hz, 2H), 1.44 (t, J=7.0 Hz, 0H), 1.33 (t, J=7.0 Hz, 3H), 1.11 (s, 1H). 26 protons.

### 4-(6′-((1-(cyclopropylmethyl)-6-oxo-1,6-dihydropyridin-3-yl)ethynyl)-1′H-spiro[cyclopropane-1,4′-isoquinolin]-2′(3′H)-yl)butanoic acid (Compound 4)

Calculated mass (C₂₆H₂₈N₂O₃): 416.2; found mass: 417 (M+H⁺).

¹H NMR (500 MHz, DMSO-d₆) δ 8.15 (d, J=2.5 Hz, 1H), 7.68-7.44 (m, 1H), 7.30-7.01 (m, 2H), 6.82 (d, J=1.6 Hz, 1H), 6.43 (d, J=9.5 Hz, 1H), 3.88-3.66 (m, 4H), 1.73 (p, J=7.1 Hz, 2H), 1.05-0.79 (m, 4H), 0.59-0.28 (m, 4H). 20 protons.

### 2′-(carboxymethyl)-6′-((4-ethoxyphenyl)ethynyl)-2′,3′-dihydro-1′H-spiro[cyclopropane-1,4′-isoquinolin]-2′-ium 2,2,2-trifluoroacetate (Compound 5)

Compound 5 was prepared from Intermediate compound III according to reaction scheme 12.

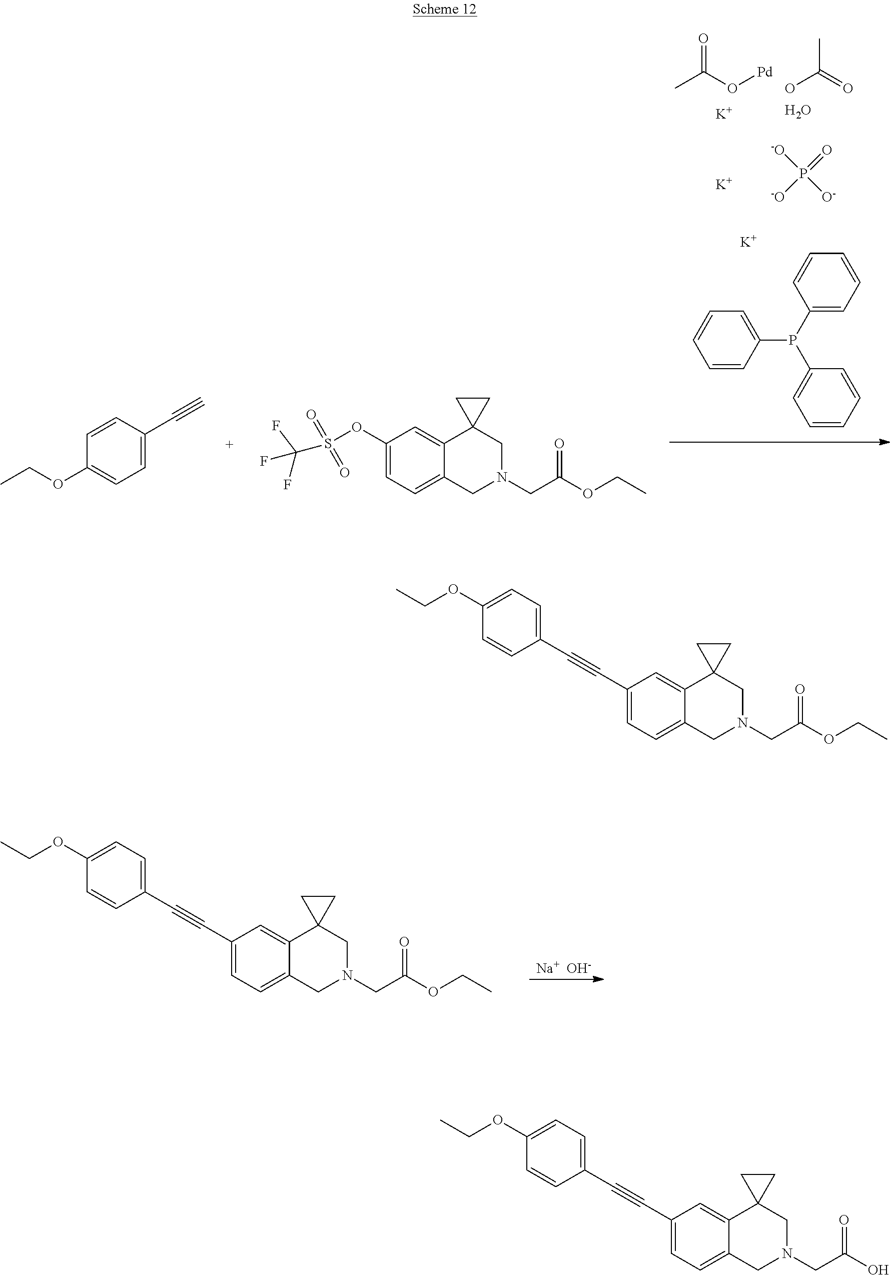

In a Schlenck flask triphenylphosphine (13.34 mg, 0.051 mmol), palladium(II) acetate (3.42 mg, 0.015 mmol) and potassium phosphate tribasic monohydrate (70.2 mg, 0.305 mmol) were dried for 30 min under Argon. In a second flask 4-ethoxyphenylacetylene (55.7 mg, 0.381 mmol) and ethyl 2-(6′-(((trifluoromethyl)sulfonyl)oxy)-1′H-spiro[cyclopropane-1,4′-isoquinolin]-2′(3′H)-yl)acetate (100 mg, 0.254 mmol) were dissolved in DMSO (2 mL) under Argon. This solution was transferred into the first Schlenck flask via syringe and heated to 80° C. for 1 h.

The reaction mixture was diluted with dichloromethane and washed twice with water and once with saturated sodium chloride solution. The organic layer was separated and evaporated. The residue was purified by flash-chromatography (12 g, 0-10% methanol in dichloromethane)

Yield: 84 mg brown oil (contains a rest of DMSO)

In a 50 mL round-bottomed flask ethyl 2-(6′-((4-ethoxyphenyl)ethynyl)-1′H-spiro[cyclopropane-1,4′-isoquinolin]-2′(3′H)-yl)acetate (84 mg, 0.216 mmol) was dissolved in mixture of tetrahydrofuran (2 mL) and methanol (2 mL) to give a colorless solution. 2M aqueous NaOH solution (0.5 mL, 1.000 mmol) was added. The reaction mixture was stirred at room temperature overnight. The reaction mixture was evaporated. The crude product was purified by preparative HPLC (WATERS XBRIDGE C18 OBD, acetonitrile water, 0.1% trifluoro acetic acid). Yield: 30 mg (0.083 mmol, 39%, yellow foam)

Calculated mass (C₂₃H₂₃NO₃): 361.2; found mass: 362 (M+H⁺).

¹H NMR (500 MHz, DMSO-d₆) δ 7.51-7.42 (m, 2H), 7.34 (dd, J=8.0, 1.6 Hz, 1H), 7.23 (d, J=8.0 Hz, 1H), 7.03-6.93 (m, 3H), 4.51 (s, 2H), 4.08 (dd, J=13.4, 6.4 Hz, 4H), 3.34 (s, 2H), 1.33 (t, J=6.9 Hz, 3H), 1.29-1.03 (m, 5H). 23 protons.

The following compounds were prepared in the same way as compound 5.

### 2′-(carboxymethyl)-6′-((4-isopropylphenyl)ethynyl)-2′,3′-dihydro-1′H-spiro[cyclopropane-1,4′-isoquinolin]-2′-ium 2,2,2-trifluoroacetate (compound 6)

Calculated mass (C₂₄H₂₅NO₂): 359.2; found mass: 360 (M+H⁺).

¹H NMR (600 MHz, DMSO-d₆) δ 7.50-7.42 (m, 2H), 7.35 (dd, J=7.9, 1.6 Hz, 1H), 7.33-7.28 (m, 2H), 7.22 (d, J=8.0 Hz, 1H), 7.01 (d, J=1.6 Hz, 1H), 4.44 (s, 2H), 4.00 (s, 2H), 3.26 (s, 2H), 2.92 (p, J=6.8 Hz, 1H), 1.21 (d, J=6.9 Hz, 9H), 1.08 (s, 2H). 25 protons.

### 1-((6′-((4-isopropoxyphenyl)ethynyl)-1′H-spiro[cyclopropane-1,4′-isoquinolin]-2′(3′H)-yl)methyl)cyclopropanecarboxylic acid (Compound 8)

Compound 8 was prepared from intermediate compound IV according to reaction scheme 13.

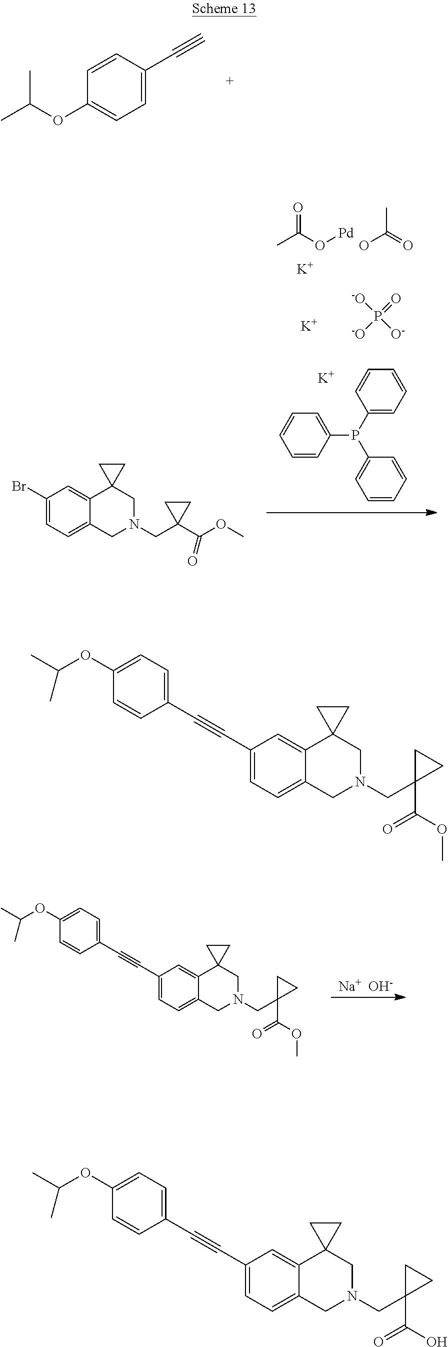

In a 25 mL 3-neck round-bottom flask was palladium(II) acetate (5 mg, 0.022 mmol), triphenylphosphine (20 mg, 0.076 mmol) and potassium phosphate tribasic (45 mg, 0.212 mmol) were stirred under argon atmosphere for 30 min. In a second flask was methyl 1-((6′-bromo-1′H-spiro[cyclopropane-1,4′-isoquinolin]-2′(3′H)-yl)methyl)cyclopropanecarboxylate (60 mg, 0.171 mmol) and 1-ethynyl-4-isopropoxy-benzene (40 mg, 0.250 mmol) was dissolved in dimethyl sulfoxide (DMSO) (2 mL) under Argon. The solution was degassed with argon for 30 min. This solution was transferred into the 3-neck flask via syringe and heated to 80° C. for 60 min. The reaction mixture was diluted with ethyl acetate and water. The mixture was stirred at room temperature for 10 mm. After phase separation with the organic layer was washed twice with water and once with saturated sodium chloride solution, dried (MgSO₄), filtered and evaporated. The crude product was purified by flash chromatography (silica 4 g, 0-10% MeOH in CH₂Cl₂, 18 mL/min) Yield: 65 mg (0.151 mmol, 88%, light brown oil).

In a 50 mL round bottom flask was methyl 1-((6′-((4-isopropoxyphenyl)ethynyl)-1′H-spiro[cyclopropane-1,4′-isoquinolin]-2′(3′H)-yl)methyl)cyclopropanecarboxylate (65 mg, 0.151 mmol) was dissolved in tetrahydrofuran (THF) (1 mL) and Methanol (MeOH) (1 mL). 2M aqueous NaOH solution (0.7 ml, 1.400 mmol) was added. The reaction mixture was stirred overnight. The solvent mixture was evaporated. The residue was treated in water and neutralized with 700 μL 2N aqeuous HCl. CH₂Cl₂ was added. After phase separation by passing the organic layer through a Chromabond-PTS-cartridge the organic layer was evaporated. The crude product was purified by flash chromatography (silica 4 g, 0-30% MeOH in CH₂Cl₂, 18 mL/min)

Yield: 24 mg (0.058 mmol, 38%, yellow foam).

Calculated mass (C₂₇H₂₉NO₃): 415.2; found mass: 416 (M+H⁺).

¹H NMR (600 MHz, DMSO-d₆) δ 7.49-7.41 (m, 2H), 7.25 (dd, J=7.8, 1.6 Hz, 1H), 7.13 (d, J=7.9 Hz, 1H), 6.99-6.89 (m, 2H), 6.89 (d, J=1.5 Hz, 1H), 4.67 (h, J=6.0 Hz, 1H), 3.95 (s, 2H), 2.78 (d, J=5.1 Hz, 4H), 1.27 (d, J=6.0 Hz, 7H), 1.17-1.01 (m, 4H), 0.96-0.90 (m, 2H), 0.75 (q, J=3.8 Hz, 2H). 29 protons.

The following compound was prepared in the same way as compound 8.

### 1-((6′-((6-(cyclopentyloxy)pyridin-3-yl)ethynyl)-1′H-spiro[cyclopropane-1,4′-isoquinolin]-2′(3′H)-yl)methyl)cyclopropanecarboxylic acid (Compound 9)

Calculated mass (C₂₈H₃₀N₂O₃): 442.2; found mass: 443 (M+H⁺).

¹H NMR (600 MHz, DMSO-d₆) δ 14.61 (s, 1H), 8.35 (d, J=2.3 Hz, 1H), 7.82 (dd, J=8.6, 2.4 Hz, 1H), 7.28 (dd, J=7.9, 1.6 Hz, 1H), 7.15 (d, J=7.9 Hz, 1H), 6.93 (d, J=1.6 Hz, 1H), 6.80 (d, J=8.5 Hz, 1H), 5.38 (tt, J=6.0, 2.8 Hz, 1H), 3.96 (s, 2H), 2.78 (d, J=6.0 Hz, 4H), 2.00-1.88 (m, 2H), 1.71 (dtd, J=8.0, 5.9, 5.3, 3.2 Hz, 4H), 1.66-1.54 (m, 2H), 1.14-1.01 (m, 4H), 0.93 (q, J=4.4 Hz, 2H), 0.75 (q, J=3.8 Hz, 2H). 30 protons.

### 3-(6′-((2,3-Difluorophenyl)ethynyl)-1′H-spiro[cyclopropane-1,4′-isoquinolin]-2′(3′H)-yl)propanoic acid (Compound 10)

Compound 10 was prepared from intermediate compound V according to reaction scheme 14.

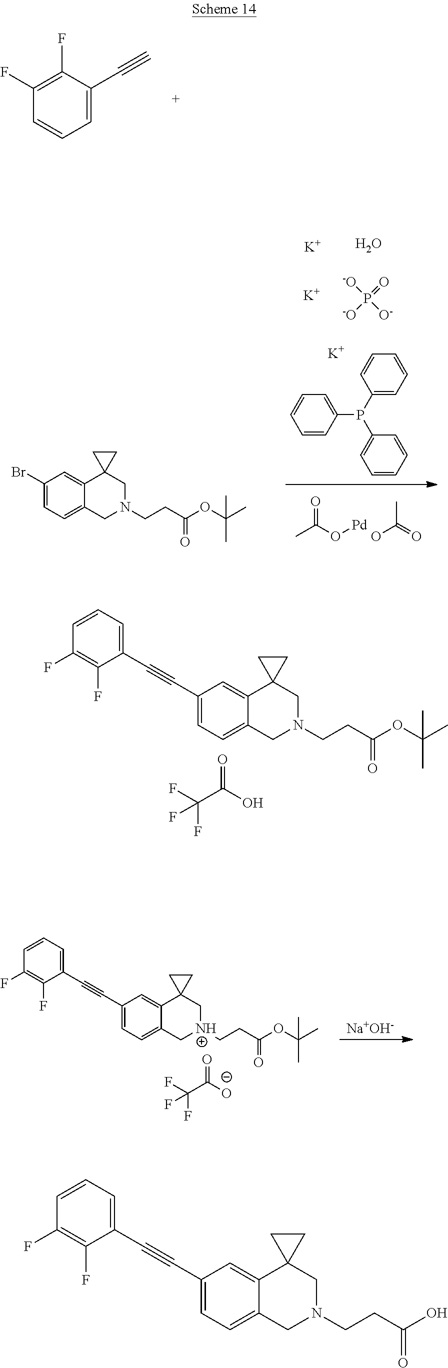

Tert-butyl 3-(6′-bromo-1′H-spiro[cyclopropane-1,4′-isoquinolin]-2′(3′H)-yl)propanoate (50 mg, 0.137 mmol) was dissolved in DMSO (1 mL) and 1-ethynyl-2,3-difluoro-benzene (28.3 mg, 0.205 mmol) was added. The solution was degassed with argon for 5 min, potassium phosphate (37.7 mg, 0.164 mmol), palladium acetate (1.8 mg, 8.2 μmol) and triphenylphosphine (7.2 mg, 0.027 mmol) were added. The reaction mixture was stirred at 80° C. for 1 h. The reaction mixture was cooled to room temperature, dichloromethane was added and the organic solution washed three times with water (60 mL total). The organic phase was dried (MgSO₄) and the solvent was evaporated. The crude product was purified by flash-chromatography (silica, dichloromethane/methanol) and preparative HPLC (WATERS XBRIDGE C18 OBD, acetonitrile, water, 0.1% trifluoro acetic acid).

Yield: 16.3 mg (0.030 mmol, 22%, light yellow oil).

Tert-butyl 3-(6′-((2,3-difluorophenyl)ethynyl)-1′H-spiro[cyclopropane-1,4′-isoquinolin]-2′(3′H)-yl)propanoate 2,2,2-trifluoroacetate (16.3 mg, 0.35 mmol) was dissolved in tetrahydrofuran (0.5 mL) and methanol (0.5 mL). Under stirring 1N aqueous sodium hydroxide (0.55 mL, 0.55 mmol) were added at room temperature. Stirring was continued overnight. The reaction mixture was neutralized with 1N aqueous hydrochloric acid (0.55 mL, 0.55 mmol) and the organic solvent was evaporated. Dichloromethane was added, the aqueous phase extracted with dichloromethane, the combined organic extracts were dried (MgSO₄) and the solvent evaporated. The product was dried under vacuum at 45° C.

Yield: 6.5 mg (0.018 mmol, 58%, colorless solid)

Calculated mass (C₂₂H₁₉F₂NO₂): 367.1; found mass: 368 (M+H⁺).

¹H NMR (600 MHz, DMSO-d₆) δ 7.52 (dtd, J=10.1, 8.1, 1.6 Hz, 1H), 7.45 (ddt, J=7.6, 6.0, 1.6 Hz, 1H), 7.31 (dd, J=7.9, 1.6 Hz, 1H), 7.27 (tdd, J=8.3, 5.1, 1.5 Hz, 1H), 7.15 (d, J=7.9 Hz, 1H), 6.94 (d, J=1.6 Hz, 1H), 3.94-3.78 (m, 2H), 2.80 (s, 2H), 2.69 (d, J=11.0 Hz, 2H), 1.05 (s, 2H), 0.96-0.90 (m, 2H).

Compound 11 was prepared in the same way as compound 10.

### 3-(6′-((2,6-dichlorophenyl)ethynyl)-1′H-spiro[cyclopropane-1,4′-isoquinolin]-2′(3′H)-yl)propanoic acid (Compound 11)

Calculated mass (C₂₂H₁₉Cl₂NO₂): 399.1; found mass: 400.4 (M+H⁺).

¹H NMR (600 MHz, DMSO-d6) δ 7.61 (d, J=8.2 Hz, 2H), 7.49-7.34 (m, 2H), 7.25 (d, J=7.9 Hz, 1H), 6.99 (d, J=1.6 Hz, 1H), 4.35 (s, 2H), 3.21 (s, 6H), 2.76 (t, J=7.5 Hz, 2H), 1.14 (d, J=53.0 Hz, 5H).

### General Procedure for Compounds 12 to 28

A 4 mL vial was charged with a stir bar to which was added NaH (13 mg, 0.52 mmol). To this NaH was then added a solution of ethyl 2-(6′-hydroxy-1′H-spiro[cyclopropane-1,4′-isoquinolin]-2′(3′H)-yl)acetate (intermediate compound II) (30 mg, 0.11 mmol) in dimethyl formamide (300 μl) at 0 degrees and stirred for 30 minutes. After 30 minutes, to this mixture was added a solution of halide monomer (1.4 eq., 0.16 mmol) also in dimethylformamide (200 ul). This was allowed to stir at rt for 2 hours. Upon completion of the first step, to the crude material was added 500 μl of 1M lithium hydroxide and further allowed to be stirred for 1 hour at room temperature. The residues were dissolved in DMSO and purified by reverse phase HPLC (TFA method). Samples were purified by preparative HPLC on a Phenomenex Luna C8(2) 5 um 100 Å AXIA column (30 mm×150 mm). A gradient of ACN (A) and 0.1% trifluoroacetic acid in water (B) was used, at a flow rate of 50 mL/min (0-0.5 min 5% A, 0.5-8.5 min linear gradient 5-100% A, 8.7-10.7 min 100% A, 10.7-11.0 min linear gradient 100-5% A) to obtain desired compounds. Product was characterized by ¹H NMR, MS and LC/MS.

For the preparation of compounds 12 and 13, ethyl 2-(6′-hydroxy-1′H-spiro[cyclopropane-1,4′-isoquinolin]-2′(3′H)-yl)acetate (intermediate compound II) was used. For the preparation of compounds 14 to 27, ethyl 4-(6′-hydroxy-1′H-spiro[cyclopropane-1,4′-isoquinolin]-2′(3′H)-yl)butanoate (intermediate compounds VII) was used instead of intermediate compound II. For the preparation of compound 28, ethyl 2-(6′-hydroxy-1′H-spiro[cyclopropane-1,4′-isoquinolin]-2′(3′H)-yl)propanoate (intermediate compound IX) was used instead of intermediate compound II.

### 2-(6′-((2,6-Dichlorobenzyl)oxy)-1′H-spiro[cyclopropane-1,4′-isoquinolin]-2′(3′H)-yl)acetic acid (Compound 12)

¹H NMR (400 MHz, Pyridine-d₅) δ 7.69 (d, J=7.2 Hz, 1H), 7.39-7.27 (m, 3H), 7.01 (d, J=8.4 Hz, 1H), 6.91 (dd, J=8.4, 2.6 Hz, 1H), 6.60 (d, J=2.5 Hz, 1H), 5.24 (s, 2H), 4.02 (s, 2H), 3.64 (s, 2H), 2.83 (s, 2H), 1.07-1.00 (m, 2H), 1.00-0.89 (m, 2H). MS (APCI) m/z 392.1 (M+H)⁺

### 2-(6′-((2-Chloro-6-ethylbenzyl)oxy)-1′H-spiro[cyclopropane-1,4′-isoquinolin]-2′(3′H)-yl)acetic acid (Compound 13)

¹H NMR (400 MHz, Pyridine-d₅) δ 7.36 (d, J=7.9 Hz, 1H), 7.24 (t, J=7.8 Hz, 1H), 7.15 (d, J=7.6 Hz, 1H), 7.05 (d, J=8.4 Hz, 1H), 6.98 (dd, J=8.4, 2.5 Hz, 1H), 6.63 (d, J=2.4 Hz, 1H), 5.29 (s, 2H), 4.05 (s, 2H), 3.66 (s, 2H), 2.85 (s, 2H), 2.72 (q, J=7.5 Hz, 2H), 1.15 (t, J=7.5 Hz, 3H), 1.07 (q, J=4.5 Hz, 2H), 0.94 (q, J=4.4 Hz, 2H). MS (APCI) m/z 386.1 (M+H)⁺

### 4-(6′-((2-(Trifluoromethoxy)benzyl)oxy)-1′H-spiro[cyclopropane-1,4′-isoquinolin]-2′(3′H)-yl)butanoic acid (compound 14)

¹H NMR (400 MHz, Pyridine-d₅) δ 7.72-7.65 (m, 1H), 7.36 (dd, J=4.8, 1.3 Hz, 2H), 7.34-7.27 (m, 1H), 7.02 (d, J=8.4 Hz, 1H), 6.92 (dd, J=8.3, 2.5 Hz, 1H), 6.58 (d, J=2.5 Hz, 1H), 5.24 (s, 2H), 3.70 (s, 2H), 2.68 (t, J=7.2 Hz, 2H), 2.57 (t, J=7.0 Hz, 2H), 2.51 (s, 2H), 2.08 (p, J=7.1 Hz, 2H), 1.04-0.96 (m, 2H), 0.92-0.84 (m, 2H). MS (APCI) m/z 436.3 (M+H)⁺

### 4-(6′-((2,6-Dichlorobenzyl)oxy)-1′H-spiro[cyclopropane-1,4′-isoquinolin]-2′(3′H)-yl)butanoic acid (Compound 15)

¹H NMR (400 MHz, Pyridine-d₅) δ 7.38 (d, J=7.9 Hz, 2H), 7.09-6.96 (m, 2H), 6.61 (d, J=2.5 Hz, 1H), 5.34 (s, 2H), 3.73 (s, 2H), 2.68 (t, J=7.2 Hz, 2H), 2.58 (t, J=7.0 Hz, 2H), 2.53 (s, 2H), 2.08 (p, J=7.1 Hz, 2H), 1.07-0.99 (m, 2H), 0.92-0.84 (m, 2H). MS (APCI) m/z 421.2 (M+H)⁺

### 4-(6′-((4-Chlorobenzyl)oxy)-1′H-spiro[cyclopropane-1,4′-isoquinolin]-2′(3′H)-yl)butanoic acid (Compound 16)

¹H NMR (400 MHz, Pyridine-d₅) δ 7.48-7.38 (m, 4H), 7.02 (d, J=8.5 Hz, 1H), 6.88 (dd, J=8.3, 2.5 Hz, 1H), 6.55 (d, J=2.6 Hz, 1H), 5.05 (s, 2H), 3.72 (s, 2H), 2.68 (t, J=7.3 Hz, 2H), 2.58 (t, J=7.0 Hz, 2H), 2.53 (s, 2H), 2.08 (p, J=7.2 Hz, 2H), 1.07-0.96 (m, 2H), 0.94-0.86 (m, 2H). MS (APCI) m/z 386.3 (M+H)⁺

### 4-(6′-((4-Fluorobenzyl)oxy)-1′H-spiro[cyclopropane-1,4′-isoquinolin]-2′(3′H)-yl)butanoic acid (Compound 17)

¹H NMR (400 MHz, Pyridine-d₅) δ 7.51-7.46 (m, 2H), 7.18-7.11 (m, 1H), 7.02 (d, J=8.4 Hz, 1H), 6.90 (dd, J=8.4, 2.5 Hz, 1H), 6.56 (d, J=2.5 Hz, 1H), 5.05 (s, 2H), 3.72 (s, 2H), 2.68 (t, J=7.3 Hz, 2H), 2.58 (t, J=7.0 Hz, 2H), 2.53 (s, 2H), 2.08 (p, J=7.2 Hz, 2H), 1.06-1.02 (m, 2H), 0.94-0.87 (m, 2H). MS (APCI) m/z 370.2 (M+H)⁺

### 4-(6′-((3,4-Difluorobenzyl)oxy)-1′H-spiro[cyclopropane-1,4′-isoquinolin]-2′(3′H)-yl)butanoic acid (Compound 18)

¹H NMR (400 MHz, Pyridine-d₅) δ 7.49-7.40 (m, 1H), 7.30-7.22 (m, 2H), 7.03 (d, J=8.4 Hz, 1H), 6.91-6.86 (m, 1H), 6.57 (d, J=2.5 Hz, 1H), 5.04 (s, 2H), 3.72 (s, 2H), 2.68 (t, J=7.3 Hz, 2H), 2.59 (t, J=7.0 Hz, 2H), 2.53 (s, 2H), 2.07 (q, J=7.1 Hz, 2H), 1.07-1.00 (m, 2H), 0.93-0.85 (m, 2H). MS (APCI) m/z 388.3 (M+H)⁺

### 4-(6′-((2-Fluorobenzyl)oxy)-1′H-spiro[cyclopropane-1,4′-isoquinolin]-2′(3′H)-yl)butanoic acid (Compound 19)

¹H NMR (400 MHz, Pyridine-d₅) δ 7.61 (dd, J=7.4, 1.8 Hz, 1H), 7.30 (tdd, J=7.2, 5.4, 2.7 Hz, 1H), 7.18-7.14 (m, 1H), 7.01 (d, J=8.4 Hz, 1H), 6.91 (dd, J=8.4, 2.5 Hz, 1H), 6.58 (d, J=2.5 Hz, 1H), 5.19 (s, 2H), 3.71 (s, 2H), 2.68 (t, J=7.2 Hz, 2H), 2.62-2.49 (m, 4H), 2.07 (p, J=7.2 Hz, 2H), 1.06-0.98 (m, 2H), 0.92-0.84 (m, 2H). MS (APCI) m/z 370.2 (M+H)⁺

### 4-(6′-((3-(Trifluoromethoxy)benzyl)oxy)-1′H-spiro[cyclopropane-1,4′-isoquinolin]-2′(3′H)-yl)butanoic acid (Compound 20)

¹H NMR (400 MHz, Pyridine-d₅) δ 7.58 (t, J=1.3 Hz, 2H), 7.52 (s, 1H), 7.50-7.36 (m, 2H), 7.02 (d, J=8.4 Hz, 1H), 6.91 (dd, J=8.4, 2.6 Hz, 1H), 6.58 (d, J=2.5 Hz, 1H), 5.12 (s, 2H), 3.71 (s, 2H), 2.68 (t, J=7.3 Hz, 2H), 2.62-2.50 (m, 4H), 2.08 (p, J=7.1 Hz, 2H), 1.07-0.99 (m, 2H), 0.93-0.86 (m, 2H). MS (APCI) m/z 436.3 (M+H)⁺

### 4-(6′-((2,3-Difluorobenzyl)oxy)-1′H-spiro[cyclopropane-1,4′-isoquinolin]-2′(3′H)-yl)butanoic acid (Compound 21)

¹H NMR (400 MHz, Pyridine-d₅) δ 7.35 (ddt, J=7.7, 6.1, 1.6 Hz, 1H), 7.21 (q, J=1.0 Hz, 1H), 7.14-6.99 (m, 2H), 6.92 (dd, J=8.4, 2.5 Hz, 1H), 6.59 (d, J=2.5 Hz, 1H), 5.20 (s, 2H), 3.71 (s, 2H), 2.68 (t, J=7.3 Hz, 2H), 2.58 (t, J=7.0 Hz, 2H), 2.53 (s, 2H), 2.08 (p, J=7.1 Hz, 2H), 1.07-0.99 (m, 2H), 0.93-0.85 (m, 2H). MS (APCI) m/z 388.3 (M+H)⁺

### 4-(6′-((2-Methylbenzyl)oxy)-1′H-spiro[cyclopropane-1,4′-isoquinolin]-2′(3′H)-yl)butanoic acid (Compound 22)

¹H NMR (400 MHz, Pyridine-d₅) δ 7.53 (dd, J=6.8, 2.1 Hz, 1H), 7.31-7.23 (m, 2H), 7.03 (d, J=8.4 Hz, 1H), 6.92 (dd, J=8.3, 2.5 Hz, 1H), 6.58 (d, J=2.5 Hz, 1H), 5.09 (s, 2H), 3.72 (s, 2H), 2.68 (t, J=7.3 Hz, 2H), 2.63-2.51 (m, 4H), 2.32 (s, 3H), 2.08 (p, J=7.2 Hz, 2H), 1.07-1.00 (m, 2H), 0.93-0.85 (m, 2H). MS (APCI) m/z 366.3 (M+H)⁺

### 4-(6′-((3-Fluorobenzyl)oxy)-1′H-spiro[cyclopropane-1,4′-isoquinolin]-2′(3′H)-yl)butanoic acid (Compound 23)

¹H NMR (400 MHz, Pyridine-d₅) δ 7.39-7.25 (m, 3H), 7.14-7.04 (m, 1H), 7.01 (d, J=8.4 Hz, 1H), 6.89 (dd, J=8.4, 2.6 Hz, 1H), 6.57 (d, J=2.5 Hz, 1H), 5.10 (s, 2H), 3.71 (s, 2H), 2.68 (t, J=7.3 Hz, 2H), 2.58 (t, J=7.0 Hz, 2H), 2.52 (s, 2H), 2.17-2.02 (m, 2H), 1.07-0.99 (m, 2H), 0.93-0.85 (m, 2H). MS (APCI) m/z 370.2 (M+H)⁺

### 4-(6′-((3-Chlorobenzyl)oxy)-1′H-spiro[cyclopropane-1,4′-isoquinolin]-2′(3′H)-yl)butanoic acid (compound 24)

¹H NMR (400 MHz, Pyridine-d₅) δ 7.63-7.55 (m, 1H), 7.42-7.31 (m, 2H), 7.28 (d, J=7.8 Hz, 1H), 7.01 (d, J=8.4 Hz, 1H), 6.89 (dd, J=8.4, 2.6 Hz, 1H), 6.57 (d, J=2.5 Hz, 1H), 5.07 (s, 2H), 3.71 (s, 2H), 2.68 (t, J=7.3 Hz, 2H), 2.58 (t, J=7.0 Hz, 2H), 2.52 (s, 2H), 2.08 (p, J=7.2 Hz, 2H), 1.07-1.00 (m, 2H), 0.93-0.86 (m, 2H). MS (APCI) m/z 386.3 (M+H)⁺

### 4-(6′-((2-Chlorobenzyl)oxy)-1′H-spiro[cyclopropane-1,4′-isoquinolin]-2′(3′H)-yl)butanoic acid (Compound 25)

¹H NMR (400 MHz, Pyridine-d₅) δ 7.69-7.62 (m, 1H), 7.48-7.40 (m, 1H), 7.26 (qd, J=7.2, 1.9 Hz, 2H), 7.02 (d, J=8.4 Hz, 1H), 6.91 (dd, J=8.4, 2.5 Hz, 1H), 6.59 (d, J=2.5 Hz, 1H), 5.22 (s, 2H), 3.71 (s, 2H), 2.68 (t, J=7.2 Hz, 2H), 2.58 (t, J=7.0 Hz, 2H), 2.52 (s, 2H), 2.08 (p, J=7.2 Hz, 2H), 1.06-0.99 (m, 2H), 0.92-0.85 (m, 2H). MS (APCI) m/z 386.3 (M+H)⁺

### 4-(6′-((3,4-Dichlorobenzyl)oxy)-1′H-spiro[cyclopropane-1,4′-isoquinolin]-2′(3′H)-yl)butanoic acid (Compound 26)

¹H NMR (400 MHz, Pyridine-d₅) δ 7.67 (d, J=2.1 Hz, 1H), 7.48 (d, J=8.2 Hz, 1H), 7.34 (dd, J=8.3, 2.1 Hz, 1H), 7.03 (d, J=8.4 Hz, 1H), 6.89 (dd, J=8.4, 2.5 Hz, 1H), 6.58 (d, J=2.6 Hz, 1H), 5.04 (s, 2H), 3.72 (s, 2H), 2.68 (t, J=7.3 Hz, 2H), 2.58 (t, J=7.0 Hz, 2H), 2.53 (s, 2H), 2.08 (p, J=7.1 Hz, 2H), 1.11-1.01 (m, 2H), 0.93-0.86 (m, 2H). MS (APCI) m/z 421.3 (M+H)⁺

### 4-(6′-((2-Chloro-6-ethylbenzyl)oxy)-1′H-spiro[cyclopropane-1,4′-isoquinolin]-2′(3′H)-yl)butanoic acid (Compound 27)

¹H NMR (400 MHz, Pyridine-d₅) δ 7.36 (dd, J=8.0, 1.3 Hz, 1H), 7.24 (t, J=7.8 Hz, 1H), 7.15 (dd, J=7.8, 1.2 Hz, 1H), 7.06 (d, J=8.4 Hz, 1H), 6.98 (dd, J=8.3, 2.5 Hz, 1H), 6.60 (d, J=2.5 Hz, 1H), 5.28 (s, 2H), 3.74 (s, 2H), 2.77-2.64 (m, 4H), 2.59 (t, J=7.0 Hz, 2H), 2.53 (s, 2H), 2.08 (p, J=7.1 Hz, 2H), 1.15 (t, J=7.5 Hz, 3H), 1.08-1.00 (m, 2H), 0.92-0.85 (m, 2H). MS (APCI) m/z 414.1 (M+H)⁺

### 2-(6′-((2-Chloro-6-ethylbenzyl)oxy)-1′H-spiro[cyclopropane-1,4′-isoquinolin]-2′(3′H)-yl)propanoic acid (Compound 28)

¹H NMR (400 MHz, Pyridine-d₅) δ 7.36 (dd, J=8.0, 1.3 Hz, 1H), 7.25 (d, J=7.7 Hz, 1H), 7.08 (d, J=8.5 Hz, 1H), 6.99 (dd, J=8.3, 2.5 Hz, 1H), 6.64 (d, J=2.4 Hz, 1H), 5.29 (s, 2H), 4.25-4.10 (m, 2H), 3.77 (q, J=6.9 Hz, 1H), 3.02-2.87 (m, 2H), 2.72 (q, J=7.6 Hz, 2H), 2.14 (s, 2H), 1.55 (d, J=7.0 Hz, 3H), 1.16 (q, J=7.2 Hz, 3H), 1.12-1.01 (m, 2H), 0.95 (t, J=4.3 Hz, 2H). MS (APCI) m/z 400.1 (M+H)⁺

### Ethyl 4-(6′-((4-(trifluoromethoxy)phenyl)ethynyl)-1′H-spiro[cyclopropane-1,4′-isoquinolin]-2′(3′H)-yl)butanoate 2,2,2-trifluoroacetate (Compound 29)

A 4 mL vial was charged with a stir bar to which was added ethyl 4-(6′-bromo-1′H-spiro[cyclopropane-1,4′-isoquinolin]-2′(3′H)-yl)butanoate (20 mg, 1 eq., 0.06 mmol) followed by 1-ethynyl-4-(trifluoromethoxy)benzene (16 mg, 1.5 eq., 0.09 mmol), copper Iodide (055 mg, 0.05 eq), 1,1′-Bis(diphenylphosphino)ferrocene]palladium(II) dichloride (4.5 mg, 0.1 eq, 0.01 mmol) and hunig's base (30 μl, 3 eq, 0.17 mmol) in 1.5 ml of THF, which was degassed and heated at 80 degrees for 15 hours overnight. The residues were dissolved in DMSO and purified by reverse phase HPLC (TFA method). Samples were purified by preparative HPLC on a Phenomenex Luna C8(2) 5 um 100 Å AXIA column (30 mm×150 mm). A gradient of ACN (A) and 0.1% trifluoroacetic acid in water (B) was used, at a flow rate of 50 mL/min (0-0.5 min 5% A, 0.5-8.5 min linear gradient 5-100% A, 8.7-10.7 min 100% A, 10.7-11.0 min linear gradient 100-5% A) to obtain desired compounds. Product was characterized by ¹H NMR, MS and LC/MS.

¹H NMR (400 MHz, DMSO-d₆) δ 7.72-7.67 (m, 2H), 7.48-7.37 (m, 3H), 7.28 (d, J=8.0 Hz, 1H), 7.07 (s, 1H), 4.50 (s, 2H), 4.09 (q, J=7.1 Hz, 2H), 3.19 (s, 2H), 2.44 (t, J=7.2 Hz, 2H), 1.99 (t, J=8.2 Hz, 2H), 1.19 (q, J=6.9 Hz, 8H). MS (APCI) m/z 458.3 (M+H)⁺

### 3-(6′-((2,6-dichlorophenyl)ethynyl)-1′H-spiro[cyclopropane-1,4′-isoquinolin]-2′(3′H)-yl)propanoic acid acetic acid salt (1:1) (Compound 30)

A microwave vial was charged with K₃PO₄ (50.7 mg, 2.5 eq, 0.24 mmol), XPHOS (10.93 mg, 24 mol %, 0.02 mmol), PdCl₂(MeCN)₂ (1.86 mg, 7.5 mol %, 0.01 mmol), and stir bar. This was capped with a septa crimp cap and placed under N₂ to purge for a couple of minutes. To this, via syringe, 500 μL of a solution of tert-butyl 3-(6′-bromo-1′H-spiro[cyclopropane-1,4′-isoquinolin]-2′(3′H)-yl)propanoate in dry Acetonitrile (35.0 mg, 0.1 mmol) and 318.5 μL of a solution of 1,3-dichloro-2-ethynylbenzene that was prepared from a pre-weighed vial containing 0.6 mmol of substrate dissolved in 1000 μL of dry Acetonitrile (32.7 mg, 2 eq, 0.2 mmol) was added. The vial was left to purge for a couple of minutes more under N₂ and then placed to heat at 85° C. for 4 hours until reaction is complete. Upon completion the compound was filtered using a Celite cartridge washing 2 times with MeOH. This is then dried under a N₂ blower. The dried crude material is then dissolved in 1000 μL of an aqueous solution of 1M LiOH in 75% MeOH. This was capped and left to stir at 60° C. for 1 hour. Upon completion the crude material was filtered once more in a celite cartridge and washed 2 times with 1000 μL of Acetonitrile. The crude material is dried once more under N2 blower and then dissolved in 1400 μL of DMSO/Acetonitrile. This was then submitted to APS for reverse phase HPLC purification using AA method described above to yield, 3-(6′-((2,6-dichlorophenyl)ethynyl)-1′H-spiro[cyclopropane-1,4′-isoquinolin]-2′(3′H)-yl)propanoic acid (6.7 mg, 17%).

¹H NMR (400 MHz, Pyridine-d₅) δ 7.51 (dd, J=7.8, 1.6 Hz, 1H), 7.40 (s, 1H), 7.38 (s, 1H), 7.18-7.13 (m, 1H), 7.08 (d, J=7.9 Hz, 1H), 3.83 (s, 2H), 3.02 (t, J=7.3 Hz, 2H), 2.84 (t, J=7.1 Hz, 2H), 2.60 (s, 2H), 1.04-0.96 (m, 2H), 0.93-0.86 (m, 2H).

m/z 399.9 (M+H)⁺

The following compounds were prepared in the same way as compound 30.

### 3-(6′-((4-isopropylphenyl)ethynyl)-1′H-spiro[cyclopropane-1,4′-isoquinolin]-2′(3′H)-yl)propanoic acid acetic acid salt (1:1) (Compound 31)

¹H NMR (400 MHz, Pyridine-d₅) δ 7.68-7.65 (m, 2H), 7.44-7.42 (m, 1H), 7.25 (s, 1H), 7.23 (s, 1H), 7.18 (d, J=1.6 Hz, 1H), 7.03 (d, J=7.8 Hz, 1H), 7.00 (s, 1H), 3.81 (s, 2H), 3.01 (t, J=7.1 Hz, 2H), 2.86-2.75 (m, 3H), 2.60 (s, 2H), 1.14 (d, J=6.9 Hz, 6H), 1.05-0.99 (m, 2H), 0.92 (t, J=3.1 Hz, 2H).

m/z 374.1 (M+H)⁺

### 3-(6′-((3,5-difluorophenyl)ethynyl)-1′H-spiro[cyclopropane-1,4′-isoquinolin]-2′(3′H)-yl)propanoic acid acetic acid salt (1:1) (Compound 32)

¹H NMR (400 MHz, Pyridine-d₅) δ 7.42 (dd, J=7.9, 1.6 Hz, 1H), 7.19 (d, J=1.6 Hz, 1H), 7.07 (dd, J=8.9, 2.5 Hz, 2H), 7.05-7.01 (m, 1H), 7.00 (s, 1H), 3.83 (s, 2H), 3.02 (t, J=7.1 Hz, 2H), 2.84 (t, J=7.1 Hz, 2H), 2.61 (s, 2H), 1.08-1.02 (m, 2H), 0.98-0.90 (m, 2H).

m/z 368 (M+H)⁺

### 3-(6′-((3-chlorophenyl)ethynyl)-1′H-spiro[cyclopropane-1,4′-isoquinolin]-2′(3′H)-yl)propanoic acid acetic acid salt (1:1) (Compound 33)

1H NMR (400 MHz, Pyridine-d₅) δ 7.64 (t, J=1.8 Hz, 1H), 7.50 (dt, J=7.6, 1.3 Hz, 2H), 7.43 (dd, J=7.8, 1.6 Hz, 1H), 7.36-7.33 (m, 1H), 7.26 (t, J=7.9 Hz, 1H), 7.20-7.19 (m, 1H), 7.06 (d, J=7.9 Hz, 1H), 3.83 (s, 2H), 3.01 (t, J=7.1 Hz, 2H), 2.83 (t, J=7.2 Hz, 2H), 2.61 (s, 2H), 1.10-1.02 (m, 2H), 0.98-0.90 (m, 3H).

m/z 365.9 (M+H)⁺

### 3-(6′-((4-ethoxyphenyl)ethynyl)-1′H-spiro[cyclopropane-1,4′-isoquinolin]-2′(3′H)-yl)propanoic acid acetic acid salt (1:1) (Compound 34)

¹H NMR (400 MHz, Pyridine-d₅) δ 7.70-7.61 (m, 2H), 7.43 (dd, J=7.8, 1.6 Hz, 1H), 7.17 (d, J=1.5 Hz, 1H), 7.03 (d, J=7.8 Hz, 1H), 7.04-6.95 (m, 3H), 3.90 (q, J=7.0 Hz, 2H), 3.81 (s, 2H), 3.00 (t, J=7.1 Hz, 2H), 2.83 (t, J=7.1 Hz, 2H), 2.60 (s, 2H), 1.25 (t, J=7.0 Hz, 3H), 1.06-1.01 (m, 2H), 0.94-0.89 (m, 2H).

m/z 376 (M+H)⁺

### 3-(6′-((2-chlorophenyl)ethynyl)-1′H-spiro[cyclopropane-1,4′-isoquinolin]-2′(3′H)-yl)propanoic acid (Compound 35)

¹H NMR (400 MHz, Pyridine-d₅) δ 7.67-7.65 (m, 1H), 7.51-7.48 (m, 1H), 7.47-7.45 (m, 1H), 7.25-7.22 (m, 3H), 7.18 (d, J=1.6 Hz, 1H), 7.05 (d, J=7.9 Hz, 1H), 3.82 (s, 2H), 3.01 (t, J=7.1 Hz, 2H), 2.83 (t, J=7.1 Hz, 2H), 2.59 (s, 2H), 1.03-0.98 (m, 2H), 0.93-0.88 (m, 2H).

m/z 366 (M+H)⁺

### 3-(6′-((2,6-difluorophenyl)ethynyl)-1′H-spiro[cyclopropane-1,4′-isoquinolin]-2′(3′H)-yl)propanoic acid acetic acid salt (1:1) Compound 36)

¹H NMR (400 MHz, Pyridine-d₅) δ 7.43 (dd, J=7.8, 1.7 Hz, 1H), 7.30-7.23 (m, 1H), 7.16 (d, J=1.5 Hz, 1H), 7.05-6.99 (m, 3H), 3.80 (s, 2H), 3.01 (t, J=7.1 Hz, 2H), 2.83 (t, J=7.1 Hz, 2H), 2.58 (s, 2H), 1.01-0.95 (m, 2H), 0.91-0.85 (m, 2H).

m/z 368 (M+H)⁺

### 3-(6′-(o-tolylethynyl)-1′H-spiro[cyclopropane-1,4′-isoquinolin]-2′(3′H)-yl)propanoic acid acetic acid salt (1:1) (Compound 37)

¹H NMR (400 MHz, Pyridine-d₅) δ 7.65 (d, J=7.3 Hz, 1H), 7.44 (dd, J=7.8, 1.7 Hz, 1H), 7.29-7.22 (m, 2H), 7.17 (d, J=1.6 Hz, 1H), 7.06 (d, J=7.9 Hz, 1H), 3.83 (s, 2H), 3.01 (t, J=7.1 Hz, 2H), 2.84 (d, J=7.0 Hz, 2H), 2.61 (s, 2H), 2.50 (s, 3H), 1.06-1.02 (m, 2H), 0.94-0.90 (m, 2H).

m/z 346 (M+H)⁺

### 3-(6′-(thiophen-3-ylethynyl)-1′H-spiro[cyclopropane-1,4′-isoquinolin]-2′(3′H)-yl)propanoic acid (Compound 38)

¹H NMR (400 MHz, Pyridine-d₅) δ 7.84 (dd, J=3.0, 1.2 Hz, 1H), 7.48 (dd, J=5.0, 3.0 Hz, 1H), 7.39 (dd, J=7.9, 1.4 Hz, 1H), 7.36 (dd, J=5.0, 1.2 Hz, 1H), 7.13 (d, J=1.5 Hz, 1H), 7.02 (d, J=8.1 Hz, 1H), 3.81 (s, 2H), 3.00 (t, J=7.1 Hz, 2H), 2.83 (t, J=7.1 Hz, 2H), 2.59 (s, 2H), 1.03-0.98 (m, 2H), 0.94-0.88 (m, 2H).

m/z 337.9 (M+H)⁺

### 3-(6′-(phenylethynyl)-1′H-spiro[cyclopropane-1,4′-isoquinolin]-2′(3′H)-yl)propanoic acid acetic acid salt (1:1) (Compound 39)

¹H NMR (400 MHz, Pyridine-d₅) δ 7.69-7.65 (m, 2H), 7.43-7.40 (m, 2H), 7.39-7.32 (m, 3H), 7.17 (d, J=1.6 Hz, 1H), 7.03 (d, J=7.8 Hz, 1H), 7.03-6.98 (m, 1H), 3.81 (s, 2H), 3.01 (t, J=7.1 Hz, 2H), 2.83 (t, J=7.1 Hz, 2H), 2.60 (s, 2H), 1.04-1.00 (m, 2H), 0.94-0.88 (m, 2H).

m/z 332 (M+H)⁺

### 3-(6′-((6-chloropyridin-2-yl)ethynyl)-1′H-spiro[cyclopropane-1,4′-isoquinolin]-2′(3′H)-yl)propanoic acid acetic acid salt (1:1) (Compound 40)

¹H NMR (400 MHz, Pyridine-d₅) δ 7.62 (d, J=7.8 Hz, 1H), 7.51 (dd, J=7.6, 0.9 Hz, 1H), 7.42 (s, 1H), 7.31 (dd, J=7.9, 0.9 Hz, 1H), 7.17 (d, J=1.6 Hz, 1H), 7.01 (d, J=2.3 Hz, 1H), 3.81 (s, 2H), 3.02-2.96 (m, 2H), 2.85-2.79 (m, 2H), 2.59 (s, 2H), 1.05-1.01 (m, 2H), 0.95-0.90 (m, 2H).

m/z 366.9 (M+H)⁺

### 3-(6′-((4-fluorophenyl)ethynyl)-1′H-spiro[cyclopropane-1,4′-isoquinolin]-2′(3′H)-yl)propanoic acid acetic acid salt (1:1) (compound 41)

¹H NMR (400 MHz, Pyridine-d₅) δ 7.62 (dd, J=8.7, 5.5 Hz, 2H), 7.42 (dd, J=7.8, 1.6 Hz, 1H), 7.17 (d, J=2.1 Hz, 2H), 7.15-7.11 (m, 1H), 7.04 (d, J=7.8 Hz, 1H), 3.82 (s, 2H), 3.01 (t, J=7.1 Hz, 2H), 2.83 (t, J=7.1 Hz, 2H), 2.60 (s, 2H), 1.07-1.01 (m, 2H), 0.95-0.90 (m, 2H).

m/z 350 (M+H)⁺

### 3-(6′-((1-propyl-1H-pyrazol-4-yl)ethynyl)-1′H-spiro[cyclopropane-1,4′-isoquinolin]-2′(3′H)-yl)propanoic acid acetic acid salt (1:1) (Compound 42)

¹H NMR (400 MHz, Pyridine-d₅) δ 8.00 (dd, J=7.2, 0.7 Hz, 2H), 7.40 (dd, J=7.8, 1.6 Hz, 1H), 7.13 (d, J=1.6 Hz, 1H), 7.02 (d, J=7.9 Hz, 1H), 4.02 (t, J=6.9 Hz, 2H), 3.81 (s, 2H), 3.00 (t, J=7.1 Hz, 2H), 2.82 (t, J=7.0 Hz, 2H), 2.59 (s, 2H), 1.77 (h, J=7.3 Hz, 2H), 1.04-0.99 (m, 2H), 0.94-0.89 (m, 2H), 0.74 (t, J=7.4 Hz, 3H).

m/z 364 (M+H)⁺

### 3-(6′-((3-methylpyridin-2-yl)ethynyl)-1′H-spiro[cyclopropane-1,4′-isoquinolin]-2′(3′H)-yl)propanoic acid acetic acid salt (1:1) (Compound 43)

¹H NMR (400 MHz, Pyridine-d₅) δ 8.60 (dd, J=4.8, 1.6 Hz, 1H), 7.48-7.45 (m, 1H), 7.43 (dd, J=7.8, 1.6 Hz, 1H), 7.15 (d, J=1.6 Hz, 1H), 7.12 (dd, J=7.8, 4.7 Hz, 1H), 7.03 (d, J=7.9 Hz, 1H), 3.81 (s, 2H), 3.02 (t, J=7.1 Hz, 2H), 2.84 (t, J=7.1 Hz, 2H), 2.59 (s, 2H), 2.42 (s, 3H), 1.02-0.98 (m, 2H), 0.93-0.88 (m, 2H).

m/z 347 (M+H)⁺

### 4-(6′-((4-(cyclopentyloxy)phenyl)ethynyl)-1′H-spiro[cyclopropane-1,4′-isoquinolin]-2′(3′H)-yl)butanoic acid (Compound 44)

A microwave vial was charged with K₃PO₄ (36.6 mg, 2.5 eq, 0.17 mmol), XPHOS (7.9 mg, 24 mol %, 0.02 mmol), PdCl₂(MeCN)₂ (1.34 mg, 7.5 mol %, 0.01 mmol), and stir bar. This was capped with a septa crimp cap and placed under N₂ to purge for a couple of minutes. To this, via syringe, 500 μL of a solution of ethyl 4-(6′-bromo-1′H-spiro[cyclopropane-1,4′-isoquinolin]-2′(3′H)-yl)butanoate in dry Acetonitrile (24.3 mg, 0.07 mmol) and 230 μL of a solution of 1-(cyclopentyloxy)-4-ethynylbenzene that was prepared from a pre-weighed vial containing 0.6 mmol of substrate dissolved in 1000 μL of dry Acetonitrile (25.7 mg, 2 eq, 0.14 mmol) was added. The vial was left to purge for a couple of minutes more under N₂ and then placed to heat at 85° C. for 4 hours until reaction is complete. Upon completion the compound was filtered using a Celite cartridge washing 2 times with MeOH. This is then dried under a N₂ blower. The dried crude material is then dissolved in 1000 μL of an aqueous solution of 1M LiOH in 75% MeOH. This was capped and left to stir at 60° C. for 1 hour. Upon completion the crude material was filtered once more in a celite cartridge and washed 2 times with 1000 μL of Acetonitrile. The crude material is dried once more under N₂ blower and then dissolved in 1400 μL of DMSO/Acetonitrile. The crude product was purified by reverse phase HPLC using the AA method to yield, ethyl 4-(6′-((4-(cyclopentyloxy)phenyl)ethynyl)-1′H-spiro[cyclopropane-1,4′-isoquinolin]-2′(3′H)-yl)butanoate (8.4 mg, 31%).

¹H NMR (400 MHz, Pyridine-d₅) δ 7.70-7.65 (m, 2H), 7.44 (dd, J=7.9, 1.6 Hz, 1H), 7.18 (d, J=1.6 Hz, 1H), 7.05 (d, J=7.9 Hz, 1H), 7.03-6.99 (m, 2H), 4.74-4.68 (m, 1H), 3.73 (s, 2H), 2.67 (t, J=7.3 Hz, 2H), 2.57 (t, J=7.0 Hz, 2H), 2.51 (s, 2H), 2.07 (p, J=7.2 Hz, 2H), 1.82-1.75 (m, 5H), 1.71-1.62 (m, 2H), 1.53-1.43 (m, 2H), 1.06-1.01 (m, 2H), 0.93-0.87 (m, 2H).

m/z 430.3 (M+H)⁺

The following compounds were prepared in the same way as compound 44.

### 4-(6′-((4-ethoxy-3-fluorophenyl)ethynyl)-1′H-spiro[cyclopropane-1,4′-isoquinolin]-2′(3′H)-yl)butanoic acid (compound 45)

¹H NMR (400 MHz, Pyridine-d₅) δ 7.50 (dd, J=12.0, 2.0 Hz, 1H), 7.45-7.42 (m, 1H), 7.18 (d, J=1.6 Hz, 1H), 7.17-7.10 (m, 1H), 7.06 (d, J=7.9 Hz, 1H), 7.00 (dd, J=8.3, 2.4 Hz, 1H), 4.02-3.92 (m, 2H), 2.67 (td, J=7.3, 4.0 Hz, 2H), 2.57 (q, J=6.9 Hz, 2H), 2.52 (s, 1H), 2.13-2.02 (m, 2H), 1.27 (td, J=7.0, 4.5 Hz, 3H), 1.06-1.02 (m, 2H), 0.95-0.89 (m, 2H).

m/z 408.3 (M+H)⁺

### 4-(6′-((4-isopropoxyphenyl)ethynyl)-1′H-spiro[cyclopropane-1,4′-isoquinolin]-2′(3′H)-yl)butanoic acid (Compound 46)

¹H NMR (400 MHz, Pyridine-d₅) δ 7.69-7.65 (m, 2H), 7.43 (dd, J=7.9, 1.6 Hz, 1H), 7.17 (d, J=1.6 Hz, 1H), 7.04-7.00 (m, 3H), 4.50 (dq, J=10.5, 6.0 Hz, 1H), 3.73 (s, 2H), 2.67 (dd, J=8.1, 6.4 Hz, 2H), 2.60-2.54 (m, 2H), 2.51 (s, 2H), 2.08 (p, J=7.3 Hz, 2H), 1.23 (dd, J=6.0, 2.4 Hz, 6H), 1.04-1.01 (m, 2H), 0.89 (d, J=2.1 Hz, 2H).

m/z 404.3 (M+H)⁺

### 4-(6′-((3-fluoro-4-(trifluoromethoxy)phenyl)ethynyl)-1′H-spiro[cyclopropane-1,4′-isoquinolin]-2′(3′H)-yl)butanoic acid (Compound 47)

¹H NMR (400 MHz, Pyridine-d₅) δ 7.55 (d, J=1.9 Hz, 1H), 7.43 (dddd, J=8.1, 5.9, 4.4, 1.7 Hz, 3H), 7.19 (d, J=1.5 Hz, 1H), 7.08 (d, J=7.9 Hz, 1H), 3.75 (s, 2H), 2.68 (t, J=7.3 Hz, 2H), 2.59 (t, J=7.1 Hz, 2H), 2.53 (s, 2H), 2.10 (dd, J=14.3, 7.1 Hz, 2H), 1.06-1.02 (m, 2H), 0.96-0.88 (m, 2H).

m/z 448.3 (M+H)⁺

### 4-(6′-((4-(trifluoromethoxy)phenyl)ethynyl)-1′H-spiro[cyclopropane-1,4′-isoquinolin]-2′(3′H)-yl)butanoic acid (Compound 4)8

¹H NMR (400 MHz, Pyridine-d₅) δ 7.70-7.65 (m, 2H), 7.43 (dd, J=7.8, 1.6 Hz, 1H), 7.33-7.29 (m, 2H), 7.18 (d, J=1.5 Hz, 1H), 7.07 (d, J=7.9 Hz, 1H), 3.74 (s, 2H), 2.68 (t, J=7.3 Hz, 2H), 2.59 (t, J=7.1 Hz, 2H), 2.52 (s, 2H), 2.10 (dd, J=15.4, 8.3 Hz, 2H), 1.05-1.01 (m, 2H), 0.94-0.88 (m, 2H).

m/z 430.3 (M+H)⁺

### 4-(6′-((6-ethoxy-4-methylpyridin-3-yl)ethynyl)-1′H-spiro[cyclopropane-1,4′-isoquinolin]-2′(3′H)-yl)butanoic acid (Compound 49)

¹H NMR (400 MHz, Pyridine-d₅) δ 8.54 (s, 1H), 7.45 (dd, J=7.8, 1.6 Hz, 1H), 7.19 (d, J=1.6 Hz, 1H), 7.09 (d, J=7.9 Hz, 1H), 6.71 (s, 1H), 4.42 (q, J=7.1 Hz, 2H), 3.75 (s, 2H), 2.67 (t, J=7.3 Hz, 2H), 2.58 (t, J=7.0 Hz, 2H), 2.53 (s, 2H), 2.38 (s, 3H), 2.08 (q, J=7.1 Hz, 2H), 1.30 (t, J=7.1 Hz, 3H), 1.08-1.03 (m, 2H), 0.93-0.88 (m, 2H).

m/z 405.2 (M+H)⁺

### 4-(6′-((6-ethoxypyridin-3-yl)ethynyl)-1′H-spiro[cyclopropane-1,4′-isoquinolin]-2′(3′H)-yl)butanoic acid (Compound 50)

m/z 391.2 (M+H)⁺

### 4-(6′-((6-ethoxy-5-fluoropyridin-3-yl)ethynyl)-1′H-spiro[cyclopropane-1,4′-isoquinolin]-2′(3′H)-yl)butanoic acid (Compound 51)

¹H NMR (400 MHz, Pyridine-d₅) δ 8.32 (d, J=1.9 Hz, 1H), 7.71 (dd, J=10.9, 1.9 Hz, 1H), 7.43 (dd, J=7.8, 1.6 Hz, 1H), 7.18 (d, J=1.6 Hz, 1H), 7.08 (d, J=7.9 Hz, 1H), 4.43 (q, J=7.1 Hz, 2H), 3.76 (s, 2H), 2.67 (t, J=7.2 Hz, 2H), 2.59 (t, J=7.0 Hz, 2H), 2.54 (s, 2H), 2.12-2.03 (m, 2H), 1.30 (t, J=7.1 Hz, 3H), 1.06-1.02 (m, 2H), 0.95-0.90 (m, 2H).

m/z 409.2 (M+H)⁺

### 4-(6′-((5-isopropoxypyridin-2-yl)ethynyl)-1′H-spiro[cyclopropane-1,4′-isoquinolin]-2′(3′H)-yl)butanoic acid (Compound 52)

¹H NMR (400 MHz, Pyridine-d₅) δ 8.58 (d, J=2.9 Hz, 1H), 7.64 (d, J=8.6 Hz, 1H), 7.42 (dd, J=7.9, 1.6 Hz, 1H), 7.29 (dd, J=8.7, 3.0 Hz, 1H), 7.16 (d, J=1.6 Hz, 1H), 7.02 (d, J=8.1 Hz, 1H), 4.55 (p, J=6.0 Hz, 1H), 3.71 (s, 2H), 2.67 (t, J=7.2 Hz, 2H), 2.57 (t, J=7.0 Hz, 2H), 2.50 (s, 2H), 2.07 (p, J=7.2 Hz, 2H), 1.23 (d, J=6.0 Hz, 6H), 1.01-0.96 (m, 2H), 0.91-0.87 (m, 2H).

m/z 405.3 (M+H)⁺

### 4-(6′-((6-ethoxy-2-methylpyridin-3-yl)ethynyl)-1′H-spiro[cyclopropane-1,4′-isoquinolin]-2′(3′H)-yl)butanoic acid (Compound 53)

1H NMR (400 MHz, Pyridine-d₅) δ 7.76 (d, J=8.5 Hz, 1H), 7.45 (dd, J=7.8, 1.6 Hz, 1H), 7.20-7.18 (m, 1H), 7.09 (d, J=7.9 Hz, 1H), 6.69 (d, J=8.3 Hz, 1H), 4.40 (q, J=7.1 Hz, 2H), 3.76 (s, 2H), 2.73 (s, 3H), 2.67 (t, J=7.3 Hz, 2H), 2.59 (t, J=7.0 Hz, 2H), 2.53 (s, 2H), 2.09 (q, J=7.1 Hz, 2H), 1.30 (t, J=7.1 Hz, 3H), 1.09-1.04 (m, 2H), 0.94-0.89 (m, 2H).

m/z 405.2 (M+H)⁺

### 4-(6′-((5-ethoxypyridin-2-yl)ethynyl)-1′H-spiro[cyclopropane-1,4′-isoquinolin]-2′(3′H)-yl)butanoic acid (Compound 54)

¹H NMR (400 MHz, Pyridine-d₅) δ 8.56 (d, J=3.0 Hz, 1H), 7.63 (d, J=8.6 Hz, 1H), 7.42 (dd, J=7.8, 1.6 Hz, 1H), 7.24 (dd, J=8.7, 3.0 Hz, 1H), 7.16 (d, J=1.6 Hz, 1H), 7.02 (d, J=7.9 Hz, 1H), 3.95 (t, J=7.0 Hz, 2H), 3.71 (s, 2H), 2.66 (t, J=7.3 Hz, 2H), 2.56 (t, J=7.0 Hz, 2H), 2.50 (s, 2H), 2.06 (p, J=7.2 Hz, 2H), 1.26 (t, J=6.9 Hz, 4H), 1.04-0.96 (m, 2H), 0.92-0.87 (m, 2H).

m/z 391.2 (M+H)⁺

### 4-(6′-((5-fluoro-6-methoxypyridin-3-yl)ethynyl)-1′H-spiro[cyclopropane-1,4′-isoquinolin]-2′(3′H)-yl)butanoic acid (Compound 55)

¹H NMR (400 MHz, Pyridine-d₅) δ 8.32 (d, J=1.9 Hz, 1H), 7.70 (dd, J=10.9, 1.9 Hz, 1H), 7.43 (d, J=1.6 Hz, 1H), 7.17 (d, J=1.6 Hz, 1H), 7.07 (d, J=7.9 Hz, 1H), 7.00 (s, 1H), 3.95 (s, 3H), 3.75 (s, 2H), 2.68 (t, J=7.3 Hz, 2H), 2.59 (t, J=7.1 Hz, 2H), 2.53 (s, 2H), 2.10 (t, J=7.1 Hz, 2H), 1.06-1.02 (m, 2H), 0.93-0.89 (m, 2H).

m/z 395.3 (M+H)⁺

### 4-(6′-(benzo[d][1,3]dioxol-5-ylethynyl)-1′H-spiro[cyclopropane-1,4′-isoquinolin]-2′(3′H)-yl)butanoic acid (Compound 56)

m/z 390.2 (M+H)⁺

### 4-(6′-((6-methoxy-2-methylpyridin-3-yl)ethynyl)-1′H-spiro[cyclopropane-1,4′-isoquinolin]-2′(3′H)-yl)butanoic acid (compound 57)

¹H NMR (400 MHz, Pyridine-d₅) δ 7.75 (d, J=8.5 Hz, 1H), 7.45 (dd, J=7.8, 1.6 Hz, 1H), 7.19 (d, J=1.7 Hz, 2H), 7.09 (d, J=7.9 Hz, 1H), 6.69 (d, J=8.5 Hz, 1H), 3.92 (s, 3H), 3.76 (s, 2H), 2.72 (s, 3H), 2.67 (t, J=7.3 Hz, 2H), 2.58 (t, J=7.0 Hz, 2H), 2.53 (s, 2H), 2.09 (q, J=7.2 Hz, 2H), 1.09-1.03 (m, 2H), 0.94-0.88 (m, 2H).

m/z 391.2 (M+H)⁺

### 4-(6′-((2,6-difluorophenyl)ethynyl)-1′H-spiro[cyclopropane-1,4′-isoquinolin]-2′(3′H)-yl)-3-methylbutanoic acid (Compound 58)

A microwave vial was charged with K₃PO₄ (69.54 mg, 3 eq, 0.33 mmol), XPHOS (12.52 mg, 24 mol %, 0.03 mmol), PdCl₂(MeCN)₂ (4.25 mg, 15 mol %, 0.02 mmol), and stir bar. This was capped with a septa crimp cap and placed under N₂ to purge for a couple of minutes. To this, via syringe, 500 μL of a solution of ethyl 4-(6′-bromo-1′H-spiro[cyclopropane-1,4′-isoquinolin]-2′(3′H)-yl)-3-methylbutanoate in dry Acetonitrile (40 mg, 0.11 mmol) and 365 μL of a solution 2-ethoxy-5-ethynyl-4-methylpyridine that was prepared from a pre-weighed vial containing 0.6 mmol of substrate dissolved in 1000 μL of dry Acetonitrile (35.21 mg, 1.5 eq, 0.09 mmol) was added. The vial was left to purge for a couple of minutes more under N₂ and then placed to heat at 100° C. for 18 hours until reaction is complete. Upon completion the compound was filtered using a Celite cartridge washing 2 times with MeOH. This is then dried under a N₂ blower. The dried crude material is then dissolved in 500 μL of THF and 1500 μL of an aqueous solution of 1M LiOH in 75% MeOH. This was capped and left to stir at 60° C. for 2 hour. Upon completion the crude material was filtered once more in a celite cartridge and washed 2 times with 1000 μL of Acetonitrile. The crude material is dried once more under N2 blower and then dissolved in 1400 μL of DMSO/Acetonitrile. The crude product was purified by reverse phase HPLC using the AA method described above to yield 4-(6′-((6-ethoxy-4-methylpyridin-3-yl)ethynyl)-1′H-spiro[cyclopropane-1,4′-isoquinolin]-2′(3′H)-yl)-3-methylbutanoic acid (16.7 mg, 38.51%).

¹H NMR (400 MHz, Pyridine-d₅) δ 7.44 (dd, J=7.8, 1.6 Hz, 1H), 7.32-7.24 (m, 1H), 7.16 (d, J=1.6 Hz, 1H), 7.06 (d, J=3.4 Hz, 1H), 7.04 (d, J=3.0 Hz, 1H), 7.02 (d, J=7.5 Hz, 1H), 3.82-3.68 (m, 2H), 2.86 (dd, J=15.1, 5.0 Hz, 1H), 2.57-2.45 (m, 3H), 2.41-2.27 (m, 3H), 1.14 (d, J=6.4 Hz, 3H), 1.00-0.83 (m, 4H).

m/z 396.2 (M+H)⁺

The following compounds were prepared in the same way as compound 58.

### 4-(6′-((6-isopropoxypyridin-3-yl)ethynyl)-1′H-spiro[cyclopropane-1,4′-isoquinolin]-2′(3′H)-yl)-3-methylbutanoic acid (Compound 59)

¹H NMR (400 MHz, DMSO-d₆) δ 8.33 (dd, J=2.4, 0.8 Hz, 1H), 7.82 (dd, J=8.6, 2.4 Hz, 1H), 7.24 (dd, J=7.8, 1.6 Hz, 1H), 7.11 (d, J=8.0 Hz, 1H), 6.87 (d, J=1.6 Hz, 1H), 6.80 (dd, J=8.6, 0.8 Hz, 1H), 5.25 (p, J=6.1 Hz, 1H), 2.37-2.27 (m, 2H), 2.17-2.08 (m, 1H), 1.94 (dd, J=14.9, 7.7 Hz, 1H), 1.30 (d, J=6.2 Hz, 6H), 0.98 (d, J=6.3 Hz, 2H), 0.92-0.87 (m, 5H).

m/z 419.2 (M+H)⁺

### 4-(6′-((4-isopropylphenyl)ethynyl)-1′H-spiro[cyclopropane-1,4′-isoquinolin]-2′(3′H)-yl)-3-methylbutanoic acid (Compound 60)

¹H NMR (400 MHz, DMSO-d₆) δ 7.46 (d, J=8.2 Hz, 2H), 7.30 (d, J=8.2 Hz, 2H), 7.24 (dd, J=7.8, 1.6 Hz, 1H), 7.11 (d, J=7.9 Hz, 1H), 6.85 (d, J=1.6 Hz, 1H), 2.90 (dq, J=14.2, 6.9 Hz, 2H), 2.37-2.28 (m, 4H), 2.18-2.08 (m, 1H), 1.95 (dd, J=15.2, 7.5 Hz, 1H), 1.23-1.19 (m, 10H), 1.00 (s, 2H), 0.90 (d, J=6.6 Hz, 5H).

m/z 402.2 (M+H)⁺

### 4-(6′-((3-fluoro-4-(trifluoromethoxy)phenyl)ethynyl)-1′H-spiro[cyclopropane-1,4′-isoquinolin]-2′(3′H)-yl)-3-methylbutanoic acid (Compound 61)

¹H NMR (400 MHz, DMSO-d₆) δ 7.72 (dd, J=11.0, 2.0 Hz, 1H), 7.60 (t, J=8.1 Hz, 1H), 7.50 (dt, J=8.5, 1.5 Hz, 1H), 7.28 (dd, J=7.8, 1.6 Hz, 1H), 7.14 (d, J=8.0 Hz, 1H), 6.92 (d, J=1.6 Hz, 1H), 2.29 (dt, J=15.4, 9.1 Hz, 3H), 2.18-2.08 (m, 1H), 1.96-1.88 (m, 1H), 1.00 (s, 2H), 0.93 (d, J=3.0 Hz, 1H), 0.90 (d, J=6.6 Hz, 4H).

m/z 462.2 (M+H)⁺

### 4-(6′-((4-isopropoxyphenyl)ethynyl)-1′H-spiro[cyclopropane-1,4′-isoquinolin]-2′(3′H)-yl)-3-methylbutanoic acid (Compound 62)

¹H NMR (400 MHz, DMSO-d₆) δ 7.47-7.43 (m, 2H), 7.22 (dd, J=7.8, 1.6 Hz, 1H), 7.10 (d, J=7.9 Hz, 1H), 6.97-6.92 (m, 3H), 6.83 (d, J=1.9 Hz, 1H), 4.70-4.55 (m, 1H), 3.69 (d, J=3.9 Hz, 1H), 2.57 (d, J=2.7 Hz, 2H), 2.35 (ddd, J=13.9, 9.3, 5.7 Hz, 4H), 2.19-2.09 (m, 1H), 1.99 (dd, J=15.1, 7.5 Hz, 1H), 1.28 (d, J=6.0 Hz, 6H), 1.01-0.98 (m, 2H), 0.91 (d, J=6.4 Hz, 6H).

m/z 418.2 (M+H)⁺

### 4-(6′-((4-ethoxyphenyl)ethynyl)-1′H-spiro[cyclopropane-1,4′-isoquinolin]-2′(3′H)-yl)-3-methylbutanoic acid (Compound 63)

¹H NMR (400 MHz, DMSO-d₆) δ 7.48-7.44 (m, 2H), 7.22 (dd, J=7.9, 1.6 Hz, 1H), 7.10 (d, J=8.0 Hz, 1H), 6.98-6.94 (m, 2H), 6.84 (d, J=1.7 Hz, 1H), 4.06 (q, J=7.0 Hz, 2H), 2.40-2.30 (m, 3H), 2.14 (dq, J=12.6, 6.3, 5.7 Hz, 1H), 2.00 (dd, J=15.1, 7.5 Hz, 1H), 1.34 (t, J=7.0 Hz, 4H), 1.00 (t, J=3.8 Hz, 2H), 0.91 (d, J=6.6 Hz, 5H).

m/z 404.2 (M+H)⁺

### 4-(6′-((4-(cyclopentyloxy)phenyl)ethynyl)-1′H-spiro[cyclopropane-1,4′-isoquinolin]-2′(3′H)-yl)-3-methylbutanoic acid (Compound 64)

¹H NMR (400 MHz, DMSO-d₆) δ 7.45 (d, J=8.8 Hz, 2H), 7.21 (d, J=7.8 Hz, 1H), 7.09 (d, J=8.0 Hz, 1H), 6.93 (d, J=8.8 Hz, 2H), 6.82 (s, 1H), 4.90-4.82 (m, OH), 2.24 (d, J=10.0 Hz, 2H), 1.94 (s, 6H), 1.88-1.81 (m, 1H), 1.70 (s, 1H), 1.60 (s, 7H), 0.99-0.93 (m, 2H), 0.88 (d, J=6.6 Hz, 3H), 0.86-0.80 (m, 2H).

m/z 444.2 (M+H)⁺

### 3-methyl-4-(6′-((4-(trifluoromethoxy)phenyl)ethynyl)-1′H-spiro[cyclopropane-1,4′-isoquinolin]-2′(3′H)-yl)butanoic acid (Compound 65)

¹H NMR (400 MHz, DMSO-d₆) δ 7.70-7.65 (m, 2H), 7.44-7.39 (m, 2H), 7.27 (dd, J=7.8, 1.6 Hz, 1H), 7.13 (d, J=8.0 Hz, 1H), 6.90 (d, J=1.5 Hz, 1H), 2.55 (s, 3H), 2.37-2.28 (m, 3H), 2.19-2.08 (m, 1H), 1.95 (dd, J=15.0, 7.6 Hz, 1H), 1.00 (s, 2H), 0.90 (d, J=6.5 Hz, 5H).

m/z 444.2 (M+H)⁺

### 4-(6′-((2,3-difluorophenyl)ethynyl)-1′H-spiro[cyclopropane-1,4′-isoquinolin]-2′(3′H)-yl)-3-methylbutanoic acid (Compound 66)

¹H NMR (400 MHz, Pyridine-d₅) δ 7.46-7.41 (m, 1H), 7.38 (tt, J=6.0, 1.6 Hz, 1H), 7.17 (d, J=1.6 Hz, 1H), 7.07 (dd, J=7.9, 2.3 Hz, 2H), 6.96 (d, J=7.9 Hz, 1H), 6.81-6.73 (m, 1H), 3.82-3.65 (m, 2H), 2.87 (ddd, J=15.1, 5.0, 1.9 Hz, 1H), 2.57-2.45 (m, 3H), 2.43-2.26 (m, 3H), 1.14 (dd, J=6.5, 3.5 Hz, 3H), 1.02-0.97 (m, 1H), 0.95-0.78 (m, 2H).

m/z 396.3 (M+H)⁺

### 3-methyl-4-(6′-(thiophen-3-ylethynyl)-1′H-spiro[cyclopropane-1,4′-isoquinolin]-2′(3′H)-yl)butanoic acid (Compound 68)

¹H NMR (400 MHz, Pyridine-d₅) δ 7.85 (dd, J=3.0, 1.2 Hz, 1H), 7.48 (dd, J=5.0, 3.0 Hz, 1H), 7.40 (dd, J=7.8, 1.6 Hz, 1H), 7.37 (dd, J=5.0, 1.2 Hz, 1H), 7.13 (d, J=1.5 Hz, 1H), 7.04 (d, J=7.9 Hz, 1H), 3.82-3.67 (m, 2H), 2.87 (dd, J=15.2, 4.9 Hz, 1H), 2.58-2.47 (m, 3H), 2.41-2.27 (m, 3H), 1.15 (d, J=6.5 Hz, 3H), 1.04-0.97 (m, 2H), 0.96-0.86 (m, 2H).

m/z 366.2 (M+H)⁺

### 4-(6′-((4-ethoxy-3-fluorophenyl)ethynyl)-1′H-spiro[cyclopropane-1,4′-isoquinolin]-2′(3′H)-yl)-3-methylbutanoic acid (Compound 69)

¹H NMR (400 MHz, DMSO-d₆) δ 7.40 (dd, J=11.9, 2.1 Hz, 1H), 7.33 (dd, J=8.5, 1.6 Hz, 1H), 7.23 (dd, J=7.8, 1.6 Hz, 1H), 7.18 (t, J=8.7 Hz, 1H), 7.11 (d, J=8.0 Hz, 1H), 6.86 (d, J=1.6 Hz, 1H), 4.14 (q, J=6.8 Hz, 2H), 2.56 (s, 2H), 2.40-2.29 (m, 2H), 2.14 (d, J=6.9 Hz, 1H), 1.99 (dd, J=15.0, 7.5 Hz, 1H), 1.36 (td, J=7.0, 2.6 Hz, 4H), 1.32-1.08 (m, 1H), 1.07-0.95 (m, 1H), 0.91 (d, J=6.4 Hz, 3H).

m/z 422.2 (M+H)⁺

### 4-(6′-((6-ethoxy-4-methylpyridin-3-yl)ethynyl)-1′H-spiro[cyclopropane-1,4′-isoquinolin]-2′(3′H)-yl)-3-methylbutanoic acid (Compound 67)

A microwave vial was charged with K₃PO₄ (69.54 mg, 3 eq, 0.33 mmol), XPHOS (12.52 mg, 24 mol %, 0.03 mmol), PdCl₂(MeCN)₂ (4.25 mg, 15 mol %, 0.02 mmol), and stir bar. This was capped with a septa crimp cap and placed under N₂ to purge for a couple of minutes. To this, via syringe, 500 μL of a solution of ethyl 4-(6′-bromo-1′H-spiro[cyclopropane-1,4′-isoquinolin]-2′(3′H)-yl)-3-methylbutanoate in dry Acetonitrile (40 mg, 0.11 mmol) and 365 μL of a solution 2-ethoxy-5-ethynyl-4-methylpyridine that was prepared from a pre-weighed vial containing 0.6 mmol of substrate dissolved in 1000 μL of dry Acetonitrile (35.21 mg, 1.5 eq, 0.09 mmol) was added. The vial was left to purge for a couple of minutes more under N₂ and then placed to heat at 100° C. for 18 hours until reaction is complete. Upon completion the compound was filtered using a Celite cartridge washing 2 times with MeOH. This is then dried under a N₂ blower. The dried crude material is then dissolved in 500 μL of THF and 1500 μL of an aqueous solution of 1M LiOH in 75% MeOH. This was capped and left to stir at 60° C. for 2 hour. Upon completion the crude material was filtered once more in a celite cartridge and washed 2 times with 1000 μL of Acetonitrile. The crude material is dried once more under N2 blower and then dissolved in 1400 μL of DMSO/Acetonitrile. The crude product was purified by reverse phase HPLC using the AA method described above to yield 4-(6′-((6-ethoxy-4-methylpyridin-3-yl)ethynyl)-1′H-spiro[cyclopropane-1,4′-isoquinolin]-2′(3′H)-yl)-3-methylbutanoic acid (16.7 mg, 38.51%).

¹H NMR (400 MHz, Pyridine-d₅) δ 9.98 (s, 1H), 8.89 (dd, J=7.8, 1.6 Hz, 1H), 8.62 (d, J=1.5 Hz, 1H), 8.53 (d, J=7.9 Hz, 1H), 8.15 (s, 1H), 5.86 (q, J=7.0 Hz, 2H), 5.28-5.16 (m, 2H), 4.32 (dd, J=15.1, 4.9 Hz, 1H), 4.03-3.94 (m, 3H), 3.89-3.83 (m, 1H), 3.82 (s, 3H), 3.79 (dd, J=7.4, 2.2 Hz, 1H), 2.74 (t, J=7.1 Hz, 3H), 2.59 (d, J=6.4 Hz, 3H), 2.54-2.43 (m, 2H), 2.36 (qd, J=8.9, 4.3 Hz, 2H).

m/z 419.3 (M+H)⁺

### 3-(6′-((5-isopropoxypyridin-2-yl)ethynyl)-1′H-spiro[cyclopropane-1,4′-isoquinolin]-2′(3′H)-yl)cyclobutanecarboxylic acid (compound 70)

A microwave vial was charged with K₃PO₄ (55.8 mg, 3 eq, 0.26 mmol), XPHOS (10.04 mg, 24 mol %, 0.13 mmol), PdCl₂(MeCN)₂ (3.4 mg, 15 mol %, 0.01 mmol), and stir bar. This was capped with a septa crimp cap and placed under N₂ to purge for a couple of minutes. To this, via syringe, 500 μL of a solution of methyl 3-(6′-bromo-1′H-spiro[cyclopropane-1,4′-isoquinolin]-2′(3′H)-yl)cyclobutanecarboxylate in dry Acetonitrile (30.7 mg, 0.09 mmol) and 219 μL of a solution 2-ethoxy-5-ethynyl-3-fluoropyridine that was prepared from a pre-weighed vial containing 0.6 mmol of substrate dissolved in 1000 μL of dry Acetonitrile (21.7 mg, 1.5 eq, 0.2 mmol) was added. The vial was left to purge for a couple of minutes more under N₂ and then placed to heat at 100° C. for 18 hours until reaction is complete. Upon completion the compound was filtered using a Celite cartridge washing 2 times with MeOH. This is then dried under a N₂ blower. The dried crude material is then dissolved in 500 μL of THF and 1500 μL of an aqueous solution of 1M LiOH in 75% MeOH. This was capped and left to stir at 60° C. for 2 hour. Upon completion the crude material was filtered once more in a celite cartridge and washed 2 times with 1000 μL of Acetonitrile. The crude material is dried once more under N₂ blower and then dissolved in 1400 μL of DMSO/Acetonitrile. The crude product was purified by reverse phase HPLC using the AA method described above to yield 3-(6′-((6-ethoxy-5-fluoropyridin-3-yl)ethynyl)-1′H-spiro[cyclopropane-1,4′-isoquinolin]-2′(3′H)-yl)cyclobutanecarboxylic acid (2.4 mg, 6.8%).

¹H NMR (400 MHz, Pyridine-d₅) δ 8.59 (d, J=2.9 Hz, 1H), 7.65 (d, J=8.6 Hz, 1H), 7.44 (d, J=7.9 Hz, 1H), 7.29 (dd, J=8.6, 3.0 Hz, 1H), 7.19 (s, 1H), 7.07-6.98 (m, 1H), 4.56 (p, J=6.1 Hz, 1H), 3.62 (s, 2H), 3.34-3.24 (m, OH), 3.07 (t, J=9.0 Hz, 1H), 2.80 (t, J=7.7 Hz, 1H), 2.67 (d, J=5.6 Hz, 1H), 2.47 (t, J=8.5 Hz, 3H), 2.39 (d, J=6.6 Hz, 2H), 1.23 (d, J=6.0 Hz, 6H), 1.02 (t, J=5.8 Hz, 2H), 0.88 (dd, J=8.3, 4.0 Hz, 2H).

No MS

The following compounds were prepared in the same way as compound 70.

### 3-(6′-((3-fluoro-4-(trifluoromethoxy)phenyl)ethynyl)-1′H-spiro[cyclopropane-1,4′-isoquinolin]-2′(3′H)-yl)cyclobutanecarboxylic acid (Compound 71)

¹H NMR (400 MHz, Pyridine-d₅) δ 7.56 (d, J=1.4 Hz, 0H), 7.50-7.41 (m, 3H), 7.10 (d, J=7.9 Hz, 1H), 3.66 (s, 2H), 3.14-3.02 (m, 1H), 2.90-2.75 (m, 1H), 2.49 (t, J=8.5 Hz, 3H), 2.43 (s, 2H), 1.10-1.04 (m, 2H), 0.96-0.86 (m, 2H).

m/z 460.3 (M+H)⁺

### 3-(6′-((4-ethoxy-3-fluorophenyl)ethynyl)-1′H-spiro[cyclopropane-1,4′-isoquinolin]-2′(3′H)-yl)cyclobutanecarboxylic acid (compound 72)

¹H NMR (400 MHz, Pyridine-d₅) δ 7.56-7.40 (m, 2H), 7.11-6.99 (m, 1H), 3.97 (d, J=7.0 Hz, 1H), 3.65 (s, 2H), 2.52-2.46 (m, 6H), 2.42 (s, 1H), 1.31-1.25 (m, 8H), 1.09-1.03 (m, 1H), 0.91-0.87 (m, 2H).

m/z 420.3 (M+H)⁺

### 3-(6′-((5-ethoxypyridin-2-yl)ethynyl)-1′H-spiro[cyclopropane-1,4′-isoquinolin]-2′(3′H)-yl)cyclobutanecarboxylic acid (compound 73)

¹H NMR (400 MHz, Pyridine-d₅) δ 8.57 (d, J=2.9 Hz, 1H), 7.63 (d, J=8.7 Hz, 1H), 7.43 (d, J=7.7 Hz, 1H), 7.25 (dd, J=8.8, 3.0 Hz, 2H), 7.19 (d, J=1.5 Hz, 1H), 7.06-7.00 (m, 1H), 3.94 (q, J=7.0 Hz, 2H), 3.62 (s, 2H), 3.12-3.01 (m, 1H), 2.80 (p, J=7.5 Hz, 1H), 2.47 (t, J=8.5 Hz, 2H), 2.39 (d, J=6.6 Hz, 2H), 1.26 (t, J=7.0 Hz, 3H), 1.02 (d, J=5.9 Hz, 2H), 0.92-0.83 (m, 2H).

m/z 403.2 (M+H)⁺

### 1-((6′-((6-ethoxy-4-methylpyridin-3-yl)ethynyl)-1′H-spiro[cyclopropane-1,4′-isoquinolin]-2′(3′H)-yl)methyl)cyclopropanecarboxylic acid (Compound 74)

A microwave vial was charged with K₃PO₄ (39.7 mg, 3 eq, 0.20 mmol), XPHOS (7.14 mg, 24 mol %, 0.01 mmol), PdCl₂(MeCN)₂ (2.4 mg, 15 mol %, 0.01 mmol), and stir bar. This was capped with a septa crimp cap and placed under N₂ to purge for a couple of minutes. To this, via syringe, 500 μL of a solution of methyl 1-((6′-bromo-1′H-spiro[cyclopropane-1,4′-isoquinolin]-2′(3′H)-yl)methyl)cyclopropanecarboxylate in dry Acetonitrile (21.8 mg, 0.06 mmol) and 160 μL of a solution 2-ethoxy-5-ethynyl-4-methylpyridine that was prepared from a pre-weighed vial containing 0.6 mmol of substrate dissolved in 1000 μL of dry Acetonitrile (15.1 mg, 1.5 eq, 0.09 mmol) was added. The vial was left to purge for a couple of minutes more under N₂ and then placed to heat at 100° C. for 18 hours until reaction is complete. Upon completion the compound was filtered using a Celite cartridge washing 2 times with MeOH. This is then dried under a N₂ blower. The dried crude material is then dissolved in 500 μL of THF and 1500 μL of an aqueous solution of 1M LiOH in 75% MeOH. This was capped and left to stir at 60° C. for 2 hour. Upon completion the crude material was filtered once more in a celite cartridge and washed two times with 1000 μL of Acetonitrile. The crude material is dried once more under N₂ blower and then dissolved in 1400 μL of DMSO/Acetonitrile. The crude product was purified by reverse phase HPLC using the AA method described above to yield 1-((6′-((6-ethoxy-4-methylpyridin-3-yl)ethynyl)-1′H-spiro[cyclopropane-1,4′-isoquinolin]-2′(3′H)-yl)methyl)cyclopropanecarboxylic acid (2.4 mg, 9.6%). 1H NMR (400 MHz, Pyridine-d₅) δ 7.46 (dd, J=8.2, 1.3 Hz, 1H), 7.40-7.35 (m, 3H), 7.18 (d, J=1.4 Hz, 1H), 7.10 (d, J=7.9 Hz, 1H), 6.72 (s, 1H), 4.43 (q, J=7.0 Hz, 2H), 3.99 (s, 2H), 2.87 (s, 2H), 2.77 (s, 2H), 2.39 (s, 3H), 1.54-1.46 (m, 2H), 1.30 (t, J=7.0 Hz, 4H), 1.10-1.02 (m, 4H), 0.98-0.90 (m, 3H), 0.84 (q, J=3.3 Hz, 2H).

m/z 417.3 (M+H)⁺

### 3-(6-((2,3-difluorophenyl)ethynyl)-3,4-dihydroisoquinolin-2(1H)-yl)propanoic acid (Reference Compound 1)

Reference compound 1 was prepared in a similar way as the compounds of the present invention, except starting with a different intermediate than intermediate compound X (i.e. one lacking the spiro-linked group). The S1P5 EC50, hS1P5 Emax and S1P3 EC50 were determined as described below for the compounds of the invention.

S1P5 EC50=0.0290; hS1P5 EMax=60; S1P3 EC50>1 μM

### 3-(6-((2,6-dichlorophenyl)ethynyl)-3,4-dihydroisoquinolin-2 (1H)-yl)propanoic acid (Reference Compound 2)

Reference compound 2 was prepared in a similar way as the compounds of the present invention, except starting with a different intermediate than intermediate compound X (i.e. one lacking the spiro-linked group). The S1P5 EC50, hS1P5 Emax and S1P3 EC50 were determined as described below for the compounds of the invention.

S1P5 EC50=0.0192; hS1P5 EMax=41; S1P3 EC50>1 μM

TABLE 1

| | | S1P5 | | | | |
|---|---|---|---|---|---|---|
| | | EC50 | | | | |
| | | range | hS1P5 | S1P1 | S1P3 | S1P4 |
| No | Structure | # | EMax | EC50* | EC50* | EC50* |
| 1 | 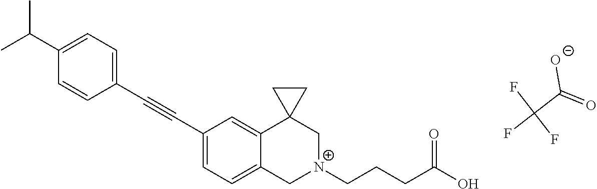 [SMILES: CC(C)c1ccc(C#Cc2ccc3c(c2)C2(CC2)C[NH+](CCCC(=O)O)C3)cc1 ; O=C([O-])C(F)(F)F] | A | ++ | C | >1 μM | >1 μM |
| 2 | 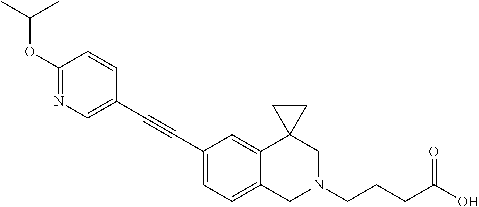 [SMILES: CC(C)Oc1ccc(C#Cc2ccc3c(c2)C2(CC2)CN(CCCC(=O)O)C3)cn1] | A | ++ | >1 μM | >1 μM | >1 μM |
| 3 | 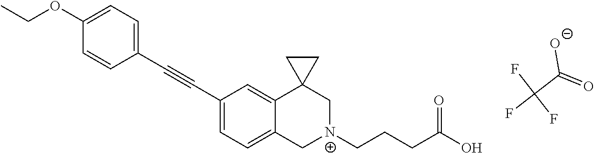 [SMILES: CCOc1ccc(C#Cc2ccc3c(c2)C2(CC2)C[NH+](CCCC(=O)O)C3)cc1 ; O=C([O-])C(F)(F)F] | A | ++ | C | >1 μM | >1 μM |
| 4 | 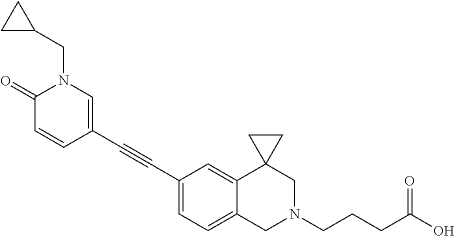 [SMILES: O=C(O)CCCN1Cc2ccc(C#Cc3ccc(=O)n(CC4CC4)c3)cc2C2(CC2)C1] | D | ++ | >1 μM | | >1 μM |
| 5 | 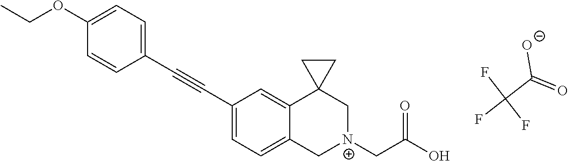 [SMILES: CCOc1ccc(C#Cc2ccc3c(c2)C2(CC2)C[NH+](CC(=O)O)C3)cc1 ; O=C([O-])C(F)(F)F] | C | ++ | >1 μM | | >1 μM |
| 6 | 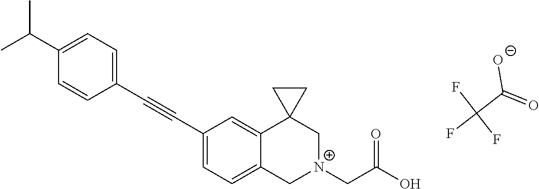 [SMILES: CC(C)c1ccc(C#Cc2ccc3c(c2)C2(CC2)C[NH+](CC(=O)O)C3)cc1 ; O=C([O-])C(F)(F)F] | C | +++ | >1 μM | | >1 μM |
| 8 | 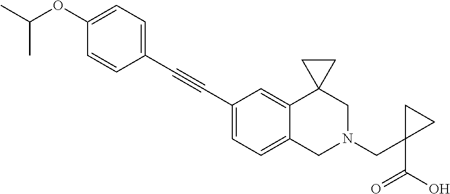 [SMILES: CC(C)Oc1ccc(C#Cc2ccc3c(c2)C2(CC2)CN(CC2(C(=O)O)CC2)C3)cc1] | C | ++ | >1 μM | | >1 μM |
| 9 | 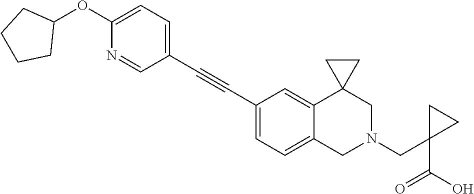 [SMILES: O=C(O)C1(CN2Cc3ccc(C#Cc4ccc(OC5CCCC5)nc4)cc3C3(CC3)C2)CC1] | C | +++ | >1 μM | | >1 μM |
| 10 | 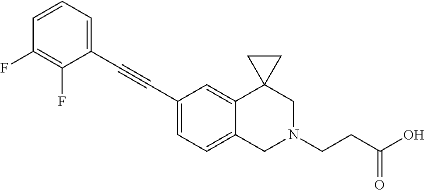 [SMILES: O=C(O)CCN1Cc2ccc(C#Cc3cccc(F)c3F)cc2C2(CC2)C1] | A | +++ | >1 μM | >1 μM | B |
| 11 | 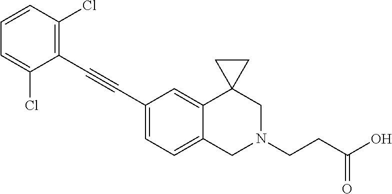 [SMILES: O=C(O)CCN1Cc2ccc(C#Cc3c(Cl)cccc3Cl)cc2C2(CC2)C1] | A | ++ | >1 μM | >1 μM | B |
| 12 | 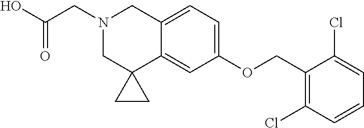 [SMILES: O=C(O)CN1Cc2ccc(OCc3c(Cl)cccc3Cl)cc2C2(CC2)C1] | C | ++ | >1 μM | | C |
| 13 | 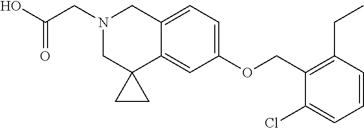 [SMILES: CCc1cccc(Cl)c1COc1ccc2c(c1)C1(CC1)CN(CC(=O)O)C2] | A | ++ | >1 μM | >1 μM | C |
| 14 | 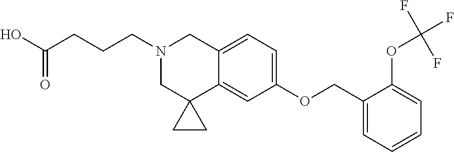 [SMILES: O=C(O)CCCN1Cc2ccc(OCc3ccccc3OC(F)(F)F)cc2C2(CC2)C1] | A | ++ | >1 μM | >1 μM | C |
| 15 | 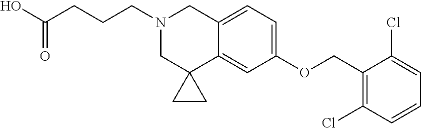 [SMILES: O=C(O)CCCN1Cc2ccc(OCc3c(Cl)cccc3Cl)cc2C2(CC2)C1] | A | +++ | >1 μM | >1 μM | B |
| 16 | 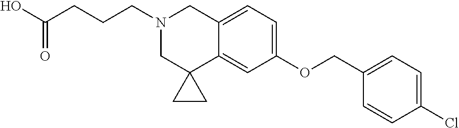 [SMILES: O=C(O)CCCN1Cc2ccc(OCc3ccc(Cl)cc3)cc2C2(CC2)C1] | C | + | >1 μM | | C |
| 17 | 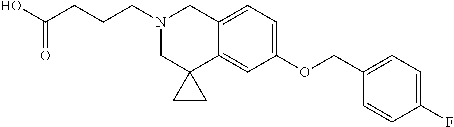 [SMILES: O=C(O)CCCN1Cc2ccc(OCc3ccc(F)cc3)cc2C2(CC2)C1] | C | ++ | >1 μM | | >1 μM |
| 18 | 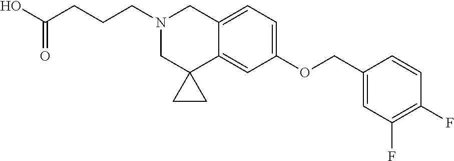 [SMILES: O=C(O)CCCN1Cc2ccc(OCc3ccc(F)c(F)c3)cc2C2(CC2)C1] | B | + | >1 μM | >1 μM | C |
| 19 | 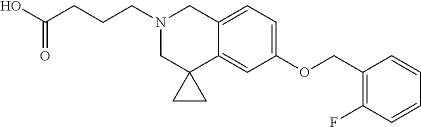 [SMILES: O=C(O)CCCN1Cc2ccc(OCc3ccccc3F)cc2C2(CC2)C1] | A | ++ | >1 μM | >1 μM | >1 μM |
| 20 | 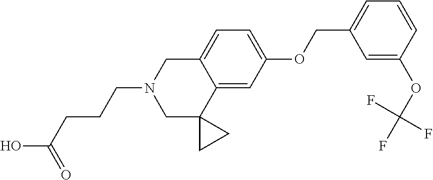 [SMILES: O=C(O)CCCN1Cc2ccc(OCc3cccc(OC(F)(F)F)c3)cc2C2(CC2)C1] | B | + | >1 μM | >1 μM | C |
| 21 | 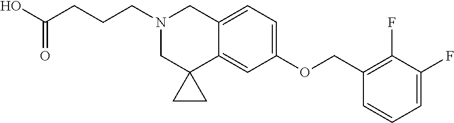 [SMILES: O=C(O)CCCN1Cc2ccc(OCc3cccc(F)c3F)cc2C2(CC2)C1] | B | + | >1 μM | >1 μM | C |
| 22 | 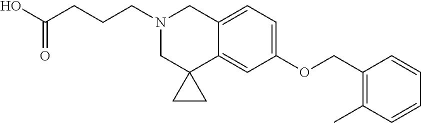 [SMILES: Cc1ccccc1COc1ccc2c(c1)C1(CC1)CN(CCCC(=O)O)C2] | A | ++ | >1 μM | >1 μM | C |
| 23 | 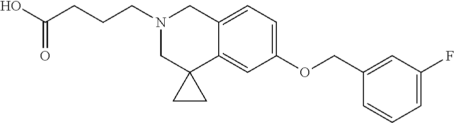 [SMILES: O=C(O)CCCN1Cc2ccc(OCc3cccc(F)c3)cc2C2(CC2)C1] | B | ++ | >1 μM | >1 μM | >1 μM |
| 24 | 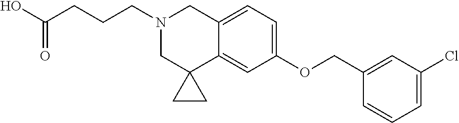 [SMILES: O=C(O)CCCN1Cc2ccc(OCc3cccc(Cl)c3)cc2C2(CC2)C1] | A | ++ | >1 μM | >1 μM | C |
| 25 | 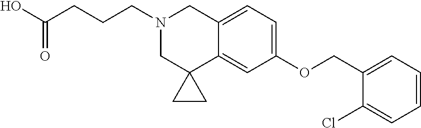 [SMILES: O=C(O)CCCN1Cc2ccc(OCc3ccccc3Cl)cc2C2(CC2)C1] | A | ++ | >1 μM | >1 μM | C |
| 26 | 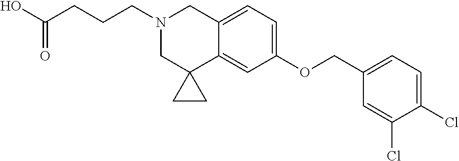 [SMILES: O=C(O)CCCN1Cc2ccc(OCc3ccc(Cl)c(Cl)c3)cc2C2(CC2)C1] | B | ++ | >1 μM | >1 μM | B |
| 27 | 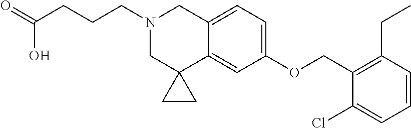 [SMILES: CCc1cccc(Cl)c1COc1ccc2c(c1)C1(CC1)CN(CCCC(=O)O)C2] | A | +++ | >1 μM | >1 μM | A |
| 28 | 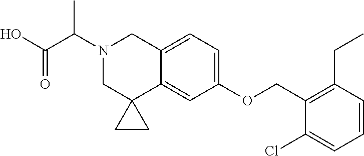 [SMILES: CCc1cccc(Cl)c1COc1ccc2c(c1)C1(CC1)CN(C(C)C(=O)O)C2] | B | ++ | >1 μM | >1 μM | C |
| 29 | 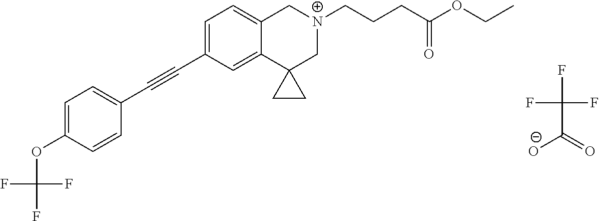 [SMILES: CCOC(=O)CCC[NH+]1Cc2ccc(C#Cc3ccc(OC(F)(F)F)cc3)cc2C2(CC2)C1 ; O=C([O-])C(F)(F)F] | | | | | |
| 30 | 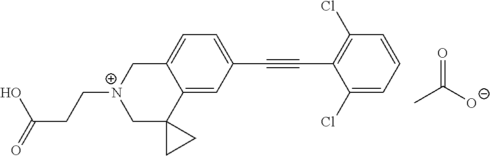 | A | ++ | >1 μM | >1 μM | C |
| 31 | 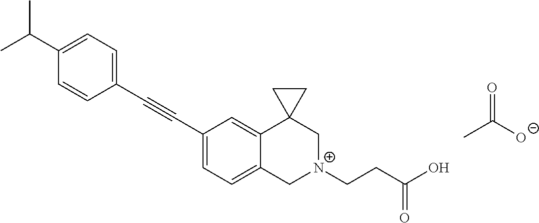 | A | +++ | C | >1 μM | >1 μM |
| 32 | 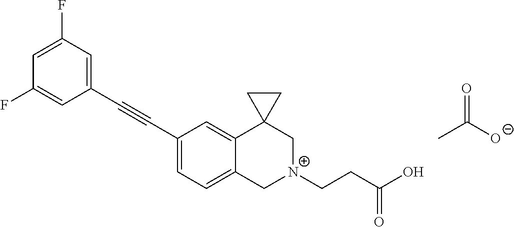 | A | ++ | >1 μM | >1 μM | C |
| 33 | 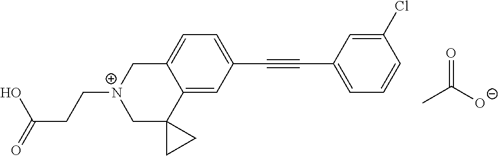 | A | + | >1 μM | >1 μM | A |
| 34 | 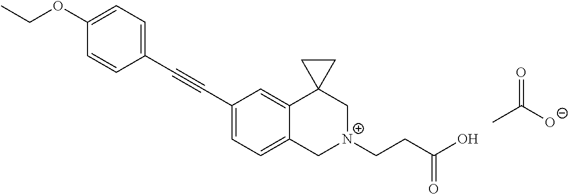 | A | ++ | >1 μM | >1 μM | >1 μM |
| 35 | 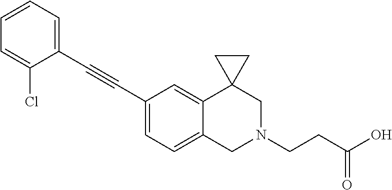 [SMILES: O=C(O)CCN1Cc2ccc(C#Cc3ccccc3Cl)cc2C2(CC2)C1] | A | ++ | >1 μM | >1 μM | C |
| 36 | 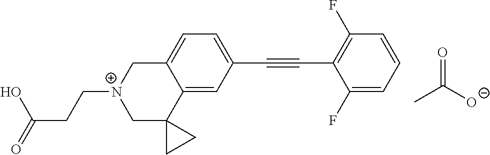 | A | ++ | >1 μM | >1 μM | C |
| 37 | 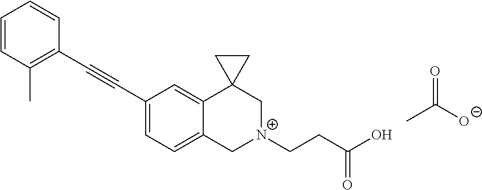 | B | ++ | >1 μM | >1 μM | C |
| 38 | 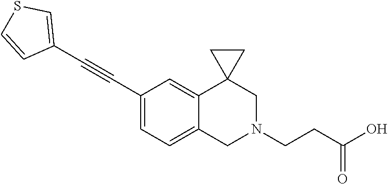 [SMILES: O=C(O)CCN1Cc2ccc(C#Cc3ccsc3)cc2C2(CC2)C1] | B | ++ | >1 μM | >1 μM | >1 μM |
| 39 | 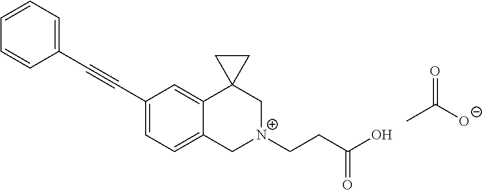 | C | ++ | >1 μM | >1 μM | >1 μM |
| 40 | 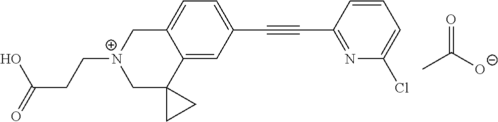 | C | ++ | >1 μM | >1 μM | C |
| 41 | 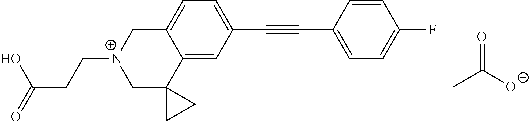 | D | + | >1 μM | >1 μM | >1 μM |
| 42 | 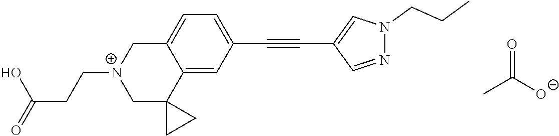 | D | ++ | >1 μM | >1 μM | >1 μM |
| 43 | 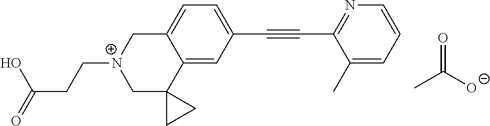 | D | ++ | >1 μM | >1 μM | >1 μM |
| 44 | 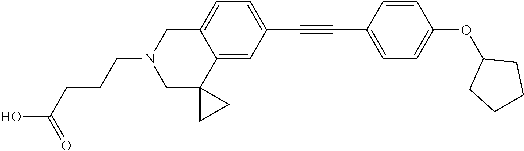 [SMILES: O=C(O)CCCN1Cc2ccc(C#Cc3ccc(OC4CCCC4)cc3)cc2C2(CC2)C1] | A | +++ | C | >1 μM | >1 μM |
| 45 | 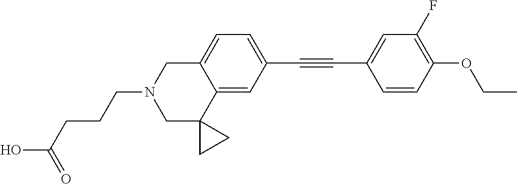 [SMILES: CCOc1ccc(C#Cc2ccc3c(c2)C2(CC2)CN(CCCC(=O)O)C3)cc1F] | A | ++ | >1 μM | >1 μM | >1 μM |
| 46 | 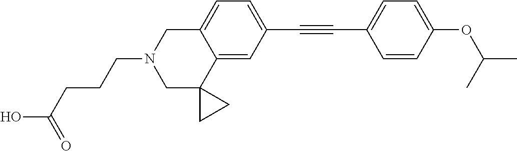 [SMILES: CC(C)Oc1ccc(C#Cc2ccc3c(c2)C2(CC2)CN(CCCC(=O)O)C3)cc1] | A | +++ | >1 μM | >1 μM | >1 μM |
| 47 | 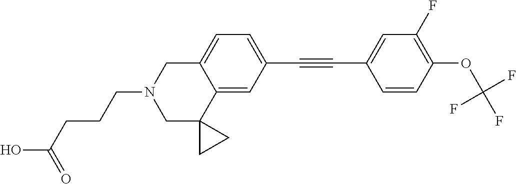 [SMILES: O=C(O)CCCN1Cc2ccc(C#Cc3ccc(OC(F)(F)F)c(F)c3)cc2C2(CC2)C1] | A | +++ | >1 μM | >1 μM | >1 μM |
| 48 | 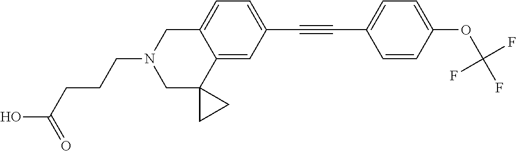 [SMILES: O=C(O)CCCN1Cc2ccc(C#Cc3ccc(OC(F)(F)F)cc3)cc2C2(CC2)C1] | B | ++ | >1 μM | >1 μM | >1 μM |
| 49 | 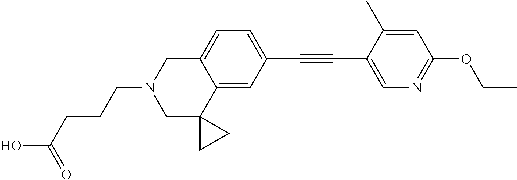 [SMILES: CCOc1cc(C)c(C#Cc2ccc3c(c2)C2(CC2)CN(CCCC(=O)O)C3)cn1] | B | ++ | >1 μM | | >1 μM |
| 50 | 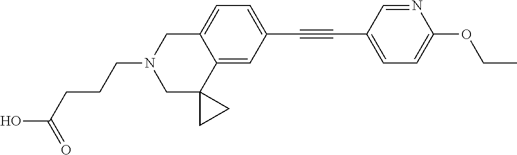 [SMILES: CCOc1ccc(C#Cc2ccc3c(c2)C2(CC2)CN(CCCC(=O)O)C3)cn1] | C | ++ | >1 μM | | >1 μM |
| 51 | 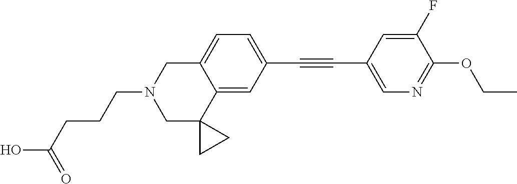 [SMILES: CCOc1ncc(C#Cc2ccc3c(c2)C2(CC2)CN(CCCC(=O)O)C3)cc1F] | C | ++ | >1 μM | | >1 μM |
| 52 | 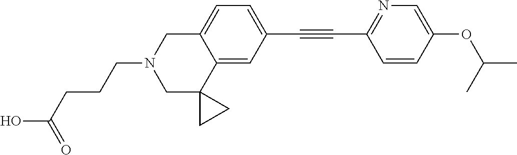 [SMILES: CC(C)Oc1ccc(C#Cc2ccc3c(c2)C2(CC2)CN(CCCC(=O)O)C3)nc1] | C | +++ | >1 μM | | >1 μM |
| 53 | 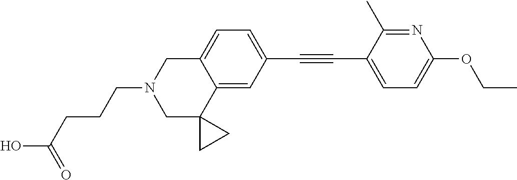 [SMILES: CCOc1ccc(C#Cc2ccc3c(c2)C2(CC2)CN(CCCC(=O)O)C3)c(C)n1] | C | ++ | >1 μM | | >1 μM |
| 54 | 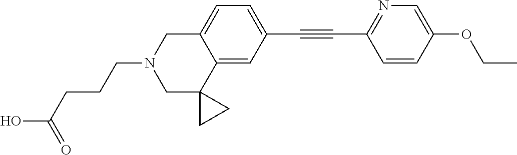 [SMILES: CCOc1ccc(C#Cc2ccc3c(c2)C2(CC2)CN(CCCC(=O)O)C3)nc1] | D | ++ | >1 μM | | >1 μM |
| 55 | 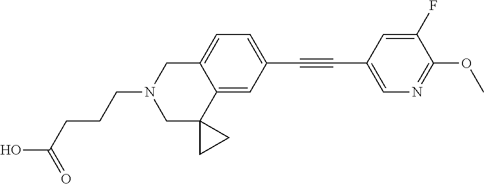 [SMILES: COc1ncc(C#Cc2ccc3c(c2)C2(CC2)CN(CCCC(=O)O)C3)cc1F] | D | + | >1 μM | | >1 μM |
| 56 | 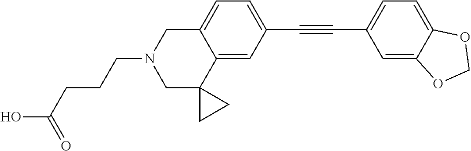 [SMILES: O=C(O)CCCN1Cc2ccc(C#Cc3ccc4c(c3)OCO4)cc2C2(CC2)C1] | D | ++ | >1 μM | | >1 μM |
| 57 | 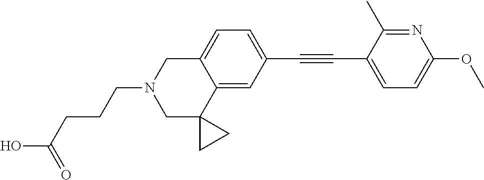 [SMILES: COc1ccc(C#Cc2ccc3c(c2)C2(CC2)CN(CCCC(=O)O)C3)c(C)n1] | D | ++ | >1 μM | | >1 μM |
| 58 | 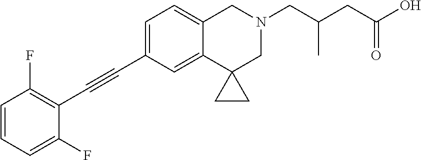 [SMILES: CC(CC(=O)O)CN1Cc2ccc(C#Cc3c(F)cccc3F)cc2C2(CC2)C1] | A | ++ | >1 μM | >1 μM | C |
| 59 | 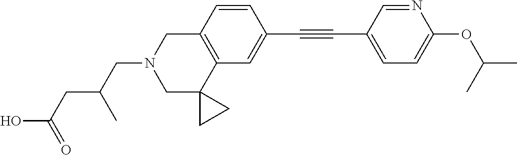 [SMILES: CC(CC(=O)O)CN1Cc2ccc(C#Cc3ccc(OC(C)C)nc3)cc2C2(CC2)C1] | A | ++ | >1 μM | | >1 μM |
| 60 | 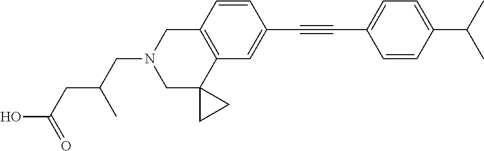 [SMILES: CC(CC(=O)O)CN1Cc2ccc(C#Cc3ccc(C(C)C)cc3)cc2C2(CC2)C1] | A | ++ | >1 μM | | >1 μM |
| 61 | 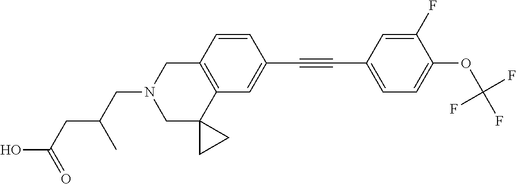 [SMILES: CC(CC(=O)O)CN1Cc2ccc(C#Cc3ccc(OC(F)(F)F)c(F)c3)cc2C2(CC2)C1] | A | ++ | >1 μM | | >1 μM |
| 62 | 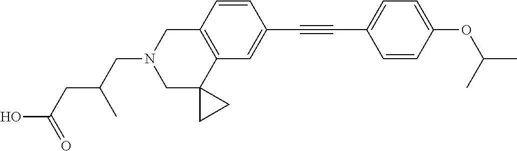 [SMILES: CC(CC(=O)O)CN1Cc2ccc(C#Cc3ccc(OC(C)C)cc3)cc2C2(CC2)C1] | A | +++ | >1 μM | | >1 μM |
| 63 | 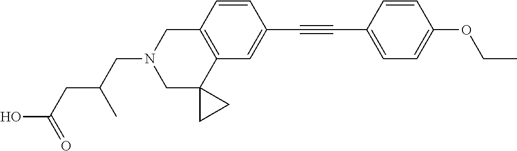 [SMILES: CCOc1ccc(C#Cc2ccc3c(c2)C2(CC2)CN(CC(C)CC(=O)O)C3)cc1] | B | ++ | >1 μM | | >1 μM |
| 64 | 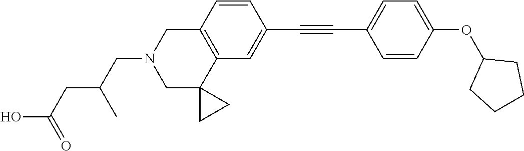 [SMILES: CC(CC(=O)O)CN1Cc2ccc(C#Cc3ccc(OC4CCCC4)cc3)cc2C2(CC2)C1] | B | +++ | >1 μM | | >1 μM |
| 65 | 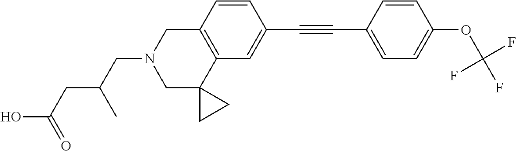 [SMILES: CC(CC(=O)O)CN1Cc2ccc(C#Cc3ccc(OC(F)(F)F)cc3)cc2C2(CC2)C1] | C | +++ | >1 μM | | >1 μM |
| 66 | 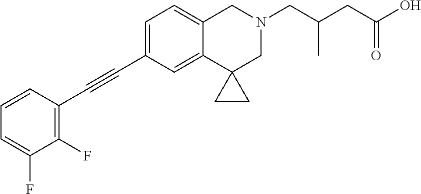 [SMILES: CC(CC(=O)O)CN1Cc2ccc(C#Cc3cccc(F)c3F)cc2C2(CC2)C1] | C | ++ | >1 μM | | >1 μM |
| 67 | 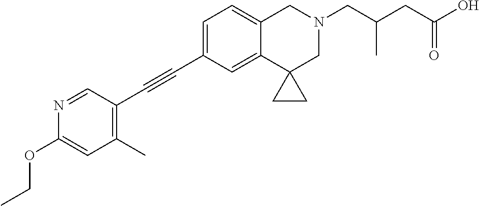 [SMILES: CCOc1cc(C)c(C#Cc2ccc3c(c2)C2(CC2)CN(CC(C)CC(=O)O)C3)cn1] | B | ++ | >1 μM | | >1 μM |
| 68 | 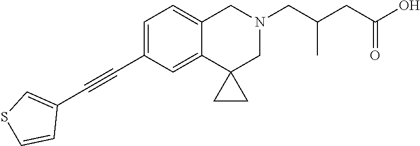 [SMILES: CC(CC(=O)O)CN1Cc2ccc(C#Cc3ccsc3)cc2C2(CC2)C1] | B | ++ | >1 μM | | >1 μM |
| 69 | 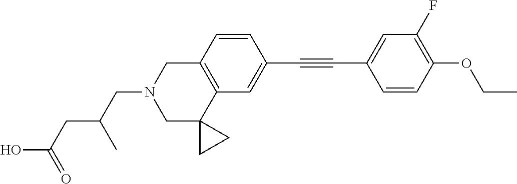 [SMILES: CCOc1ccc(C#Cc2ccc3c(c2)C2(CC2)CN(CC(C)CC(=O)O)C3)cc1F] | B | +++ | >1 μM | | >1 μM |
| 70 | 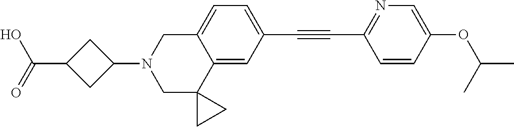 [SMILES: CC(C)Oc1ccc(C#Cc2ccc3c(c2)C2(CC2)CN(C2CC(C(=O)O)C2)C3)nc1] | B | ++ | >1 μM | | >1 μM |
| 71 | 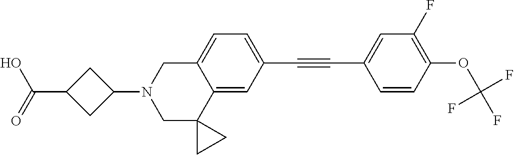 [SMILES: O=C(O)C1CC(N2Cc3ccc(C#Cc4ccc(OC(F)(F)F)c(F)c4)cc3C3(CC3)C2)C1] | B | ++ | >1 μM | | >1 μM |
| 72 | 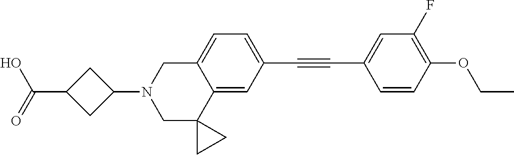 [SMILES: CCOc1ccc(C#Cc2ccc3c(c2)C2(CC2)CN(C2CC(C(=O)O)C2)C3)cc1F] | B | ++ | >1 μM | | >1 μM |
| 73 | 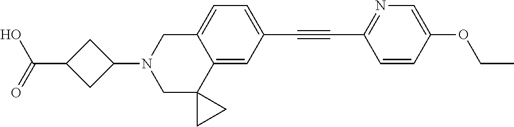 [SMILES: CCOc1ccc(C#Cc2ccc3c(c2)C2(CC2)CN(C2CC(C(=O)O)C2)C3)nc1] | D | ++ | >1 μM | | >1 μM |
| 74 | 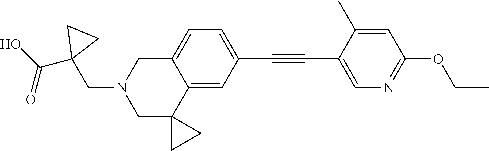 [SMILES: CCOc1cc(C)c(C#Cc2ccc3c(c2)C2(CC2)CN(CC2(C(=O)O)CC2)C3)cn1] | | | | | |

#A: EC50 <10 nM, determined with method 1
B: EC50 = 10 nM-100 nM, determined with method 1
C: EC50 = 100 nM-1 μM, determined with method 1
D: EC50 = 1 μM-10 μM, determined with method 1
+++: EMax >100, determined with method 1
++: EMax = 50-100, determined with method 1
+: EMax <50, determined with method 1

### 5. Assessment of Potency and Efficacy of Agonists

Agonistic activity and efficacy of compounds was assessed in recombinant cells using method 1 described below. The results are shown in table 1.

### Method 1. Intracellular Ca²⁺ Release

Agonist potency and efficacy were assessed by measurement of intracellular Ca²⁺ release. Recombinant CHO-K1 cells (Euroscreen, Brussels, Belgium) expressing human S1PR5, S1PR1, S1PR3 or S1PR4 receptors, aequorin, and GTP binding protein Gq/i5 were cultured using a medium containing nutrient mixture F-12 Ham (Sigma-Aldrich) with 10% FBS, and 100 μg/mL gentamicin and equilibrated at 5% CO₂.

15,000 cells in 20 μL medium were seeded into Biocoat poly-D-Lysine coated 384 well plates (Becton Dickinson #35-6663) and grown to 95% confluency after 24 h.

Culture medium was replaced by an assay buffer consisting of HBBS with Ca²⁺ and Mg²⁺ (Invitrogen #14025-050), 20 mmol/L Hepes (Sigma-Aldrich #H-3375), 2.5 mmol/L probenecid (Sigma-Aldrich #P-8761, and 0.1% BSA (Sigma-Aldrich #A-7030) pH 7.4. The Calcium 5 no-wash FLIPR assay kit (Molecular Devices #5000625) was performed as described in the kit instructions. Cells were incubated with Calcium 5 dye for 1 h at 37° C., 5% CO₂ in the dark. After 45 min adaptation to RT assessment of agonist stimulation of intracellular Ca²⁺ release was performed by addition of test compounds at various concentrations obtained by serial dilution. Phospho-fingolimod was used as positive control and reference agonist (intrinsic activity 100%).

Agonism of test compounds at the human S1Px receptors were characterized by the EC₅₀ values (potencies) deduced from a nonlinear 4 parametric logistic curve fit to the measured fluorescence data after normalization to the lower and upper plateaus of the fitted reference agonist's (phospho-fingolimod)effect curve and by their efficacy (Emax) values represented by their maximum achievable effects in % of the reference agonist's efficacy, which is defined as an Emax of 100%.

1. A compound of formula (I): 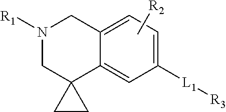
or a pharmaceutically acceptable salt thereof, wherein
R1 is selected from the group consisting of
—(C1-6)alkylene-R4 wherein one or more carbon atoms in the alkylene group, each independently, are optionally substituted with (CH₂)₂ to form a cyclopropyl moiety or with (CH₂)₃ to form a cyclobutyl moiety, —(C3-6)cycloalkylene-R4
—(C1-3)alkylene-(C3-6)cycloalkylene-R4, —(C3-6)cycloalkylene-(C1-3)alkylene-R4, and
—(C1-3)alkylene-(C3-6)cycloalkylene-(C1-3)alkylene-R4,
wherein one or more carbon atoms in the alkylene and cycloalkylene groups, each independently, are optionally substituted with one or more halogen atoms; and
wherein R4 is selected from the group consisting of —COOH and —COO(C1-4)alkyl;
R2 is hydrogen, or R2 is one or more substituents independently selected from the group consisting of a halogen atom, (C1-4)alkyl optionally substituted with one or more halogen atoms, (C3-4)cycloalkyl optionally substituted with one or more halogen atoms, (C1-4)alkoxy optionally substituted with one or more halogen atoms, (C3-4)cycloalkoxy optionally substituted with one or more halogen atoms, and hydroxyl;
L1 is selected from the group consisting of —C≡C—, —O—CH₂—, —CH₂—O—, —CH₂—CH₂—, —CH═CH—, —CF₂—CH₂—, —CH₂—CF₂—, —CHF—CH₂—, —CH₂—CHF—, —S—CH₂—, —CH₂—S—, —O— and —S—,
R3 is a 5- to 6-membered ring selected from the group consisting of unsaturated 6-membered rings, unsaturated 5-membered rings, saturated 6-membered rings and saturated 5-membered rings,
wherein the 5- to 6-membered ring is optionally substituted with one or more substituents, each independently selected from the group consisting of:
a halogen atom,
(C1-4)alkyl optionally substituted with one or more fluoro atoms,
(C3-6)cycloalkyl optionally with a substituent selected from the group consisting of (C1-4)alkyl and a halogen atom, and
—(C1-3)alkylene-(C3-6)cycloalkyl optionally substituted with a substituent selected from the group consisting of (C1-4)alkyl and a halogen atom, and
(C1-4)alkoxy optionally substituted with one or more fluoro atoms,
(C3-6)cycloalkoxy optionally substituted with a substituent selected from the group consisting of (C1-4)alkyl and a halogen atom.
—S—(C1-4)alkyl,
—SF5,
*—(CH₂)_{q}—** wherein * and ** are attached to two adjacent C atoms in the 5- to 6-membered ring and q is an integer of 3 to 4
*—CH₂—O—CH₂—CH₂—**, *—O—CH₂—CH₂—CH₂—**, *—CH₂—O—CH₂—O—** and *—O—CH₂—CH₂—O—**, wherein * and ** are attached to two adjacent C atoms in the 5- to 6-membered ring and
*—O—CH₂—O—** or *—O—CH₂—CH₂—**, wherein * and ** are attached to two adjacent C atoms in the 5- to 6-membered ring.

2. A compound according to claim 1, wherein R1 is selected from the group consisting of —CH₂—COOH, —(CH₂)₂—COOH, —(CH₂)₃—COOH, —(CH₂)₄—COOH, —(CH₂)₅—COOH, —CHCH₃—COOH, —CH₂—CHCH₃—COOH, —CHCH₃—CH₂—COOH, —CH₂—C(CH₃)₂—COOH, —C(CH₃)₂—CH₂—COOH, —CH₂—CHCH₃—CH₂—COOH, —CH₂—C(CH₃)₂—CH₂—COOH, —(CH₂)₂—CHCH₃—COOH, —(CH₂)₂—C(CH₃)₂—COOH, —C(CH₃)₂—(CH₂)₂—COOH, —CHCH₃—(CH₂)₂—COOH, —CH₂—COO(C1-4)alkyl, —(CH₂)₂—COO(C1-4)alkyl, —(CH₂)₃—COO(C1-4)alkyl, —CH₂—CHCH₃—CH₂—COO(C1-4)alkyl, —CH₂—C(CH₃)₂—CH₂—COO(C1-4)alkyl, —(CH₂)₂—CHCH₃—COO(C1-4)alkyl, —C(CH₃)₂—(CH₂)₂—COO(C1-4)alkyl, 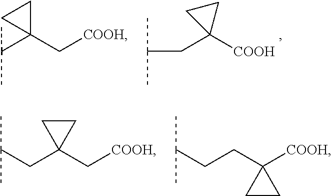
3-carboxycyclobutyl, 3-(carboxymethyl)cyclobutyl, (3-carboxycyclobutyl)methyl, 3-carboxy-3-methylcyclobutyl, 3-carboxycyclobutyl (C1-4)alkyl ester, 3-(carboxymethyl)cyclobutyl (C1-4)alkyl ester, (3-carboxycyclobutyl)methyl, 3-carboxy-3-methylcyclobutyl (C1-4)alkyl ester, —CH₂—CF₂—CH₂—COOH, —CH₂—CHF—CH₂—COOH, and —CH₂—CF(CH₃)—CH₂—COOH, wherein for each of these R1 groups one or more carbon atoms, each independently, is optionally substituted with one or more F or Cl atoms.

3. A compound according to claim 2, wherein R1 is selected from the group consisting of —CH₂—COOH, —CH₂—CH₂—COOH, —CH₂—CH₂—CH₂—COOH, —CH₂—CH(CH₃)—CH₂—COOH, —CH₂—CHF—CH₂—COOH, —CH₂—CF₂—CH₂—COOH, —CH₂—CF(CH₃)—CH₂—COOH and -1,3-cyclobutylene-COOH.

4. A compound according to claim 1 wherein R2 is hydrogen or R2 is selected from the group consisting of fluoro, methyl optionally substituted with one or more halogen atoms, ethyl optionally substituted with one or more halogen atoms and isopropanyl optionally substituted with one or more halogen atoms, wherein R2 is preferably absent.

5. A compound according to claim 1 wherein L1 is selected from the group consisting of —C≡C—, —O—CH₂— and —O—CH₂—, wherein L1 is preferably —C≡C— or —O—CH₂— with C being attached to R3.

6. A compound according to claim 1, wherein the 5- to 6-membered ring of R3 is selected from the group consisting of:
phenyl, optionally substituted with phenyl;
an unsaturated 5-membered ring with a single heteroatom selected from the group consisting of thiophenyl, furan and pyrrolyl;
an unsaturated 5-membered ring with two heteroatoms selected from the group consisting of pyrazolyl, imidazolyl, oxazole, thiazolyl and isothiazolyl;
an unsaturated 6-membered ring with a single heteroatom selected from the group consisting of pyridinyl;
an unsaturated 6-membered ring with two heteroatoms selected from pyrimidinyl, pyrazinyl and pyridazinyl; and
a saturated 5-6 membered ring selected from the group consisting of cyclopentyl, cyclohexyl, tetrahydropyranyl and tetrayhydrofuranyl.

7. A compound according to claim 6, wherein the 5- to 6-membered ring of R3 is an unsaturated ring selected from the group consisting of phenyl, pyridinyl, thiophenyl, pyrazolyl, thiazolyl, pyrimidinyl, pyrazinyl and pyridazinyl.

8. A compound according to claim 6, wherein the 5- to 6-membered ring of R3 is a saturated 5-6 membered ring selected from the group consisting of cyclopentyl, cyclohexyl, tetrahydropyranyl and tetrayhydrofuranyl.

9. A compound according to claim 1, wherein the 5- to 6-membered ring of R3 is optionally substituted with one or more substituents, each independently selected from the group consisting of:
—F, —Cl,
—CH₃, —CH₂—CH₃, —CHCH₃—CH₃, —C(CH₃)₂—CH₃, —(CH₂)₂—CH₃, —CH₂—CHCH₃—CH₃, —CHCH₃—CH₂—CH₃, —CH₂—C(CH₃)₂—CH₃, —C(CH₃)₂—CH₂—CH₃,
—CF₃, —O—CF₃
-cyclopropyl, -cyclybutyl, -cyclopentyl,
—(CH₂)ₚ-cyclopropyl, —(CH₂)ₚ-cyclobutyl and —(CH₂)ₚ-cyclopentyl, wherein p is an integer from 0 to 3,
—O—CH₃, —O—CH₂—CH₃, —O—CHCH₃—CH₃, —O—(CH₂)₂—CH₃, —O—CH₂—CHCH₃—CH₃, —O—CHCH₃—CH₂—CH₃, —O—CH₂—C(CH₃)₂—CH₃, —O—C(CH₃)₂—CH₂—CH₃, —O—C(CH₃)₃,
—O-cyclopentyl, O-cyclobutyl, O-cyclopropyl,
—S—CH₃, —S—CH₂—CH₃, —S—CHCH₃—CH₃
*—(CH₂)_{q}—** wherein * and ** are attached to two adjacent C atoms in the 5- to 6-membered ring and p is an integer of 3 to 4,
*—CH₂—O—CH₂—CH₂—**, *—O—CH₂—CH₂—CH₂—**, *—CH₂—O—CH₂—O—** or *—O—CH₂—CH₂—O—**wherein * and ** are attached to two adjacent C atoms in the 5- to 6-membered ring and
*—O—CH₂—O—** or *—O—CH₂—CH₂—**, wherein * and ** are attached to two adjacent C atoms in the 5- to 6-membered ring,
wherein for each of these groups, one or more carbon atoms may optionally be substituted, each independently, with a substituent selected from F and Cl.

10. A compound according to claim 9, wherein the 5- to 6-membered ring of R3 is optionally substituted with one or more substituents, each independently selected from the group consisting of:
—F, —Cl,
—CH₃, —CH₂—CH₃, —CHCH₃—CH₃, —C(CH₃)₂—CH₃, —(CH₂)₂—CH₃, —CH₂—CHCH₃—CH₃, —CHCH₃—CH₂—CH₃, —CH₂—C(CH₃)₂—CH₃, —C(CH₃)₂—CH₂—CH₃,
—CF₃, —O—CF₃
-cyclopropyl, -cyclybutyl, -cyclopentyl,
—(CH₂)ₚ-cyclopropyl, —(CH₂)ₚ-cyclobutyl and —(CH₂)ₚ-cyclopentyl, wherein p is an integer from 0 to 3,
—O—CH₃, —O—CH₂—CH₃, —O—CHCH₃—CH₃, —O—(CH₂)₂—CH₃, —O—CH₂—CHCH₃—CH₃, —O—CHCH₃—CH₂—CH₃, —O—CH₂—C(CH₃)₂—CH₃, —O—C(CH₃)₂—CH₂—CH₃, —O—C(CH₃)₃,
—O-cyclopentyl, O-cyclobutyl, O-cyclopropyl, and
—S—CH₃, —S—CH₂—CH₃, —S—CHCH₃—CH₃.

11. A compound according to claim 1 wherein the 5- to 6-membered ring of R3 is selected from the group consisting of phenyl, pyridinyl and thiophenyl, and wherein L1 is selected from the group consisting of —C≡C— and —O—CH₂—.

12. A compound according to claim 1, wherein R3 is selected from the group consisting of cyclopentyl, cyclohexyl, tetrahydropyranyl and tetrayhydrofuranyl, and wherein L1 is selected from the group consisting of —C≡C— and —O—CH₂—.

13. A compound according to claim 1 or a pharmaceutically acceptable salt thereof selected from the group consisting of
2-(6′-((2-chloro-6-ethylbenzyl)oxy)-1′H-spiro[cyclopropane-1,4′-isoquinolin]-2′(3′H)-yl)acetic acid,
3-(6′-((2,3-difluorophenyl)ethynyl)-1′H-spiro[cyclopropane-1,4′-isoquinolin]-2′(3′H)-yl)propanoic acid,
3-(6′-((2,6-dichlorophenyl)ethynyl)-1′H-spiro[cyclopropane-1,4′-isoquinolin]-2′(3′H)-yl)propanoic acid,
3-(6′-((4-ethoxyphenyl)ethynyl)-1′H-spiro[cyclopropane-1,4′-isoquinolin]-2′(3′H)-yl)propanoic acid,
3-(6′-((4-isopropylphenyl)ethynyl)-1′H-spiro[cyclopropane-1,4′-isoquinolin]-2′(3′H)-yl)propanoic acid,
3-(6′-((5-isopropoxypyridin-2-yl)ethynyl)-1′H-spiro[cyclopropane-1,4′-isoquinolin]-2′(3′H)-yl)cyclobutanecarboxylic acid,
3-(6′-(thiophen-3-ylethynyl)-1′H-spiro[cyclopropane-1,4′-isoquinolin]-2′(3′H)-yl)propanoic acid,
3-methyl-4-(6′-(thiophen-3-ylethynyl)-1′H-spiro[cyclopropane-1,4′-isoquinolin]-2′(3′H)-yl)butanoic acid,
4-(6′-((2,6-dichlorobenzyl)oxy)-1′H-spiro[cyclopropane-1,4′-isoquinolin]-2′(3′H)-yl)butanoic acid,
4-(6′-((2-(trifluoromethoxy)benzyl)oxy)-1′H-spiro[cyclopropane-1,4′-isoquinolin]-2′(3′H)-yl)butanoic acid,
4-(6′-((2-chloro-6-ethylbenzyl)oxy)-1′H-spiro[cyclopropane-1,4′-isoquinolin]-2′(3′H)-yl)butanoic acid,
4-(6′-((2-methylbenzyl)oxy)-1′H-spiro[cyclopropane-1,4′-isoquinolin]-2′(3′H)-yl)butanoic acid,
4-(6′-((3-fluoro-4-(trifluoromethoxy)phenyl)ethynyl)-1′H-spiro[cyclopropane-1,4′-isoquinolin]-2′(3′H)-yl)-3-methylbutanoic acid,
4-(6′-((3-fluoro-4-(trifluoromethoxy)phenyl)ethynyl)-1′H-spiro[cyclopropane-1,4′-isoquinolin]-2′(3′H)-yl)butanoic acid,
4-(6′-((3-fluorobenzyl)oxy)-1′H-spiro[cyclopropane-1,4′-isoquinolin]-2′(3′H)-yl)butanoic acid,
4-(6′-((4-(cyclopentyloxy)phenyl)ethynyl)-1′H-spiro[cyclopropane-1,4′-isoquinolin]-2′(3′H)-yl)butanoic acid,
4-(6′-((4-(trifluoromethoxy)phenyl)ethynyl)-1′H-spiro[cyclopropane-1,4′-isoquinolin]-2′(3′H)-yl)butanoic acid,
4-(6′-((4-ethoxy-3-fluorophenyl)ethynyl)-1′H-spiro[cyclopropane-1,4′-isoquinolin]-2′(3′H)-yl)-3-methylbutanoic acid,
4-(6′-((4-isopropoxyphenyl)ethynyl)-1′H-spiro[cyclopropane-1,4′-isoquinolin]-2′(3′H)-yl)-3-methylbutanoic acid,
4-(6′-((4-isopropoxyphenyl)ethynyl)-1′H-spiro[cyclopropane-1,4′-isoquinolin]-2′(3′H)-yl)butanoic acid,
4-(6′-((4-isopropylphenyl)ethynyl)-1′H-spiro[cyclopropane-1,4′-isoquinolin]-2′(3′H)-yl)-3-methylbutanoic acid,
4-(6′-((6-ethoxy-4-methylpyridin-3-yl)ethynyl)-1′H-spiro[cyclopropane-1,4′-isoquinolin]-2′(3′H)-yl)-3-methylbutanoic acid,
4-(6′-((6-isopropoxypyridin-3-yl)ethynyl)-1′H-spiro[cyclopropane-1,4′-isoquinolin]-2′(3′H)-yl)-3-methylbutanoic acid and
4-(6′-((6-isopropoxypyridin-3-yl)ethynyl)-1′H-spiro[cyclopropane-1,4′-isoquinolin]-2′(3′H)-yl)butanoic acid.

14-22. (canceled)
